(19) United States
(12) Patent Application Publication
(10) Pub. No.: US 2016/0101414 A1
(43) Pub. Date: Apr. 14, 2016

(54) HIGHLY Z-SELECTIVE AND ENANTIOSELECTIVE RING OPENING/CROSS METATHESIS CATALYZED BY A RESOLVED STEREOGENIC-AT-RU COMPLEX

(71) Applicant: CALIFORNIA INSTITUTE OF TECHNOLOGY, Pasadena, CA (US)

(72) Inventors: John HARTUNG, Pasadena, CA (US); Robert H. GRUBBS, South Pasadena, CA (US)

(21) Appl. No.: 14/890,674

(22) PCT Filed: May 15, 2014
(86) PCT No.: PCT/US2014/038280

§ 371 Date: Nov. 12, 2015

Related U.S. Application Data
(60) Provisional application No. 61/933,586, filed on Jan. 30, 2014.
(60) Provisional application No. 61/838,673, filed on Jun. 24, 2013.
(60) Provisional application No. 61/823,539, filed on May 15, 2013.

(51) Int. Cl. B01J 31/22; C07D 307/93; C07C 67/475; C07C 69/007; C07C 41/18; C07C 43/295; C07C 269/06; C07C 271/16; C07F 5/04; C07C 43/285; C07D 209/52; C07C 201/02; C07C 203/10; C07F 7/18; C07H 3/02; C07F 15/00

(52) U.S. Cl. CPC B01J 31/2278; C07F 15/0046; C07D 307/93; C07C 67/475; C07C 69/007; C07C 41/18; C07C 43/295; C07C 269/06; C07C 271/16; C07F 5/04; C07C 43/285; C07D 209/52; C07C 201/02; C07C 203/10; C07F 7/1888; C07F 7/1848; C07H 3/02; B01J 2231/543; B01J 2531/821; C07C 2101/08; C07C 2102/08

(57) ABSTRACT

This invention relates generally to enantiomerically enriched C—H activated ruthenium olefin metathesis catalyst compounds which are stereogenic at ruthenium, to the preparation of such compounds, and the use of such catalysts in the metathesis of olefins and olefin compounds, more particularly, in the use of such catalysts in enantio- and Z-selective olefin metathesis reactions. The invention has utility in the fields of catalysis, organic synthesis, polymer chemistry, and industrial and fine chemicals chemistry.

### RELATED APPLICATIONS

This application claims the benefit of U.S. Provisional Patent Application No. 61/823,539, filed May 15, 2013, U.S. Provisional Patent Application No. 61/838,673, filed Jun. 24, 2013, and U.S. Provisional Patent Application No. 61/933,586, filed Jan. 30, 2014, the contents of each are incorporated herein by reference.

### STATEMENT REGARDING FEDERALLY SPONSORED RESEARCH AND DEVELOPMENT

This invention was made with government support under Grant No. 5R01GM031332-27 awarded by the National Institutes of Health and Grant No. CHE-1048404 awarded by the National Science Foundation. The U.S. Government has certain rights in this invention.

### TECHNICAL FIELD

This invention relates generally to enantiomerically enriched C—H activated ruthenium olefin metathesis catalyst compounds which are stereogenic at ruthenium, to the preparation of such compounds, and the use of such catalysts in the metathesis of olefins and olefin compounds, more particularly, in the use of such catalysts in enantio- and Z-selective olefin metathesis reactions. The invention has utility in the fields of catalysis, organic synthesis, polymer chemistry, and industrial and fine chemicals chemistry.

### BACKGROUND

Since its discovery in the 1950s, olefin metathesis has emerged as a valuable synthetic method for the formation of carbon-carbon double bonds. In particular, its recent advances in applications to organic syntheses and polymer syntheses mostly rely on developments of well-defined catalysts (see (a) Cossy, J.; Arseniyadis, S.; Meyer, C., Metathesis in Natural Product Synthesis: Strategies, Substrates, and Catalysts. 1st ed.; Wiley-VCH: Weinheim, Germany, 2010; (b) Nicolaou, K. C.; Bulger, P. G.; Sarlah, D., Angew. Chem., Int. Ed. 2005, 44, 4490-4527; (c) Mutlu, H.; de Espinosa, L. M.; Meier, M. A. R., Chem. Soc. Rev. 2011, 40, 1404-1445; (d) Leitgeb, A.; Wappel, J.; Slugovc, C., Polymer 2010, 51, 2927-2946; (e) Buchmeiser, M. R., Macromol. Symp. 2010, 298, 17-24; (f) Sutthasupa, S.; Shiotsuki, M.; Sanda, F., Polymer J. 2010, 42, 905-915; (g) Binder, J. B.; Raines, R. T., Curr. Opin. Chem. Biol. 2008, 12, 767-773). Among attempts to improve catalyst efficiency over the past decade, one of the most attractive frontiers has been selective synthesis of stereo-controlled olefin product. However, most catalysts give higher proportion of thermodynamically favored E isomer of olefin in products. This fundamental nature of olefin metathesis limits its applications to some reactions including natural product synthesis. Furthermore, asymmetric olefin metathesis methodologies are desirable for the synthesis of enantiopure natural products and other biologically-relevant molecules (see Hoveyda, A. H.; Malcolmson, S. J.; Meek, S. J.; Zhugralin, A. R., Angew. Chem., Int. Ed. 2010, 49, 34-44). Thus, an enantioselective catalyst which also gives high Z-isomer of olefin product is expected to open a new convenient route to value-added products. Consequently, the development of chiral catalysts for methods such as asymmetric ring opening/cross metathesis (AROCM) is a field of ongoing interest (see Kress, S.; Blechert, S., Chem. Soc. Rev. 2012, 41, 4389-4408).

The earliest examples of such catalysts contained Molybdenum, and while capable of generating AROCM products in high ee (80-90%), suffered from limited substrate scope and functional group compatibility (see (a) Fujimura, O.; Grubbs, R. H., J. Am. Chem. Soc. 1996, 118, 2499-2500; (b) Fujimura, O.; Grubbs, R. H., J. Org. Chem. 1998, 63, 824-832; (c) La, D. S.; Ford, J. G.; Sattely, E. S.; Bonitatebus, P. J.; Schrock, R. R.; Hoveyda, A. H., J. Am. Chem. Soc. 1999, 121, 11603-11604; (d) La, D. S.; Sattely, E. S.; Ford, J. G.; Schrock, R. R.; Hoveyda, A. H., J. Am. Chem. Soc. 2001, 123, 7767-7778; (e) Tsang, W. C. P.; Jernelius, J. A.; Cortez, G. A.; Weatherhead, G. S.; Schrock, R. R.; Hoveyda, A. H., J. Am. Chem. Soc. 2003, 125, 2591-2596). Ruthenium-based catalysts have been developed wherein the chirality is built into the N-heterocyclic carbene (NHC) ligand (see (a) Seiders, T. J.; Ward, D. W.; Grubbs, R. H., Org. Lett. 2001, 3, 3225-3228; (b) Berlin, J. M.; Goldberg, S. D.; Grubbs, R. H., Angew. Chem., Int. Ed. 2006, 45, 7591-7595; (c) Funk, T. W.; Berlin, J. M.; Grubbs, R. H., J. Am. Chem. Soc. 2006, 128, 1840-1846; (d) Savoie, J.; Stenne, B.; Collins, S. K., Adv. Synth. Catal. 2009, 351, 1826-1832; (e) Stenne, B.; Timperio, J.; Savoie, J.; Dudding, T.; Collins, S. K., Org. Lett. 2010, 12, 2032-2035; (f) Tiede, S.; Berger, A.; Schlesiger, D.; Rost, D.; Luehl, A.; Blechert, S., Angew. Chem., Int. Ed. 2010, 49, 3972-3975; (g) Kannenberg, A.; Rost, D.; Eibauer, S.; Tiede, S.; Blechert, S., Angew. Chem., Int. Ed. 2011, 50, 3299-3302; (h) Van Veldhuizen, J. J.; Garber, S. B.; Kingsbury, J. S.; Hoveyda, A. H., J. Am. Chem. Soc. 2002, 124, 4954-4955; (i) Van Veldhuizen, J. J.; Gillingham, D. G.; Garber, S. B.; Kataoka, O.; Hoveyda, A. H., J. Am. Chem. Soc. 2003, 125, 12502-12508; (j) Van Veldhuizen, J. J.; Campbell, J. E.; Giudici, R. E.; Hoveyda, A. H., J. Am. Chem. Soc. 2005, 127, 6877-6882). Most of these molybdenum and ruthenium catalyst are capable of performing AROCM with high levels of E-selectivity (up to >98% E) (For an early example of Z-selective ROCM see (a) Randall, M. L.; Tallarico, J. A.; Snapper, M. L., J. Am. Chem. Soc. 1995, 117, 9610-9611; (b) Tallarico, J. A.; Randall, M. L.; Snapper, M. L., Tetrahedron 1997, 53, 16511-16520). More recently, Z-selective AROCM of oxabicycles has been achieved with molybdenum catalysts (see (a) Ibrahem, I.; Yu, M.; Schrock, R. R.; Hoveyda, A. H., J. Am. Chem. Soc. 2009, 131, 3844-3845; (b) Yu, M.; Ibrahem, I.; Hasegawa, M.; Schrock, R. R.; Hoveyda, A. H., J. Am. Chem. Soc. 2012, 134, 2788-2799). While Z-selective AROCM has been accomplished with ruthenium catalysts, thus far it has been limited to reactions involving heteroatom-substituted α-olefin cross partners (see (a) Khan, R. K. M.; O'Brien, R. V.; Torker, S.; Li, B.; Hoveyda, A. H., J. Am. Chem. Soc. 2012, 134, 12774-12779; (b) Khan, R. K. M.; Zhugralin, A. R.; Torker, S.; O'Brien, R. V.; Lombardi, P. J.; Hoveyda, A. H., J. Am. Chem. Soc. 2012, 134, 12438-12441).

The formation of multiple stereocenters in a single catalytic transformation is a powerful approach to the synthesis of stereochemically complex targets. While the development of such a transformation must overcome the challenge of simultaneously controlling diastereo- and enantioselectivity, the end result can reduce the step count of a synthesis and improve its atom economy. One commonly encountered motif is the vicinal diol, which is pervasive throughout natural products and ligands for asymmetric transformations. While the problem of introducing vicinal diols in high enantiopurity has largely been solved by the Sharpless asymmetric dihydroxylation (AD), the formation of 1,2-anti diols remains challenging due to the low enantioselectivity observed in the AD of cis-1,2 disubstituted alkenes (see H. C. Kolb, M. S. Vannieuwenhze, K. B. Sharpless, Chem. Rev. 1994, 94, 2483-2547). Accordingly, a number of methods have been developed for the enantioselective formation of 1,2-anti diols, including asymmetric epoxidation/hydrolysis (see (a) S. M. Lim, N. Hill, A. G. Myers, J. Am. Chem. Soc. 2009, 131, 5763-5765; (b) L. Albrecht, H. Jiang, G. Dickmeiss, B. Gschwend, S. G. Hansen, K. A. Jorgensen, J. Am. Chem. Soc. 2010, 132, 9188-9196), glycolate aldol (see (a) T. Mukaiyama, N. Iwasawa, Chem. Lett. 1984, 753-756; (b) D. A. Evans, J. R. Gage, J. L. Leighton, A. S. Kim, J. Org. Chem. 1992, 57, 1961-1963; (c) W. Notz, B. List, J. Am. Chem. Soc. 2000, 122, 7386-7387; (d) M. T. Crimmins, P. J. McDougall, Org. Lett. 2003, 5, 591-594; (e) A. B. Northrup, D. W. C. MacMillan, Science 2004, 305, 1752-1755; (f) A. B. Northrup, I. K. Mangion, F. Hettche, D. W. C. MacMillan, Angew. Chem. 2004, 116, 2204-2206; Angew. Chem., Int. Ed. 2004, 43, 2152-2154; (g) S. E. Denmark, W.-J. Chung, Angew. Chem. 2008, 120, 1916-1918; Angew. Chem., Int. Ed. 2008, 47, 1890-1892), iterative cross metathesis/allylic substitution (see (a) J. K. Park, D. T. McQuade, Angew. Chem. 2012, 124, 2771-2775; Angew. Chem., Int. Ed. 2012, 51, 2717-2721; (b) D. Kim, J. S. Lee, S. B. Kong, H. Han, Angew. Chem. 2013, 125, 4297-4300; Angew. Chem., Int. Ed. 2013, 52, 4203-4206), nucleophilic addition to aldehydes (see (a) E. El-Sayed, N. K. Anand, E. M. Carreira, Org. Lett. 2001, 3, 3017-3020; (b) T. Luanphaisarnnont, C. O. Ndubaku, T. F. Jamison, Org. Lett. 2005, 7, 2937-2940; (c) S. B. Han, H. Han, M. J. Krische, J. Am. Chem. Soc. 2010, 132, 1760-1761), desymmetrizing monofunctionalization (see Y. Zhao, J. Rodrigo, A. H. Hoveyda, M. L. Snapper, Nature 2006, 443, 67-70), and allene hydroboration/aldehyde allylation (see H. C. Brown, G. Narla, J. Org. Chem. 1995, 60, 4686-4687). In contrast to many of these methods, an asymmetric ring opening/cross metathesis (AROCM) approach (Scheme 4) would consolidate the transformation into a single step and generate a differentiated 1,5-diene fragment in a convergent manner.

Asymmetric olefin metathesis is a powerful C—C bond forming reaction and has enabled the synthesis of stereochemically complex bioactive compounds (see A. H. Hoveyda, S. J. Malcolmson, S. J. Meek, A. R. Zhugralin, Angew. Chem. 2010, 122, 38-49; Angew. Chem., Int. Ed. 2010, 49, 34-44). Advances in stereoselective olefin metathesis have resulted in the development of catalysts capable of forming products with high diastereo- and enantioselectivity (For a recent review, see A. Fürstner, Science 2013, 341, 1229713. For leading references, see (a) K. Endo, R. H. Grubbs, J. Am. Chem. Soc. 2011, 133, 8525-8527; (b) B. K. Keitz, K. Endo, P. R. Patel, M. B. Herbert, R. H. Grubbs, J. Am. Chem. Soc. 2012, 134, 693-699; (c) L. E. Rosebrugh, M. B. Herbert, V. M. Marx, B. K. Keitz, R. H. Grubbs, J. Am. Chem. Soc. 2013, 135, 1276-1279; (d) M. M. Flook, A. J. Jiang, R. R. Schrock, P. Mueller, A. H. Hoveyda, J. Am. Chem. Soc. 2009, 131, 7962-7963; (e) S. J. Meek, R. V. O'Brien, J. Llaveria, R. R. Schrock, A. H. Hoveyda, Nature 2011, 471, 461-466; (f) R. K. M. Khan, S. Torker, A. H. Hoveyda, J. Am. Chem. Soc. 2013, 135, 10258; For a recent review, see (a) S. Kress, S. Blechert, Chem. Soc. Rev. 2012, 41, 4389-4408; for leading references, see (b) J. M. Berlin, S. D. Goldberg, R. H. Grubbs, Angew. Chem. 2006, 118, 7753-7757; Angew. Chem., Int. Ed. 2006, 45, 7591-7595; (c) T. W. Funk, J. M. Berlin, R. H. Grubbs, J. Am. Chem. Soc. 2006, 128, 1840-1846; (d) J. Savoie, B. Stenne, S. K. Collins, Adv. Synth. Catal. 2009, 351, 1826-1832; (e) B. Stenne, J. Timperio, J. Savoie, T. Dudding, S. K. Collins, Org. Lett. 2010, 12, 2032-2035; (f) S. Tiede, A. Berger, D. Schlesiger, D. Rost, A. Luhl, S. Blechert, Angew. Chem. 2010, 122, 4064-4067; Angew. Chem., Int. Ed. 2010, 49, 3972-3975; (g) A. Kannenberg, D. Rost, S. Eibauer, S. Tiede, S. Blechert, Angew. Chem. 2011, 123, 3357-3360; Angew. Chem., Int. Ed. 2011, 50, 3299-3302; (h) R. K. M. Khan, R. V. O'Brien, S. Torker, B. Li, A. H. Hoveyda, J. Am. Chem. Soc. 2012, 134, 12774-12779; (i) M. Yu, I. Ibrahem, M. Hasegawa, R. R. Schrock, A. H. Hoveyda, J. Am. Chem. Soc. 2012, 134, 2788-2799). Although the ROCM of cyclobutenes to form racemic products has been demonstrated (see (a) M. L. Randall, J. A. Tallarico, M. L. Snapper, J. Am. Chem. Soc. 1995, 117, 9610-9611; (b) M. L. Snapper, J. A. Tallarico, M. L. Randall, J. Am. Chem. Soc. 1997, 119, 1478-1479; (c) J. A. Tallarico, M. L. Randall, M. L. Snapper, Tetrahedron 1997, 53, 16511-16520; (d) T. O. Schrader, M. L. Snapper, J. Am. Chem. Soc. 2002, 124, 10998-11000; (e) B. H. White, M. L. Snapper, J. Am. Chem. Soc. 2003, 125, 14901-14904), previous studies of their AROCM reactions have afforded products with low enantioenrichment (see M. Yu, I. Ibrahem, M. Hasegawa, R. R. Schrock, A. H. Hoveyda, J. Am. Chem. Soc. 2012, 134, 2788-2799).

Despite the advances achieved in the art, a continuing need therefore exists for further improvements in the areas of Z-selective AROCM (see (a) Endo, K.; Grubbs, R. H., J. Am. Chem. Soc. 2011, 133, 8525-8527; (b) Keitz, B. K.; Endo, K.; Herbert, M. B.; Grubbs, R. H., J. Am. Chem. Soc. 2011, 133, 9686-9688; (c) Keitz, B. K.; Endo, K.; Patel, P. R.; Herbert, M. B.; Grubbs, R. H., J. Am. Chem. Soc. 2012, 134, 693-699; (d) Rosebrugh, L. E.; Herbert, M. B.; Marx, V. M.; Keitz, B. K.; Grubbs, R. H., J. Am. Chem. Soc. 2013, 135, 1276-1279). The present invention is directed to addressing one or more of those concerns.

### SUMMARY

The invention is directed to addressing one or more of the aforementioned concerns, and, in one embodiment, provides an enantioenriched C—H activated catalyst compound composed of a Group 8 transition metal complex and a chelating ligand structure formed from the metal center M, a neutral electron donor ligand L¹, and a 2-electron anionic donor bridging moiety, Q*. A general structure of catalyst compounds according to the invention is shown below.

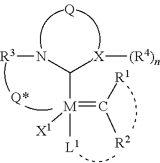

wherein, M is a Group 8 transition metal (e.g., Ru or Os); X¹ is an anionic ligand; L¹ is a neutral two electron ligand, where L¹ may connect with R²; R¹ and R² are independently selected from hydrogen, hydrocarbyl, substituted hydrocarbyl, heteroatom-containing hydrocarbyl, substituted heteroatom-containing hydrocarbyl, and functional groups, and wherein R¹ may connect with R² and/or L¹; Q*is a 2-electron anionic donor bridging moiety (e.g., alkyl, aryl, carboxylate, alkoxy, aryloxy, or sulfonate, etc.).

We have discovered that enantiopure versions of these catalysts exhibit both high Z-selectivity and enantioselectivity in AROCM due to the rigidity imparted by the heterocyclic carbene-metal chelate.

In summary, we have developed an enantioenriched ruthenium metathesis catalyst capable of highly Z-selective and enantioselective ROCM. An NHC ligand that chelates through a Ru—C bond is key to the design of the catalyst, which features a stereogenic Ru atom. The reaction is amenable to modification of both the α-olefin and norbornene component, which significantly broadens the scope of this methodology.

Furthermore, the highly enantioselective synthesis of 1,2-anti diols was accomplished by the application of catalyst 4 to the AROCM of cis-dioxygenated cyclobutenes. The reaction is robust, tolerating modifications in reaction conditions and substitution on the reactants. Enantioenrichment of the major Z isomers was exceptionally high, ranging from 89-99% ee. The rapid synthesis of insect pheromone (+)-endo brevicomin was accomplished, affording the natural product in 95% ee. A 1,5-diene generated by the AROCM reaction was chemoselectively functionalized to afford ribose derivative 21, demonstrating the utility of the building blocks afforded by the title reaction.

### BRIEF DESCRIPTION OF THE DRAWINGS

FIG. 1 is a graph demonstrating the E/Z ratio versus conversion for the reaction leading to product 9e as described in the Examples.

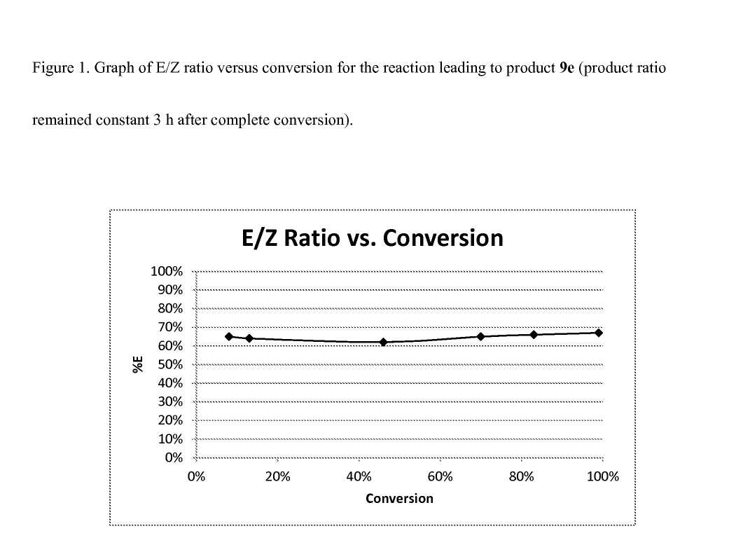

FIG. 2 depicts the X-ray crystal structure (ORTEP drawing) of complex 3 as described in the Examples.

FIG. 3. X-ray crystal structure (ORTEP drawing) of compound ent-S3 as described in the Examples.

### DETAILED DESCRIPTION

### Terminology and Definitions

Unless otherwise indicated, the invention is not limited to specific reactants, substituents, catalysts, reaction conditions, or the like, as such may vary. It is also to be understood that the terminology used herein is for the purpose of describing particular embodiments only and is not to be interpreted as being limiting.

As used in the specification and the appended claims, the singular forms “a,” “an,” and “the” include plural referents unless the context clearly dictates otherwise. Thus, for example, reference to “an α-olefin” includes a single α-olefin as well as a combination or mixture of two or more α-olefins, reference to “a substituent” encompasses a single substituent as well as two or more substituents, and the like.

As used in the specification and the appended claims, the terms “for example,” “for instance,” “such as,” or “including” are meant to introduce examples that further clarify more general subject matter. Unless otherwise specified, these examples are provided only as an aid for understanding the invention, and are not meant to be limiting in any fashion.

In this specification and in the claims that follow, reference will be made to a number of terms, which shall be defined to have the following meanings:

The term “alkyl” as used herein refers to a linear, branched, or cyclic saturated hydrocarbon group typically although not necessarily containing 1 to about 24 carbon atoms, preferably 1 to about 12 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of 1 to 6 carbon atoms, and the specific term “cycloalkyl” intends a cyclic alkyl group, typically having 4 to 8, preferably 5 to 7, carbon atoms. The term “substituted alkyl” refers to alkyl substituted with one or more substituent groups, and the terms “heteroatom-containing alkyl” and “heteroalkyl” refer to alkyl in which at least one carbon atom is replaced with a heteroatom. If not otherwise indicated, the terms “alkyl” and “lower alkyl” include linear, branched, cyclic, unsubstituted, substituted, and/or heteroatom-containing alkyl and lower alkyl, respectively.

The term “alkylene” as used herein refers to a difunctional linear, branched, or cyclic alkyl group, where “alkyl” is as defined above.

The term “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Preferred alkenyl groups herein contain 2 to about 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkenylene” as used herein refers to a difunctional linear, branched, or cyclic alkenyl group, where “alkenyl” is as defined above.

The term “alkynyl” as used herein refers to a linear or branched hydrocarbon group of 2 to about 24 carbon atoms containing at least one triple bond, such as ethynyl, n-propynyl, and the like. Preferred alkynyl groups herein contain 2 to about 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms. Analogously, “alkenyloxy” and “lower alkenyloxy” respectively refer to an alkenyl and lower alkenyl group bound through a single, terminal ether linkage, and “alkynyloxy” and “lower alkynyloxy” respectively refer to an alkynyl and lower alkynyl group bound through a single, terminal ether linkage.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituents in which at least one carbon atom is replaced with a heteroatom, as will be described in further detail infra.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 24 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Preferred alkaryl and aralkyl groups contain 6 to 24 carbon atoms, and particularly preferred alkaryl and aralkyl groups contain 6 to 16 carbon atoms. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. The terms “alkaryloxy” and “aralkyloxy” refer to substituents of the formula —OR wherein R is alkaryl or aralkyl, respectively, as just defined.

The term “acyl” refers to substituents having the formula —(CO)-alkyl, —(CO)-aryl, or —(CO)-aralkyl, and the term “acyloxy” refers to substituents having the formula —O(CO)-alkyl, —O(CO)-aryl, or —O(CO)-aralkyl, wherein “alkyl,” “aryl, and “aralkyl” are as defined above.

The terms “cyclic” and “ring” refer to alicyclic or aromatic groups that may or may not be substituted and/or heteroatom containing, and that may be monocyclic, bicyclic, or polycyclic. The term “alicyclic” is used in the conventional sense to refer to an aliphatic cyclic moiety, as opposed to an aromatic cyclic moiety, and may be monocyclic, bicyclic, or polycyclic.

The terms “halo” and “halogen” are used in the conventional sense to refer to a chloro, bromo, fluoro, or iodo substituent.

“Hydrocarbyl” refers to univalent hydrocarbyl radicals containing 1 to about 30 carbon atoms, preferably 1 to about 24 carbon atoms, most preferably 1 to about 12 carbon atoms, including linear, branched, cyclic, saturated, and unsaturated species, such as alkyl groups, alkenyl groups, aryl groups, and the like. The term “lower hydrocarbyl” intends a hydrocarbyl group of 1 to 6 carbon atoms, preferably 1 to 4 carbon atoms, and the term “hydrocarbylene” intends a divalent hydrocarbyl moiety containing 1 to about 30 carbon atoms, preferably 1 to about 24 carbon atoms, most preferably 1 to about 12 carbon atoms, including linear, branched, cyclic, saturated and unsaturated species. The term “lower hydrocarbylene” intends a hydrocarbylene group of 1 to 6 carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and heterohydrocarbylene” refer to hydrocarbylene in which at least one carbon atom is replaced with a heteroatom. Unless otherwise indicated, the term “hydrocarbyl” and “hydrocarbylene” are to be interpreted as including substituted and/or heteroatom-containing hydrocarbyl and hydrocarbylene moieties, respectively.

The term “heteroatom-containing” as in a “heteroatom-containing hydrocarbyl group” refers to a hydrocarbon molecule or a hydrocarbyl molecular fragment in which one or more carbon atoms is replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. It should be noted that a “heterocyclic” group or compound may or may not be aromatic, and further that “heterocycles” may be monocyclic, bicyclic, or polycyclic as described above with respect to the term “aryl.” Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

By “substituted” as in “substituted hydrocarbyl,” “substituted alkyl,” “substituted aryl,” and the like, as alluded to in some of the aforementioned definitions, is meant that in the hydrocarbyl, alkyl, aryl, or other moiety, at least one hydrogen atom bound to a carbon (or other) atom is replaced with one or more non-hydrogen substituents. Examples of such substituents include, without limitation: functional groups referred to herein as “Fn,” such as halo, hydroxyl, sulfhydryl, C₁-C₂₄ alkoxy, C₂-C₂₄ alkenyloxy, C₂-C₂₄ alkynyloxy, C₅-C₂₄ aryloxy, C₆-C₂₄ aralkyloxy, C₆-C₂₄ alkaryloxy, acyl (including C₂-C₂₄ alkylcarbonyl (—CO-alkyl) and C₆-C₂₄ arylcarbonyl (—CO-aryl)), acyloxy (—O-acyl, including C₂-C₂₄ alkylcarbonyloxy (—O—CO-alkyl) and C₆-C₂₄ arylcarbonyloxy (—O—CO-aryl)), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO⁻, carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₁-C₂₄ haloalkyl)-substituted carbamoyl (—(CO)—NH(C₁-C₂₄ alkyl)), di-(C₁-C₂₄ haloalkyl)-substituted carbamoyl (—(CO)—N(C₁-C₂₄ alkyl)₂), mono-(C₅-C₂₄ aryl)-substituted carbamoyl (—(CO)—NH-aryl), di-(C₅-C₂₄ aryl)-substituted carbamoyl (—(CO)—N(C₅-C₂₄ aryl)₂), di-N—(C₁-C₂₄ alkyl), N—(C₅-C₂₄ aryl)-substituted carbamoyl, thiocarbamoyl (—(CS)—NH₂), mono-(C₁-C₂₄ alkyl)-substituted thiocarbamoyl (—(CO)—NH(C₁-C₂₄ alkyl)), di-(C₁-C₂₄ alkyl)-substituted thiocarbamoyl (—(CO)—N(C₁-C₂₄ alkyl)₂), mono-(C₅-C₂₄ aryl)-substituted thiocarbamoyl (—(CO)—NH-aryl), di-(C₅-C₂₄ aryl)-substituted thiocarbamoyl (—(CO)—N(C₅-C₂₄ aryl)₂), di-N—(C₁-C₂₄ alkyl), N—(C₅-C₂₄ aryl)-substituted thiocarbamoyl, carbamido (—NH—(CO)—NH₂), cyano(—C═N), cyanato (—O—C═N), thiocyanato (—S—C═N), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), C₂-C₂₀ alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₀ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), C₅-C₂₄ arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₁-C₂₄ monoalkylaminosulfonyl —SO₂—N(H) alkyl), C₁-C₂₄ dialkylaminosulfonyl —SO₂—N(alkyl)₂, C₅-C₂₄ arylsulfonyl (—SO₂-aryl), boryl (—BH₂), borono (—B(OH)₂), boronato (—B(OR)₂ where R is alkyl or other hydrocarbyl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₂ alkyl, more preferably C₁-C₆ alkyl), C₂-C₂₄ alkenyl (preferably C₂-C₁₂ alkenyl, more preferably C₂-C₆ alkenyl), C₂-C₂₄ alkynyl (preferably C₂-C₁₂ alkynyl, more preferably C₂-C₆ alkynyl), C₅-C₂₄ aryl (preferably C₅-C₁₄ aryl), C₆-C₂₄ alkaryl (preferably C₆-C₁₆ alkaryl), and C₆-C₂₄ aralkyl (preferably C₆-C₁₆ aralkyl).

By “functionalized” as in “functionalized hydrocarbyl,” “functionalized alkyl,” “functionalized olefin,” “functionalized cyclic olefin,” and the like, is meant that in the hydrocarbyl, alkyl, olefin, cyclic olefin, or other moiety, at least one hydrogen atom bound to a carbon (or other) atom is replaced with one or more functional groups such as those described hereinabove. The term “functional group” is meant to include any functional species that is suitable for the uses described herein. In particular, as used herein, a functional group would necessarily possess the ability to react with or bond to corresponding functional groups on a substrate surface.

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term enantioenriched C—H activated catalyst refers to mirror images when one chiral center is present and diastereomers with 2 or more chiral centers are present.

### Catalyst Complexes

In general, the catalyst complexes of the invention comprise a Group 8 metal (M), an alkylidene moiety (═CR¹R²), or more generally (═(C)ₘCR¹R²), an anionic ligand (X¹), a neutral ligand (L¹) and a heterocyclic carbene ligand that is linked to the metal via a 2-electron anionic donor bridging moiety (Q*). The olefin metathesis catalyst complex is preferably a Group 8 transition metal complex having the structure of formula (II)

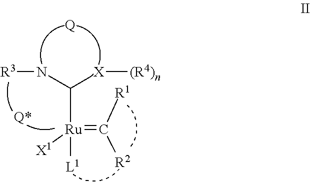

in which:

L¹ is a neutral electron donor ligand;

Q* is a 2-electron anionic donor bridging moiety linking R³ and Ru; and may be hydrocarbylene (including substituted hydrocarbylene, heteroatom-containing hydrocarbylene, and substituted heteroatom-containing hydrocarbylene, such as substituted and/or heteroatom-containing alkylene) or —(CO)—;

Q is a linker, typically a hydrocarbylene linker, including substituted hydrocarbylene, heteroatom-containing hydrocarbylene, and substituted heteroatom-containing hydrocarbylene linkers, wherein two or more substituents on adjacent atoms within Q may also be linked to form an additional cyclic structure, which may be similarly substituted to provide a fused polycyclic structure of two to about five cyclic groups. Q is often, although again not necessarily, a two-atom linkage or a three-atom linkage;

X is an atom selected from C, N, O, S, and P. Since O and S are divalent, n is necessarily zero when X is O or S. Similarly, when X is N or P, then n is 1, and when X is C, then n is 2;

R¹ and R² are independently selected from hydrogen, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and functional groups. R¹ and R² may also be linked to form a cyclic group, which may be aliphatic or aromatic, and may contain substituents and/or heteroatoms. Generally, such a cyclic group will contain 4 to 12, preferably 5, 6, 7, or 8 ring atoms.

R³ and R⁴ are independently selected from hydrogen, hydrocarbyl, substituted hydrocarbyl, heteroatom-containing hydrocarbyl, and substituted heteroatom-containing, hydrocarbyl (e.g., C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), substituted hydrocarbyl (e.g., substituted C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), heteroatom-containing hydrocarbyl (e.g., heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and substituted heteroatom-containing hydrocarbyl (e.g., substituted heteroatom-containing C₁-C₂₀ alkyl, C₂-C₂₀ alkenyl, C₂-C₂₀ alkynyl, C₅-C₂₄ aryl, C₆-C₂₄ alkaryl, C₆-C₂₄ aralkyl, etc.), and functional groups.

X¹ is a bidentate anionic ligand. Typically, X¹ is nitrate, C₁-C₂₀ alkylcarboxylate, C₆-C₂₄ arylcarboxylate, C₂-C₂₄ acyloxy, C₁-C₂₀ alkylsulfonato, C₅-C₂₄ arylsulfonato, C₁-C₂₀ alkylsulfanyl, C₅-C₂₄ arylsulfanyl, C₁-C₂₀ alkylsulfinyl, or C₅-C₂₄ arylsulfinyl. In some embodiments, X¹ is benzoate, pivalate, or nitrate. More specifically, X¹ may be is CF₃CO₂, CH₃CO₂, CH₃CH₂CO₂, CFH₂CO₂, (CH₃)₃CO₂, (CH₃)₂CHCO₂, (CF₃)₂(CH₃)CO₂, (CF₃)(CH₃)₂CO₂, benzoate, naphthylate, tosylate, mesylate, or trifluoromethane-sulfonate. In one more preferred embodiment, X¹ is nitrate (NO₃⁻).

In certain catalysts, R¹ is hydrogen and R² is selected from C₁-C₂₀ alkyl, C₂-C₂₀ alkenyl, and C₅-C₂₄ aryl, more preferably C₁-C₆ alkyl, C₂-C₆ alkenyl, and C₅-C₁₄ aryl. Still more preferably, R² is phenyl, vinyl, methyl, isopropyl, or t-butyl, optionally substituted with one or more moieties selected from C₁-C₆ alkyl, C₁-C₆ alkoxy, and phenyl. Most preferably, R² is phenyl or vinyl substituted with one or more moieties selected from methyl, ethyl, chloro, bromo, iodo, fluoro, nitro, dimethylamino, methyl, methoxy, and phenyl. More specifically, R² may be phenyl or —C═C(CH₃)₂.

Any two or more (typically two, three, or four) of X¹, L¹, R¹, and R² can be taken together to form a cyclic group, including bidentate or multidentate ligands, as disclosed, for example, in U.S. Pat. No. 5,312,940 to Grubbs et al. When any of X¹, L¹, R¹, and R² are linked to form cyclic groups, those cyclic groups may contain 4 to 12, preferably 4, 5, 6, 7 or 8 atoms, or may comprise two or three of such rings, which may be either fused or linked.

In particular embodiments, Q is a two-atom linkage having the structure —CR¹¹R¹²—CR¹³R¹⁴— or —CR¹¹═CR¹³—, preferably —CR¹¹R¹²—CR¹³R¹⁴—, wherein R¹¹, R¹², R¹³, and R¹⁴ are independently selected from hydrogen, hydrocarbyl, substituted hydrocarbyl, heteroatom-containing hydrocarbyl, substituted heteroatom-containing hydrocarbyl, and functional groups. Examples of suitable functional groups include carboxyl, C₁-C₂₀ alkoxy, C₅-C₂₄ aryloxy, C₂-C₂₀ alkoxycarbonyl, C₅-C₂₄ alkoxycarbonyl, C₂-C₂₄ acyloxy, C₁-C₂₀ alkylthio, C₅-C₂₄ arylthio, C₁-C₂₀ alkylsulfonyl, and C₁-C₂₀ alkylsulfinyl, optionally substituted with one or more moieties selected from C₁-C₁₂ alkyl, C₁-C₁₂ alkoxy, C₅-C₁₄ aryl, hydroxyl, sulfhydryl, formyl, and halide. R¹¹, R¹², R¹³, and R¹⁴ are preferably independently selected from hydrogen, C₁-C₁₂ alkyl, substituted C₁-C₁₂ alkyl, C₁-C₁₂ heteroalkyl, substituted C₁-C₁₂ heteroalkyl, phenyl, and substituted phenyl. Alternatively, any two of R¹¹, R¹², R¹³, and R¹⁴ may be linked together to form a substituted or unsubstituted, saturated or unsaturated ring structure, e.g., a C₄-C₁₂ alicyclic group or a C₅ or C₆ aryl group, which may itself be substituted, e.g., with linked or fused alicyclic or aromatic groups, or with other substituents. In one further aspect, any one or more of R¹¹, R¹², R¹³, and R¹⁴ comprises one or more of the linkers.

In more particular aspects, R³ and R⁴ maybe alkyl or aryl, and may be independently selected from alkyl, aryl, cycloalkyl, heteroalkyl, alkenyl, alkynyl, and halo or halogen-containing groups. More specifically, R³ and R⁴ may be independently selected from C₁-C₂₀ alkyl, C₅-C₁₄ cycloalkyl, C₁-C₂₀ heteroalkyl, or halide. Suitable alkyl groups include, without limitation, methyl, ethyl, n-propyl, isopropyl, isopropyl, n-butyl, isobutyl, t-butyl, octyl, decyl, and the like; suitable cycloalkyl groups include cyclopentyl, cyclohexyl, adamantyl, pinenyl, terpenes and terpenoid derivatives and the like; suitable alkenyl groups include ethenyl, n-propenyl, isopropenyl, n-butenyl, isobutenyl, octenyl, decenyl, tetradecenyl, hexadecenyl, eicosenyl, tetracosenyl, and the like; suitable alkynyl groups include ethynyl, n-propynyl, and the like.

When R³ or R⁴ are aromatic, each can be independently composed of one or two aromatic rings, which may or may not be substituted, e.g., R³ and R⁴ may be phenyl, substituted phenyl, biphenyl, substituted biphenyl, or the like. In a particular embodiment, R³ and R⁴ are independently an unsubstituted phenyl or phenyl substituted with up to three substituents selected from C₁-C₂₀ alkyl, C₁-C₂₀ alkylcarboxylate, substituted C₁-C₂₀ alkyl, C₁-C₂₀ heteroalkyl, substituted C₁-C₂₀ heteroalkyl, C₅-C₂₄ aryl, substituted C₅-C₂₄ aryl, C₅-C₂₄ heteroaryl, C₆-C₂₄ aralkyl, C₆-C₂₄ alkaryl, or halide. Preferably, any substituents present are hydrogen C₁-C₁₂ alkyl, C₁-C₁₂ alkoxy, C₅-C₁₄ aryl, substituted, C₅-C₁₄ aryl, or halide. More particularly, R³ and R⁴ may be independently substituted with hydrogen, C₁-C₄ alkyl, C₁-C₄ alkylcarboxylate, C₁-C₄ alkoxy, C₅-C₁₄ aryl, substituted C₅-C₁₄ aryl, or halide. As an example, R³ and R⁴ are selected from cyclopentyl, cyclohexyl, adamantyl, norbonenyl, pinenyl, terpenes and terpenoid derivatives, mesityl, diisopropylphenyl or, more generally, cycloalkyl substituted with one, two or three C₁-C₄ alkyl or C₁-C₄ alkoxy groups, or a combination thereof.

Particular complexes wherein R² and L¹ are linked to form a chelating carbene ligand are examples of another group of catalysts, and are commonly called “Grubbs-Hoveyda” catalysts. Grubbs-Hoveyda metathesis-active metal carbene complexes of the invention may be described by the formula VIII.

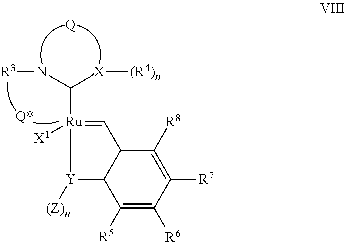

wherein,

X¹, Q, Q*, R³ and R⁴ are as previously defined herein;

Y is a heteroatom selected from N, O, S, and P; preferably Y is O or N;

R⁵, R⁶, R⁷, and R⁸ are each, independently, selected from the group consisting of hydrogen, halogen, alkyl, alkenyl, alkynyl, aryl, heteroalkyl, heteroatom containing alkenyl, heteroalkenyl, heteroaryl, alkoxy, alkenyloxy, aryloxy, alkoxycarbonyl, carbonyl, alkylamino, alkylthio, aminosulfonyl, monoalkylaminosulfonyl, dialkylaminosulfonyl, alkylsulfonyl, nitrile, nitro, alkylsulfinyl, trihaloalkyl, perfluoroalkyl, carboxylic acid, ketone, aldehyde, nitrate, cyano, isocyanate, hydroxyl, ester, ether, amine, imine, amide, halogen-substituted amide, trifluoroamide, sulfide, disulfide, sulfonate, carbamate, silane, siloxane, phosphine, phosphate, or borate, wherein any combination of R⁵, R⁶, R⁷, and R⁸ can be linked to form one or more cyclic groups;
n is 1 or 2, such that n is 1 for the divalent heteroatoms O or S, and n is 2 for the trivalent heteroatoms N or P; and

Z is a group selected from hydrogen, alkyl, aryl, functionalized alkyl, functionalized aryl where the functional group(s) may independently be one or more or the following: alkoxy, aryloxy, halogen, carboxylic acid, ketone, aldehyde, nitrate, cyano, isocyanate, hydroxyl, ester, ether, amine, imine, amide, trifluoroamide, sulfide, disulfide, carbamate, silane, siloxane, phosphine, phosphate, or borate; methyl, isopropyl, sec-butyl, t-butyl, neopentyl, benzyl, phenyl and trimethylsilyl; and wherein any combination or combinations of X¹, Q*, Y, Z, R⁵, R⁶, R⁷, and R⁸ are linked to a support.

### Strained Olefin Reactant

The AROCM reaction catalyzed by the complexes described above involve a strained olefin reactant and a second α-olefin reactant, wherein the two reactants are brought into contact in the presence of a catalytically effective amount of the complex, under conditions and for a time period effective to allow the AROCM reaction to occur. In general, the strained olefin reactant may be represented by the structure of formula (XIII):

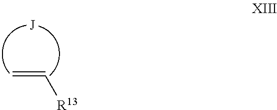

wherein J and R¹³ are as follows:

R¹³ is selected from the group consisting of hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), and substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl) and, if substituted hydrocarbyl or substituted heteroatom-containing hydrocarbyl, wherein the substituents may be functional groups (“Fn”) such as phosphonato, phosphoryl, phosphanyl, phosphino, sulfonato, C₁-C₂₀ alkylsulfanyl, C₅-C₂₀ arylsulfanyl, C₁-C₂₀ alkylsulfonyl, C₅-C₂₀ arylsulfonyl, C₁-C₂₀ alkylsulfinyl, C₅-C₂₀ arylsulfinyl, sulfonamido, amino, amido, imino, nitro, nitroso, hydroxyl, C₁-C₂₀ alkoxy, C₅-C₂₀ aryloxy, C₂-C₂₀ alkoxycarbonyl, C₅-C₂₀ aryloxycarbonyl, carboxyl, carboxylato, mercapto, formyl, C₁-C₂₀ thioester, cyano, cyanato, carbamoyl, epoxy, styrenyl, silyl, silyloxy, silanyl, siloxazanyl, boronato, boryl, or halogen, or a metal-containing or metalloid-containing group (wherein the metal may be, for example, Sn or Ge). R¹³ may itself be one of the aforementioned groups, such that the Fn moiety is directly bound to the olefinic carbon atom indicated in the structure. In the latter case, however, the functional group will generally not be directly bound to the olefinic carbon through a heteroatom containing one or more lone pairs of electrons, e.g., an oxygen, sulfur, nitrogen or phosphorus atom, or through an electron-rich metal or metalloid such as Ge, Sn, As, Sb, Se, Te, etc. With such functional groups, there will normally be an intervening linkage Z, such that R¹³ then has the structure —(Z)ₙ-Fn wherein n is 1, Fn is the functional group, and Z is a hydrocarbylene linking group such as an alkylene, substituted alkylene, heteroalkylene, substituted heteroalkene, arylene, substituted arylene, heteroarylene, or substituted heteroarylene linkage.

J is a saturated or unsaturated hydrocarbylene, substituted hydrocarbylene, heteroatom-containing hydrocarbylene, or substituted heteroatom-containing hydrocarbylene linkage, wherein when J is substituted hydrocarbylene or substituted heteroatom-containing hydrocarbylene, the substituents may include one or more —(Z)ₙ—Fn groups, wherein n is zero or 1, and Fn and Z are as defined previously. Additionally, two or more substituents attached to ring carbon (or other) atoms within J may be linked to form a bicyclic or polycyclic olefin. J will generally contain in the range of approximately 4 to 14 ring atoms, typically 4 to 8 ring atoms, for a monocyclic olefin, and, for bicyclic and polycyclic olefins, each ring will generally contain 4 to 8, typically 5 to 7, ring atoms.

Mono-unsaturated cyclic olefin reactants encompassed by structure (XII) may be represented by the structure (XIV):

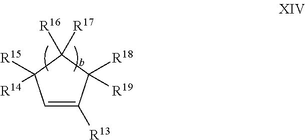

wherein b is an integer generally although not necessarily in the range of 0 to 10, typically 0 to 5, R¹³ is as defined above, and R¹⁴, R¹⁵, R¹⁶, R¹⁷, R¹⁸, and R¹⁹ are independently selected from the group consisting of hydrogen, hydrocarbyl, substituted hydrocarbyl, heteroatom-containing hydrocarbyl, substituted heteroatom-containing hydrocarbyl and —(Z)ₙ-Fn where n, Z and Fn are as defined previously, and wherein if any of the R¹⁴ through R¹⁹ moieties is substituted hydrocarbyl or substituted heteroatom-containing hydrocarbyl, the substituents may include one or more —(Z)ₙ-Fn groups. Accordingly, R¹⁴, R¹⁵, R¹⁶, R¹⁷, R¹⁸, and R¹⁹ may be, for example, hydrogen, hydroxyl, C₁-C₂₀ alkyl, C₅-C₂₀ aryl, C₁-C₂₀ alkoxy, C₅-C₂₀ aryloxy, C₂-C₂₀ alkoxycarbonyl, C₅-C₂₀ aryloxycarbonyl, amino, amido, nitro, etc. Furthermore, any of the R¹⁴ through R¹⁹ moieties can be linked to any other of the R¹⁴ through R¹⁹ moieties to provide a bicyclic or polycyclic olefin, and the linkage may include heteroatoms or functional groups, e.g., the linkage may include an ether, ester, thioether, amino, alkylamino, imino, or anhydride moiety.

Examples of monounsaturated, monocyclic olefins encompassed by structure (XIV) include, without limitation, cyclopentene, cyclohexene, cycloheptene, cyclooctene, cyclononene, cyclodecene, cycloundecene, cyclododecene, tricyclodecene, tetracyclodecene, octacyclodecene, and cycloeicosene, and substituted versions thereof such as 1-methylcyclopentene, 1-ethylcyclopentene, 1-isopropylcyclohexene, 1-chloropentene, 1-fluorocyclopentene, 1-methylcyclopentene, 4-methoxy-cyclopentene, 4-ethoxy-cyclopentene, cyclopent-3-ene-thiol, cyclopent-3-ene, 4-methylsulfanyl-cyclopentene, 3-methylcyclohexene, 1-methylcyclooctene, 1,5-dimethylcyclooctene, etc.

Monocyclic diene reactants encompassed by structure (XIII) may be generally represented by the structure (XV):

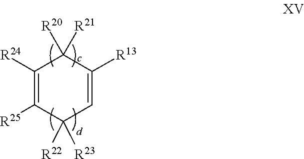

wherein c and d are independently integers in the range of 1 to about 8, typically 2 to 4, preferably 2 (such that the reactant is a cyclooctadiene), R¹³ is as defined above, and R²⁰, R²¹, R²², R²³, R²⁴ and R²⁵ are defined as for R¹⁴ through R¹⁹. In this case, it is preferred that R²⁴ and R²⁵ be nonhydrogen substituents, in which case the second olefinic moiety is tetrasubstituted, so that the ROCM reaction proceeds selectively at only one of the two olefin functionalities. Examples of monocyclic diene reactants include, without limitation, 1,3-cyclopentadiene, 1,3-cyclohexadiene, 1,3-cyclohexadiene, 5-ethyl-1,3-cyclohexadiene, 1,3-cycloheptadiene, cyclohexadiene, 1,5-cyclooctadiene, 1,3-cyclooctadiene, and substituted analogs thereof. Triene reactants are analogous to the diene structure (XV), and will generally contain at least one methylene linkage between any two olefinic segments.

Bicyclic and polycyclic olefinic reactants encompassed by structure (XII) may be generally represented by the structure (XVI)

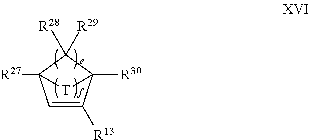

wherein e is an integer in the range of 1 to 8, typically 2 to 4, f is generally 1 or 2, T is lower alkylene or lower alkenylene, generally substituted or unsubstituted methyl or ethyl, R¹³ is as defined above, and R²⁷, R²⁸, R²⁹, and R³⁰ are as defined for R¹⁴ through R¹⁹. Preferred olefinic reactants within this group are in the norbornene family, having the structure (XVII)

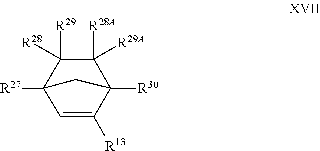

wherein R¹³, and R²⁷ through R³⁰ are as defined previously, and R^{28A} and R^{29A} are defined as for R²⁸ and R²⁹.

Examples of bicyclic and polycyclic olefinic reactants thus include, without limitation, dicyclopentadiene, tricyclopentadiene, dicyclohexadiene, norbornene, 5-methyl-2-norbornene, 5-ethyl-2-norbornene, 5-isobutyl-2-norbornene, 5,6-dimethyl-2-norbornene, 5-phenylnorbornene, 5-benzylnorbornene, 5-acetylnorbornene, 5-methoxycarbonylnorbornene, 5-ethoxycarbonylnorbornene, 5-methyl-5-methoxy-carbonylnorbornene, 5-cyanonorbornene, 5,5,6-trimethyl-2-norbornene, cyclo-hexenylnorbornene, endo, exo-5,6-dimethoxynorbornene, endo, endo-5,6-dimethoxynorbornene, endo,exo-5,6-dimethoxycarbonyl-norbornene, endo, endo-5,6-dimethoxycarbonylnorbornene, 2,3-dimethoxynorbornene, norbornadiene, tricycloundecene, tetracyclododecene, 8-methyltetracyclododecene, 8-ethyl-tetracyclododecene, 8-methoxycarbonyltetracyclododecene, 8-methyl-8-tetracyclo-dodecene, 8-cyanotetracyclododecene, pentacyclopentadecene, pentacyclohexadecene, 1,9-octadecadiene, and the like.

### α-Olefin Reactant

In general, the α-olefin reactant may be represented by the structure of formula (XVIII):

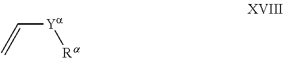

wherein Y^{α} is selected from the group comprising nil, CH₂, O, or S and R^{α} is selected from the group consisting of hydrogen, hydrocarbyl (e.g., C₁-C₂₀ alkyl, C₅-C₂₀ aryl, C₅-C₃₀ aralkyl, or C₅-C₃₀ alkaryl), substituted hydrocarbyl (e.g., substituted C₁-C₂₀ alkyl, C₅-C₂₀ aryl, C₅-C₃₀ aralkyl, or C₅-C₃₀ alkaryl), heteroatom-containing hydrocarbyl (e.g., C₁-C₂₀ heteroalkyl, C₅-C₂₀ heteroaryl, heteroatom-containing C₅-C₃₀ aralkyl, or heteroatom-containing C₅-C₃₀ alkaryl), and substituted heteroatom-containing hydrocarbyl (e.g., substituted C₁-C₂₀ heteroalkyl, C₅-C₂₀ heteroaryl, heteroatom-containing C₅-C₃₀ aralkyl, or heteroatom-containing C₅-C₃₀ alkaryl) and, if substituted hydrocarbyl or substituted heteroatom-containing hydrocarbyl, wherein the substituents may be functional groups (“Fn”) such as phosphonato, phosphoryl, phosphanyl, phosphino, sulfonato, C₁-C₂₀ alkylsulfanyl, C₅-C₂₀ arylsulfanyl, C₁-C₂₀ alkylsulfonyl, C₅-C₂₀ arylsulfonyl, C₁-C₂₀ alkylsulfinyl, C₅-C₂₀ arylsulfinyl, sulfonamido, amino, amido, imino, nitro, nitroso, hydroxyl, C₁-C₂₀ alkoxy, C₅-C₂₀ aryloxy, C₂-C₂₀ alkoxycarbonyl, C₅-C₂₀ aryloxycarbonyl, carboxyl, carboxylato, mercapto, formyl, C₁-C₂₀ thioester, cyano, cyanato, carbamoyl, epoxy, styrenyl, silyl, silyloxy, silanyl, siloxazanyl, boronato, boryl, or halogen, or a metal-containing or metalloid-containing group (wherein the metal may be, for example, Sn or Ge).

### Catalyzed Asymmetric Ring Opening/Cross Metathesis of Bicyclic Olefins and α-Olefins

We anticipated that enantiopure versions of the newly developed catalysts would exhibit high Z-selectivity and enantioselectivity in AROCM due to the rigidity imparted by the Ru—C chelate. Herein we report a new homochiral stereogenic-at-ruthenium complex that exhibits high enantioselectivity in the AROCM of norbornene derivatives.

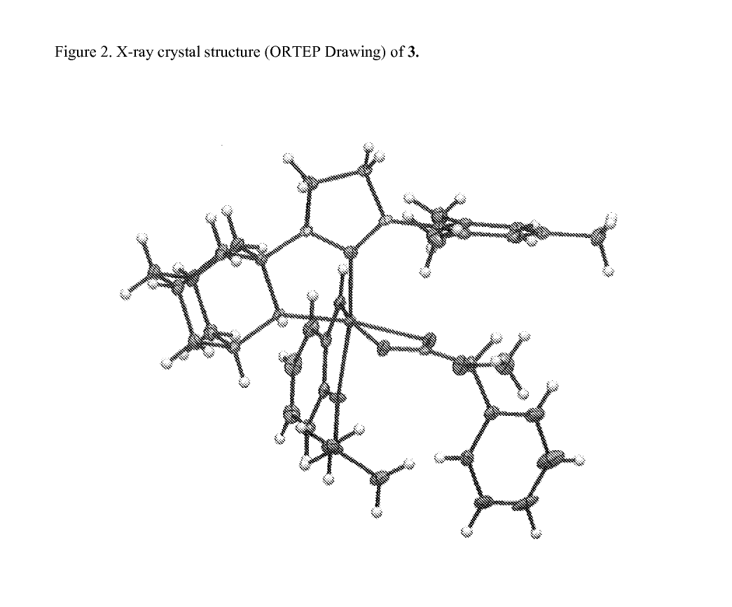

Enantioenriched 4 was synthesized by resolution as shown in Scheme 1. Treatment of racemic iodide 1 (see Keitz, B. K.; Endo, K.; Patel, P. R.; Herbert, M. B.; Grubbs, R. H., J. Am. Chem. Soc. 2012, 134, 693-699) with silver carboxylate 2 cleanly formed a 1:1 mixture of diastereomers in 97% yield. Chromatographic separation of the mixture afforded a 45% yield (90% of theoretical maximum) of 3 (>95:5 dr). The absolute stereochemistry of complex 3 was confirmed by X-ray crystallography (FIG. 2). Sequential treatment of carboxylate 3 with para-toluenesulfonic acid and sodium nitrate produced the enantioenriched nitrate complex 4 in 43% yield.

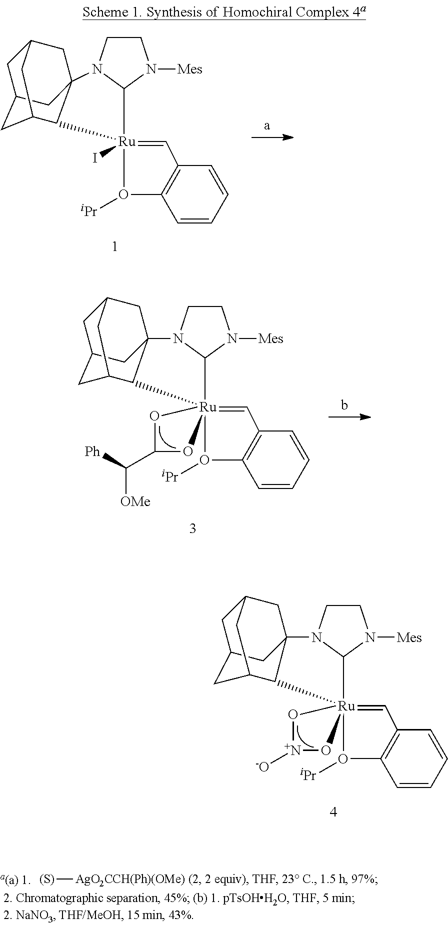

While complex 3 exhibited low enantioselectivity in AROCM, 1 mol % of complex 4 catalyzed the reaction of norbornene 5 with an excess of allyl acetate (6) to produce a 64% yield of diene (1S,2R,3S,4R)-7 with 95% Z-selectivity and 93% ee (Scheme 2) (Absolute configurations were assigned by analogy to that of 9c, which was determined by X-ray crystallography). The highly selective reaction produces four contiguous stereocenters on a tetra-substituted cyclopentane ring. Optimization of the process revealed that 7 equiv. of α-olefin, 1 mol % catalyst loading at 23° C. and 0.5 M concentration in THF afforded the highest yield and selectivity. Ethereal solvents were optimal, with catalyst solubility improved in THF over diethyl ether.

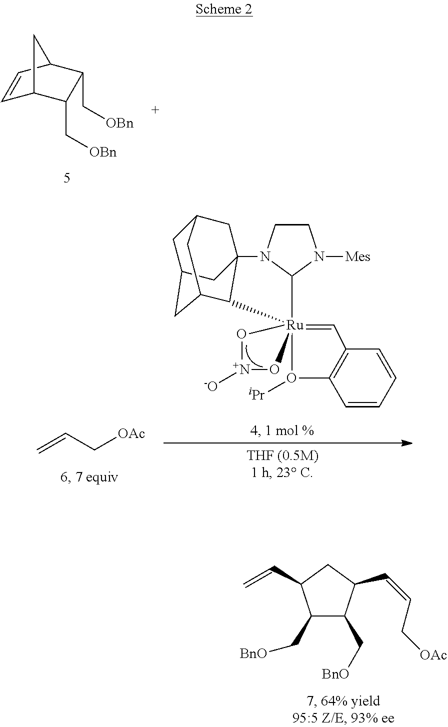

In order to demonstrate the scope of Z-selective catalyst 4, a variety of α-olefins bearing diverse functionality were employed in order to determine their effect on the efficiency and enantioselectivity of the reaction. As illustrated in Table 1, replacing allyl acetate with N-Boc-allylamine provided amine-containing product 8a in equally high enantioselectivity (94% ee). Utilizing an olefin bearing a remote ester did not impact the Z-selectivity and afforded 8b in 91% ee.

TABLE 1
AROCM with Different α-Olefin Partners^{a}

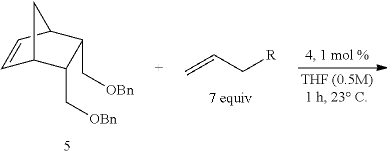

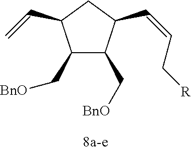

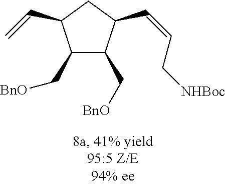 [SMILES: C=C[C@H]1C[C@@H](/C=C\CNC(=O)OC(C)(C)C)[C@@H](COCc2ccccc2)[C@H]1COCc1ccccc1]

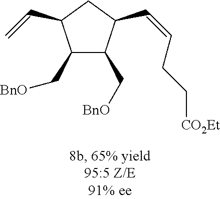 [SMILES: C=C[C@H]1C[C@@H](/C=C\CCC(=O)OCC)[C@@H](COCc2ccccc2)[C@H]1COCc1ccccc1]

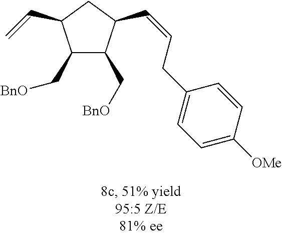 [SMILES: C=C[C@H]1C[C@@H](/C=C\Cc2ccc(OC)cc2)[C@@H](COCc2ccccc2)[C@H]1COCc1ccccc1]

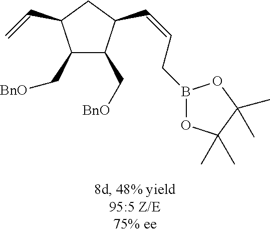 [SMILES: C=C[C@H]1C[C@@H](/C=C\CB2OC(C)(C)C(C)(C)O2)[C@@H](COCc2ccccc2)[C@H]1COCc1ccccc1]

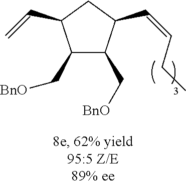

^{a}Yields correspond to isolated product; Z/E ratios determined by 500 MHz ¹H NMR of the crude reaction mixture; ee of pure products measured with chiral SFC.

Bulkier allylic substituents such as para-methoxy phenyl and pinacol boronic ester gave products 8c and 8d with moderate enantioselectivity (81% and 75% ee, respectively). A simple α-olefin such as 1-hexene also gave good yield, Z-selectivity, and enantioselectivity (8e, 89% ee), demonstrating that allylic functionality is not required to confer a selective reaction. The examples in Table 1 suggest that catalyst 4 is capable of producing a range of AROCM products (Attempts to employ heteroatom-substituted olefins (butyl vinyl ether) resulted in no ROCM product, presumably due to catalyst deactivation).

The norbornene component was then altered to understand its impact on Z-selectivity and enantioselectivity. As a basis for comparison, the substrates were treated with 7 equivalents of allyl acetate under the optimized catalytic conditions. Norbornenes bearing coordinating functionality such as acetate (to form 9a) and N-phenyl succinimide (to form 9b) resulted in reduced yield and slower reaction, respectively. The dimethyl substituted anhydride afforded a 65% yield of 9d, which contains two vicinal all-carbon quaternary stereocenters, demonstrating the power of AROCM to afford otherwise synthetically challenging products in high ee (95%). Aryl ether 9e was produced in 95% ee, although interestingly as a 7:3 Z/E mixture. The results in Table 2 support the observation that substrates bearing 2,3-endo substitution react with high Z-selectivity; substrates lacking this substitution pattern show reduced diastereoselectivity.

TABLE 2
Influence of Strained Olefin Reactant^{a}

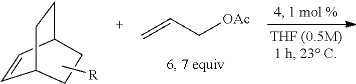

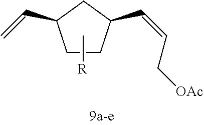

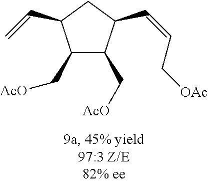 [SMILES: C=C[C@H]1C[C@@H](/C=C\COC(C)=O)[C@@H](COC(C)=O)[C@H]1COC(C)=O]

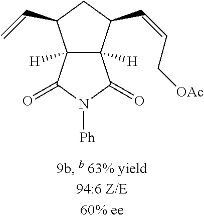 [SMILES: C=C[C@H]1C[C@@H](/C=C\COC(C)=O)[C@H]2C(=O)N(c3ccccc3)C(=O)[C@H]21]

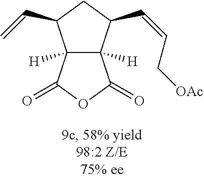 [SMILES: C=C[C@H]1C[C@@H](/C=C\COC(C)=O)[C@H]2C(=O)OC(=O)[C@H]21]

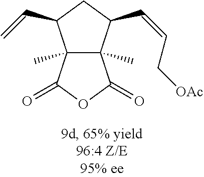 [SMILES: C=C[C@H]1C[C@@H](/C=C\COC(C)=O)[C@@]2(C)C(=O)OC(=O)[C@@]12C]

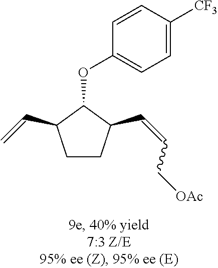 [SMILES: C=C[C@@H]1CC[C@H](C=CCOC(C)=O)[C@H]1Oc1ccc(C(F)(F)F)cc1]

^{a}Yields correspond to isolated product; Z/E ratios determined by GC; ee of pure products measured with chiral SFC.
^{b} Conducted at 3 mol % catalyst loading for 5 h.

The fact that Z-9e and E-9e are formed in identical enantioenrichment has important mechanistic implications and offers indirect evidence of the active catalytic species. The result suggests that the enantiodetermining step most likely precedes the olefin geometry-determining step (This assumes that secondary metathesis processes proceed at a negligible rate compared to the productive (ROCM) reaction. Measurements of the formation of 9e (see Experimental) show that the Z/E ratio is constant during the course of the reaction and for several hours after complete conversion). This conclusion requires the initial enantiodetermining ring-opening event to occur with a ruthenium methylidene (Scheme 3). Subsequent cross metathesis of the ring-opened product bearing a ruthenium alkylidene with an equivalent of α-olefin would then produce the observed product.

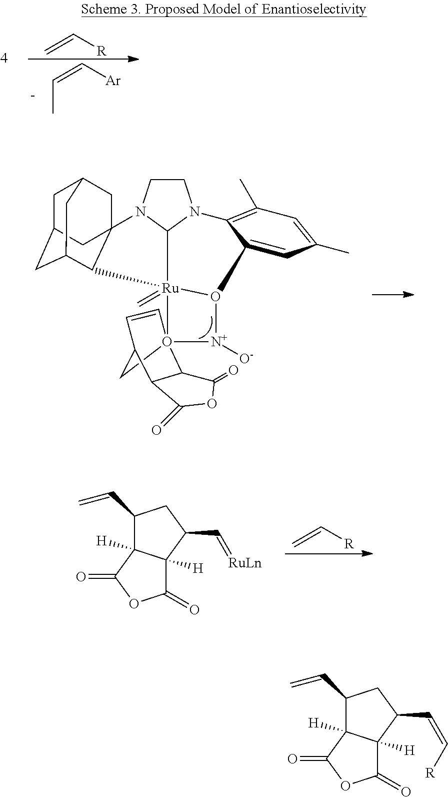

On the basis of this indirect mechanistic evidence and the absolute configuration of the isolated product, we propose that the methylidene shown in Scheme 3 initially reacts with the norbornene component in an enantioselective ring-opening event. It is hypothesized that the enantioselectivity is governed by approach of the methylidene to the less-hindered exo face while the mesityl “cap” forces the bulk of the norbornene component to orient away from the NHC ligand (see Liu, P.; Xu, X.; Dong, X.; Keitz, B. K.; Herbert, M. B.; Grubbs, R. H.; Houk, K. N., J. Am. Chem. Soc. 2012, 134, 1464-1467). The proposed methylidene is most likely produced by initial cross metathesis of 4 with a molecule of α-olefin, resulting in epimerization at the ruthenium center.

### Catalyzed Asymmetric Ring Opening/Cross Metathesis of Monocyclic Olefins and α-Olefins

It was envisioned that the desymmetrization of suitably substituted meso cyclobutenes in AROCM would afford the 1,2-anti diol motif in perfect anti diastereoselectivity and potentially high enantioselectivity upon application of a newly developed cyclometalated metathesis catalyst 4 (Scheme 4) (see J. Hartung, R. H. Grubbs, J. Am. Chem. Soc. 2013, 135, 10183-10185). The resultant 1,5-diene would be a versatile synthetic intermediate due to the differential reactivity of the two alkenes, paving the way for further chemoselective transformations. Herein, we report the successful application of 4 to afford highly enantioenriched 1,2-anti diols and demonstrate the versatility of these products in the synthesis of the insect pheromone (+)-endo brevicomin and a derivative of the monosaccharide L-ribose. Pest control strategies utilizing insect pheromones have become a promising alternative to the application of broad-spectrum insecticides, underscoring the importance of rapid synthetic routes to (+)-endo brevicomin and related bioactive compounds (see (a) P. E. Howse, I. D. R. Stevens, Insect Pheromones and their Use in Pest Management, Chapman & Hall, New York, 1998; (b) Recent work from this group has demonstrated the application of racemic 4 to the synthesis of Lepidoptera female sex pheromones, see M. B. Herbert, V. M. Marx, R. L. Pederson, R. H. Grubbs, Angew. Chem. 2013, 125, 328-332; Angew. Chem., Int. Ed. 2013, 52, 310-314).

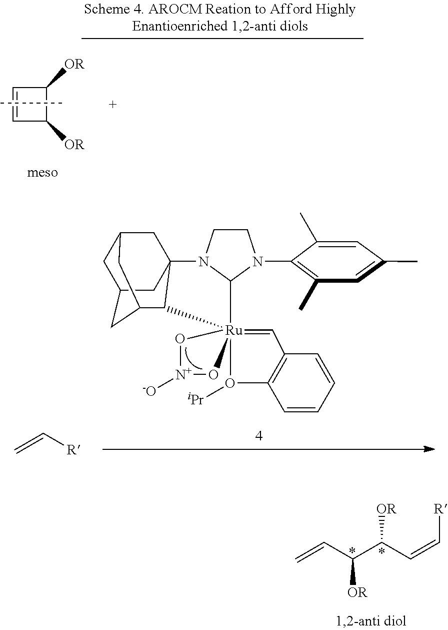

Initial attempts to form 1,2-anti diols were carried out with complex 4, allyl acetate (11), and cis-3,4-dibenzyloxycyclobutene (10, Table 3), which was synthesized by substitution of commercially available cis-3,4-dichlorocyclobutene with sodium phenylmethanolate (see W. Kirmse, F. Scheidt, H. J. Vater, J. Am. Chem. Soc. 1978, 100, 3945-3946). Solvent had no effect on selectivity of the AROCM reaction except for slightly diminished enantioselectivity in CH₂Cl₂ (entry 1, Table 3); yield was highest in THF (entry 4, Table 3). The effect of stoichiometry in AROCM has been explored for a number of catalysts (see (a) J. M. Berlin, S. D. Goldberg, R. H. Grubbs, Angew. Chem. 2006, 118, 7753-7757; Angew. Chem., Int. Ed. 2006, 45, 7591-7595; (b) M. Yu, I. Ibrahem, M. Hasegawa, R. R. Schrock, A. H. Hoveyda, J. Am. Chem. Soc. 2012, 134, 2788-2799; (c) D. S. La, J. G. Ford, E. S. Sattely, P. J. Bonitatebus, R. R. Schrock, A. H. Hoveyda, J. Am. Chem. Soc. 1999, 121, 11603-11604). In the current study, an excess of terminal olefin was optimal (7 equiv, entry 4, Table 3); as the equivalents of terminal olefin were reduced, the yield of the reaction dropped, yet a modest yield of 29% could be obtained with 1.2 equivalents of 11. No di-cross products were observed. Reducing the concentration also resulted in lower yield, leading to the optimal conditions of 7 equiv. of terminal olefin 11 in THF at a concentration of 0.5 M in 10 with 1 mol % 4 for 1.5 h. It is worth noting that although alternative solvents or stoichiometry negatively impacted reaction efficiency, the diastereo- and enantioselectivity remained consistently high, demonstrating the robustness of the reaction.

TABLE 3
Optimization of the AROCM of Cyclobutene 10 with 11.

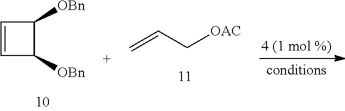

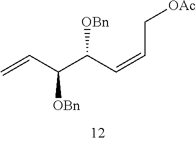 [SMILES: C=C[C@H](OCc1ccccc1)[C@@H](/C=C\COC(C)=O)OCc1ccccc1]

| | | Conc | | | | ee |
|---|---|---|---|---|---|---|
| entry | Equiv 11 | (M) | Solvent | Yield^{[a]} | % Z^{[a]} | (Z)^{[b]} |
| 1 | 7 | 0.5 | CH₂Cl₂ | 35 | 83 | 93 |
| 2 | 7 | 0.5 | Benzene | 49 | 84 | 95 |
| 3 | 7 | 0.5 | Toluene | 52 | 84 | 95 |
| 4 | 7 | 0.5 | THF | 79 | 85 | 95 |
| 5 | 5 | 0.5 | THF | 71 | 84 | 96 |
| 6 | 3 | 0.5 | THF | 63 | 85 | 96 |
| 7 | 1.2 | 0.5 | THF | 29 | 87 | 97 |
| 8 | 7 | 0.3 | THF | 72 | 85 | 95 |
| 9 | 7 | 0.1 | THF | 43 | 85 | 95 |

^{[a]}Determined by GC.
^{[b]}Determined by chiral SFC.

While the synthesis of a 1,2-anti alkoxy motif had been demonstrated, inclusion of alternative protecting groups on the diol motif strengthens the synthetic protocol. These modifications would allow a synthetic sequence to be designed taking into account the feasibility of removing the protecting groups in the presence of other functionality. Moreover, modulation of the size and electronics of the groups on the cyclobutene and terminal olefin reactants would provide a better understanding of the factors contributing to selectivity.

A complement of commonly used hydroxyl protecting groups were tolerated on the cyclobutene and terminal olefin reactants, but enantio- and diastereoselectivity were affected by the choice of substituents (Tables 4 and 5) (Attempts to use cyclic protecting groups (ex: benzylidene acetal) resulted in low conversion). The increased bulkiness of the tert-butyldimethylsilyl ether resulted in improved Z selectivity and remarkable enantioselectivity (88% Z, 99% ee, 15a, Table 4), while hydroxyls and benzoates on the cyclobutene reactant led to Z products with 91% and 96% ee, respectively. The same enantioinduction was observed in products 15a and 15b. Isopropoxy substituents on the cyclobutene resulted in abrogation of catalyst activity presumably due to the formation of a stable chelating complex (In preliminary stoichiometric experiments with 4 and 13c, we observe the formation of a kinetically stable intermediate analogous to one described in a recent report on an enantiopure ruthenium alkylidene complex, see R. K. M. Khan, A. R. Zhugralin, S. Torker, R. V. O'Brien, P. J. Lombardi, A. H. Hoveyda, J. Am. Chem. Soc. 2012, 134, 12438-12441).

TABLE 4
Scope of the AROCM Reaction with respect to Cyclobutene Substitution.^{[a]}

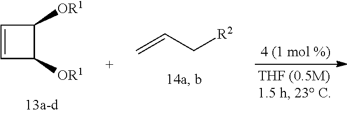

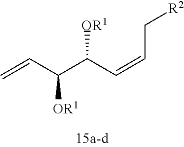

| | | | | % | ee Z^{[d]} |
|---|---|---|---|---|---|
| R¹ | R² | Product | Yield^{[b]} | Z^{[c]} | (ee E)^{[d]} |
| TBS | OH | 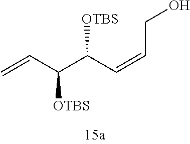 | 66 | 88 | 99 (nd) |
| H | OBz | 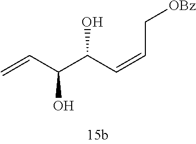 | 67 | 75 | 91 (67) |
| Bz | OH | 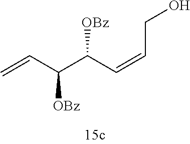 | 69 | 75 | 96 (82) |
| ⁱPr | OBz | 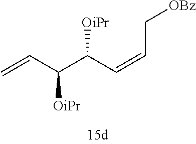 | <5 | nd | nd |

^{[a]}0.1 mmol cyclobutene, 0.7 mmol terminal olefin.
^{[b]}Combined isolated yield of E and Z products.
^{[c]}Determined by 500 MHz ¹H NMR analysis of crude reaction mixture.
^{[d]}Determined by chiral SFC.

High enantioselectivities were obtained with a wide range of terminal olefins. Among the 0-protecting groups surveyed (Table 5, 15e-h), the tert-butyldimethylsilyl group resulted in high enantioselectivity (89% ee, 15g), but the more electron-withdrawing benzoate ester was optimal, resulting in the highest enantioselectivity (97% ee, 15f). Terminal olefins bearing alkyl substitution resulted in higher diastereoselectivity and yield with similar levels of enantioselectivity (15i, 15j). The chiral allylation reagent 15k was synthesized in 91% ee, affording a functionally useful building block. Z and E isomers were isolable from each other by flash or thin layer chromatography in all cases except 15i.

TABLE 5
Scope of the AROCM Reaction with respect to Terminal Olefin^{[a]}

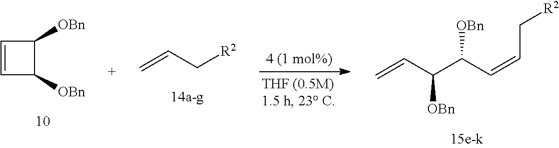

| | | | | ee Z^{[d]} |
|---|---|---|---|---|
| R² | Product | Yield^{[b]} | % Z^{[c]} | (ee E)^{[d]} |
| OH | 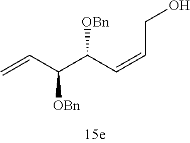 [SMILES: C=C[C@H](OCc1ccccc1)[C@@H](/C=C\CO)OCc1ccccc1] | 62 | 89 | 93 (86) |
| OBz | 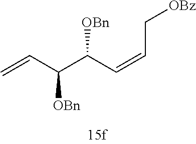 | 61 | 88 | 97 (88) |
| OTBS | 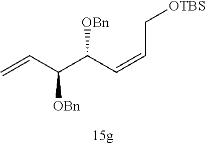 [SMILES: C=C[C@H](OCc1ccccc1)[C@@H](/C=C\CO[Si](C)(C)C(C)(C)C)OCc1ccccc1] | 68^{[e]} | 87 | 89 (77) |
| OBn | 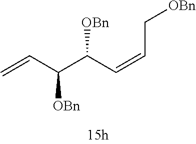 [SMILES: C=C[C@H](OCc1ccccc1)[C@@H](/C=C\COCc1ccccc1)OCc1ccccc1] | 64 | 86 | 91 (nd) |
| 4-MeOPh | 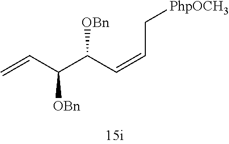 | 76 | 90 | 93 (79) |
| CH₂C(O)CH₃ | 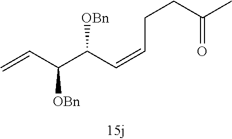 [SMILES: C=C[C@H](OCc1ccccc1)[C@@H](/C=C\CCC(C)=O)OCc1ccccc1] | 65 | 90 | 92 (84) |
| BPin | 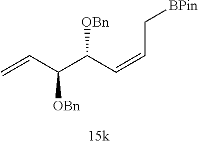 | 50 | nd^{[f]} | 91 (nd) |

^{[a]}0.1 mmol cyclobutene, 0.7 mmol terminal olefin.
^{[b]}Combined isolated yield of E and Z products.
^{[c]}Determined by 500 MHz ¹H NMR analysis of crude reaction mixture.
^{[d]}Determined by chiral SFC.
^{[e]}Yield determined after derivatization to 15e.
^{[f]}Not determined due to instability of E product.

We next explored the synthetic utility of the 1,2-anti diol fragments produced in the AROCM reaction. Cyclic ketals derived from the 1,2-anti diol motif feature prominently in the structures of several natural products (see (a) R. M. Silverstein, R. G. Brownlee, T. E. Bellas, D. L. Wood, L. E. Browne, Science 1968, 159, 889-891; (b) T. Yasumoto, M. Murata, Y. Oshima, M. Sano, G. K. Matsumoto, J. Clardy, Tetrahedron 1985, 41, 1019-1025; (c) D. Uemura, T. Chou, T. Haino, A. Nagatsu, S. Fukuzawa, S. Z. Zheng, H. S. Chen, J. Am. Chem. Soc. 1995, 117, 1155-1156; (d) T. Chou, O. Kamo, D. Uemura, Tetrahedron Lett. 1996, 37, 4023-4026; (e) T. Chou, T. Haino, M. Kuramoto, D. Uemura, Tetrahedron Lett. 1996, 37, 4027-4030). Accordingly, we targeted this structure in the context of a synthesis of the insect pheromone (+)-endo brevicomin (19, Scheme 5) (For catalytic asymmetric syntheses, see (a) A. C. Oehlschlager, B. D. Johnston, J. Org. Chem. 1987, 52, 940-943; (b) S. D. Burke, N. Muller, C. M. Beaudry, Org. Lett. 1999, 1, 1827-1829; (c) S.-G. Kim, T.-H. Park, B. J. Kim, Tetrahedron Lett. 2006, 47, 6369-6372; (d) S. Singh, P. J. Guiry, J. Org. Chem. 2009, 74, 5758-5761; for syntheses relying on stoichiometric chiral reagents, see (e) R. Bernardi, C. Fuganti, P. Grasselli, Tetrahedron Lett. 1981, 22, 4021-4024; (f) K. Mori, Y. B. Seu, Tetrahedron 1985, 41, 3429-3431; (g) F. Sato, O. Takahashi, T. Kato, Y. Kobayashi, J. Chem. Soc., Chem. Commun. 1985, 1638-1641; (h) S. Hatakeyama, K. Sakurai, S. Takano, J. Chem. Soc., Chem. Commun. 1985, 1759-1761; (i) A. Yusufoglu, S. Antons, H. D. Scharf, J. Org. Chem. 1986, 51, 3485-3487; (j) J. Mulzer, A. Angermann, W. Munch, Liebigs Ann. Chem. 1986, 825-838; (k) H. Redlich, W. Bruns, W. Francke, V. Schurig, T. L. Payne, J. P. Vite, Tetrahedron 1987, 43, 2029-2034; (l) J. M. Chong, E. K. Mar, Tetrahedron 1989, 45, 7709-7716; (m) Y. Noda, M. Kikuchi, Chem. Lett. 1989, 1755-1756; (n) S. Ramaswamy, A. C. Oehlschlager, J. Org. Chem. 1989, 54, 255-257; (o) K. Matsumoto, N. Suzuki, H. Ohta, Tetrahedron Lett. 1990, 31, 7163-7166; (p) G. Pedrocchifantoni, S. Servi, J. Chem. Soc., Perkin 1 1991, 1764-1765; (q) V. Cere, C. Mazzini, C. Paolucci, S. Pollicino, A. Fava, J. Org. Chem. 1993, 58, 4567-4571; (r) J. A. Soderquist, A. M. Rane, Tetrahedron Lett. 1993, 34, 5031-5034; (s) A. Gypser, M. Flasche, H. D. Scharf, Liebigs Ann. Chem. 1994, 775-780; (t) M. J. Kim, G. B. Choi, J. Y. Kim, H. J. Kim, Tetrahedron Lett. 1995, 36, 6253-6256; (u) S. Vettel, C. Lutz, P. Knochel, Synlett 1996, 731-733; (v) J. K. Gallos, L. C. Kyradjoglou, T. V. Koftis, Heterocycles 2001, 55, 781-784; (w) H.-Y. Lee, Y. Jung, H. Moon, Bull. Korean Chem. Soc. 2009, 30, 771-772).

(+)-Endo-brevicomin is a male produced component of the attractive pheromone system of Dendroctonus frontalis (southern pine beetle), a tree-killing insect found in southern North America and Central America (see R. M. Silverstein, R. G. Brownlee, T. E. Bellas, D. L. Wood, L. E. Browne, Science 1968, 159, 889-891). It was envisioned that AROCM of 10 with 4-penten-2-ol would set the relative and absolute stereochemistry in the synthesis of (+)-endo brevicomin.

An expedient three-step synthesis of (+)-endo brevicomin was accomplished featuring the AROCM of 10 with racemic 16 to afford 17 (91% Z) in 85% yield as an inconsequential mixture of diastereomers (Scheme 5). The mixture of epimeric alcohols was cleanly oxidized to the desired ketone by Dess-Martin periodinane in 88% yield. Z-18 was obtained in 95% ee, indicating high enantioselectivity in the AROCM reaction. Hydrogenation of Z-18 in acidic methanol resulted in concomitant reduction of the alkenes, hydrogenolysis of the benzyl groups and cyclization to form (+)-endo brevicomin in 67% yield in a one-pot transformation (The absolute configurations of the AROCM products in this study were assigned by analogy to 19 and 21).

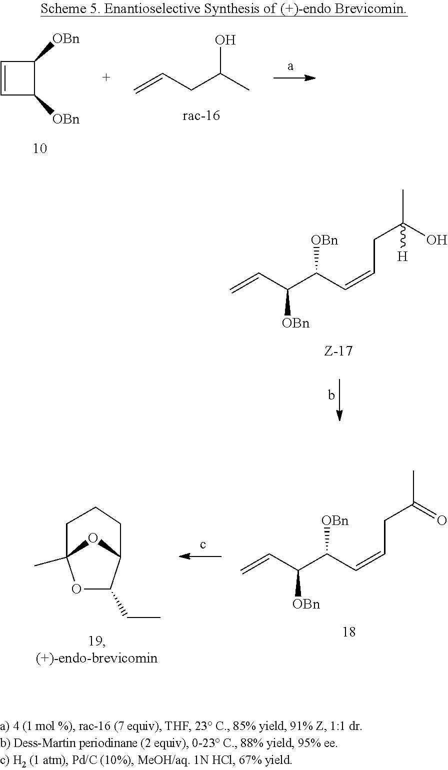

It was envisioned that the synthetic utility of the 1,5-dienes produced in the AROCM of cyclobutenes could be further underscored by chemoselective functionalization of the two alkenes. For example, the introduction of additional hydroxyl groups would enable the rapid synthesis of monosaccharides. In this fashion, a succinct and highly enantioselective synthesis of biologically relevant monosaccharides could function as a robust route to starting materials for complex polysaccharides.

The synthesis of ribose derivative 21 was carried out to demonstrate the conversion of AROCM products such as 15 into useful monosaccharides (Scheme 6). Dihydroxylation of Z-15f catalyzed by OsO₄ afforded a 66% yield of differentially protected pentanol 20 in 9:1 dr (see (a) J. K. Cha, W. J. Christ, Y. Kishi, Tetrahedron Lett. 1983, 24, 3943-3946; (b) W. J. Christ, J. K. Cha, Y. Kishi, Tetrahedron Lett. 1983, 24, 3947-3950). Ozonolysis of the remaining double bond afforded the differentially protected L-ribose lactol, which was isolated as methyl glycoside 21 in 47% yield over two steps (see (a) R. R. Schmidt, A. Gohl, Chem. Ber. 1979, 112, 1689-1704; (b) P. A. Wender, F. C. Bi, N. Buschmann, F. Gosselin, C. Kan, J.-M. Kee, H. Ohmura, Org. Lett. 2006, 8, 5373-5376). It is hypothesized that a broader collection of monosaccharides will be accessible from the AROCM products by the modification of this synthetic sequence.

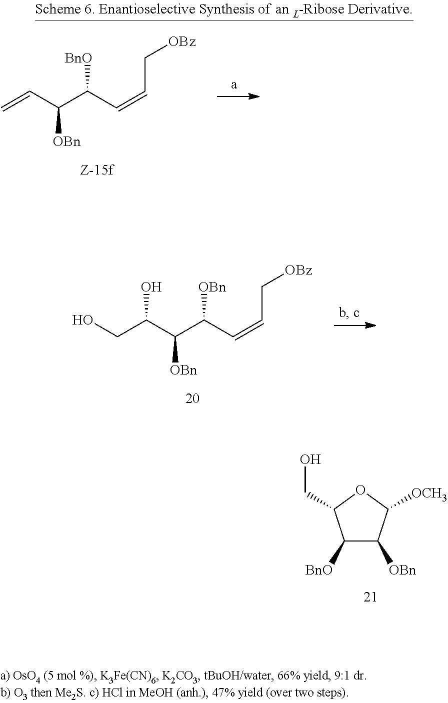

### Enatioenriched Catalysts

It was proposed that in addition to employing a catalyst with the large chelating adamantyl group (e.g. catalyst 4), further steric bulk could be installed by modification of the X-type ligand. In order to better understand how the X-type ligand affected the enantioselectivity, complexes 22a-h were prepared by ligand exchange from iodide 1. This reaction proceeded rapidly and afforded products of sufficient purity after concentration, re-dissolution in benzene, and filtration through a short plug of Celite (Scheme 7).

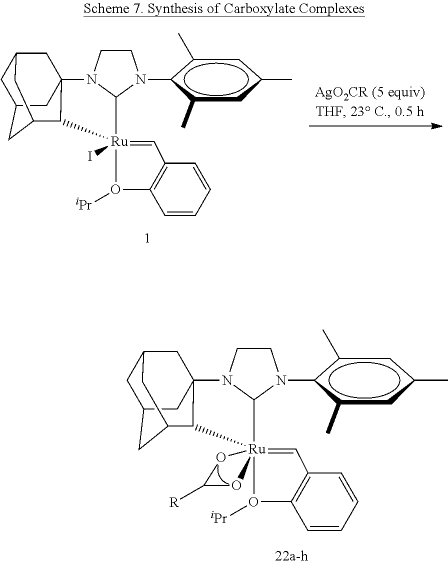

Complexes containing achiral carboxylates (22a-c) and enantiopure carboxylates (22d-h) were obtained (Scheme 8).

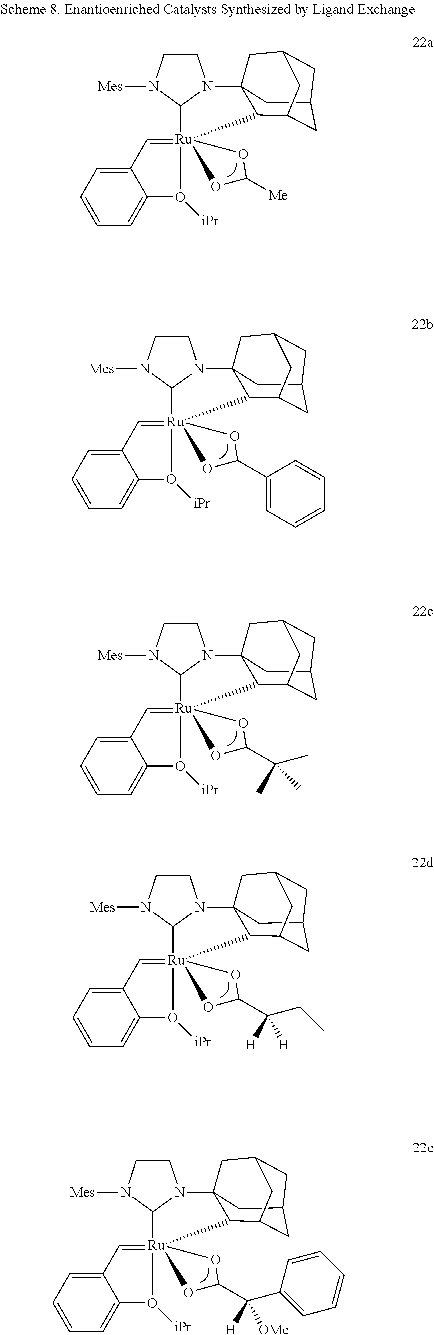

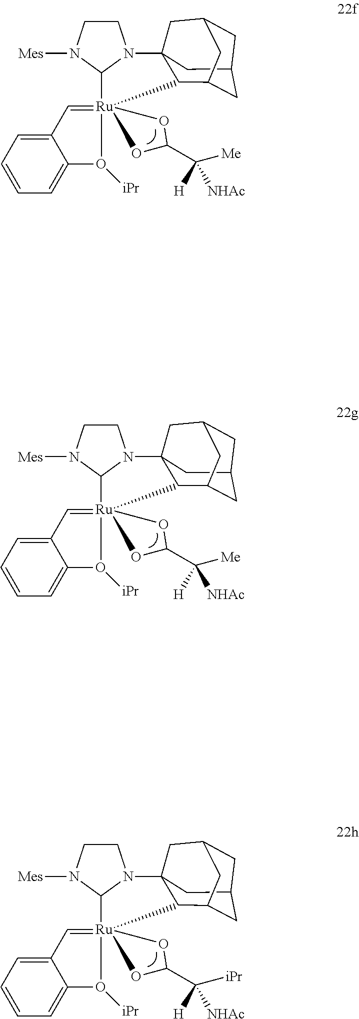

### Catalyzed Asymmetric Ring Opening/Cross Metathesis of Bicyclic Olefins and α-Olefins

Two of the novel catalysts depicted in Scheme 8 were employed in ring opening cross metathesis reactions (see Schemes x and x). While the O-methyl mandelate derived catalyst 22e afforded 57% yield of highly Z product, the enantioselectivity was modest (28%) (Scheme 9). The catalyst derived from L-N-acetyl alanine (221) afforded the ring opening cross product with >95% Z-selectivity and in 84% ee (Scheme 10).

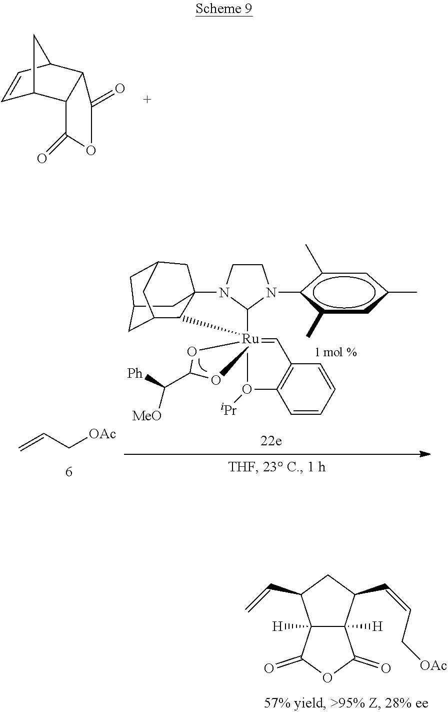

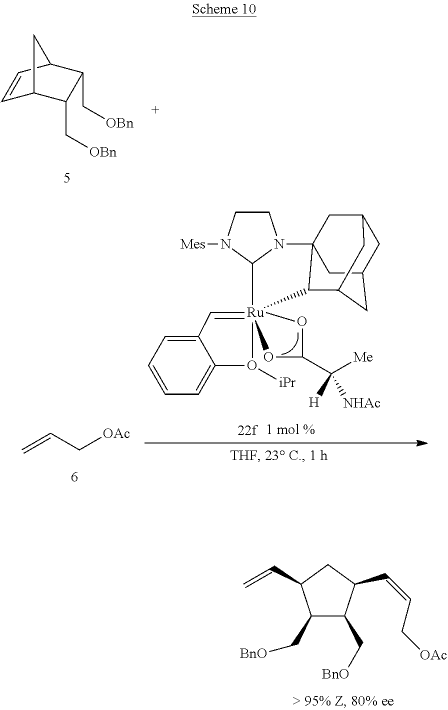

Nitrate 4 catalyzed the AROCM of benzonorbornadiene (23) with allyl acetate (6) in 55% yield, 76% Z-selectivity, while both Z and E isomers had >98% ee (see Scheme 11). AROCM of substrate 25, bearing the 7-syn benzyloxy substituent, afforded 26 as a mixture of isomers favoring the E product (18:85 Z/E ratio) in 94% and 93% ee (Z and E isomers respectively) (see Scheme 12).

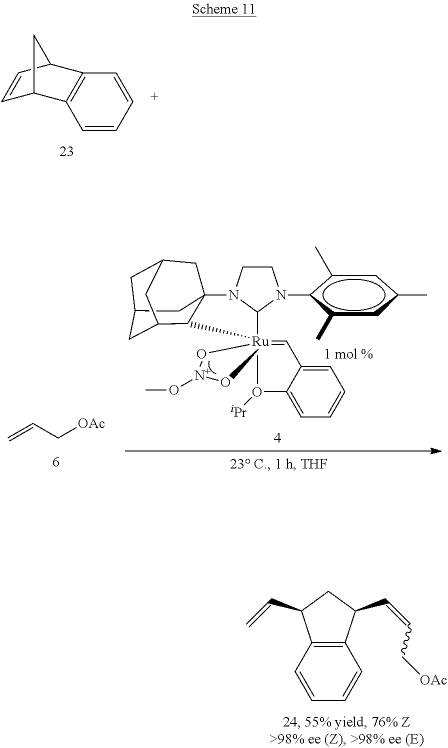

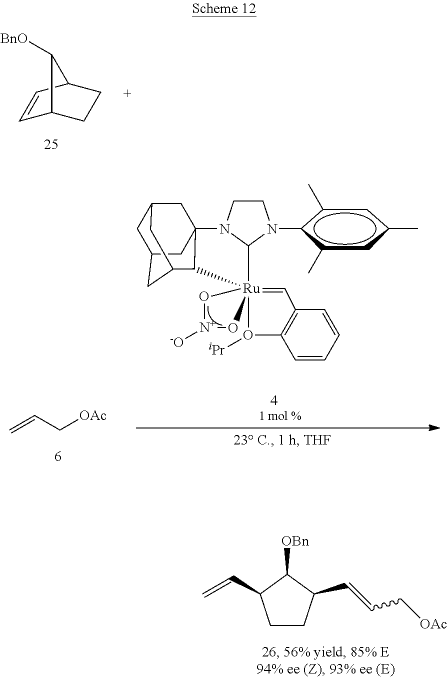

It is to be understood that while the invention has been described in conjunction with specific embodiments thereof, that the description above as well as the examples that follow are intended to illustrate and not limit the scope of the invention. Other aspects, advantages, and modifications within the scope of the invention will be apparent to those skilled in the art to which the invention pertains.

### EXPERIMENTAL

### General Information—Materials and Methods

In the following examples, efforts have been made to ensure accuracy with respect to numbers used (e.g., amounts, temperature, etc.) but some experimental error and deviation should be accounted for. Unless indicated otherwise, temperature is in degrees C. and pressure is at or near atmospheric. The examples are to be considered as not being limiting of the invention as described herein and are instead provided as representative examples of the catalyst compounds of the invention, the methods that may be used in their preparation, and the methods of using the inventive catalysts.

All reactions were carried out in dry glassware under an Argon atmosphere using standard Schlenk line techniques or in a Vacuum Atmospheres glovebox under nitrogen atmosphere. All solvents were purified by passage through solvent purification columns and further degassed with Argon (see Pangborn, A. B.; Giardello, M. A.; Grubbs, R. H.; Rosen, R. K.; Timmers, F. J., Organometallics 1996, 15, 1518-1520). NMR solvents for air-sensitive compounds were degassed by sparging with nitrogen and passed through a solvent purification column prior to use. Commercially available reagents were used as received unless otherwise noted. Substrates in the liquid state were degassed with Argon and passed through a plug of neutral alumina prior to use. Solid substrates were used after purification by silica gel column chromatography. Silica gel used for the purification of transition metal complexes was dried at 220° C. and 100 mTorr for 24 h prior to use.

Standard NMR spectroscopy experiments were conducted on a Varian INOVA 500 (¹H: 500 MHz, ¹³C: 125 MHz) spectrometer. Chemical shifts are referenced to the residual solvent peak (CDCl₃ or C₆D₆) multiplicity is reported as follows: (s: singlet, d: doublet, t: triplet: q: quartet, br: broad, m: multiplet). Spectra were analyzed and processed using MestReNova.

Gas chromatography data was obtained using an Agilent 6850 FID gas chromatograph equipped with an Agilent HP-5 5% phenyl methyl siloxane capillary column (J&W Scientific). GC instrument conditions: Inlet temperature—250° C.; Detector temperature—300° C.; Hydrogen flow—30 mL/min; Air flow—400 mL/min; Makeup flow—25 mL/min. GC method: 50° C. for 1 min, then temperature ramp (35° C./min) for 7 min to 300° C. followed by an isothermal period at 300° C. for 3 min.

Chiral gas chromatography was carried out on an Agilent 6850 FID gas chromatograph equipped with an Agilent GTA column. GC instrument conditions: Inlet temperature—180° C.; Detector temperature—250° C.; Hydrogen flow—32 mL/min; Air flow—400 mL/min; Makeup flow—30 mL/min. GC method: 80° C. for 12 min, isocratic.

High-resolution mass spectra (HRMS) data was obtained on a JEOL MSRoute mass spectrometer using FAB+, EI+, or MALDI-TOF methods.

Analytical SFC data was obtained on a Mettler SFC supercritical CO₂ analytical chromatography system equipped with Chiracel OD-H, OJ-H or Chirapak AD-H columns (4.6 mm×25 cm). Column temperature was maintained at 40° C. Preparative HPLC was conducted on an Agilent HPLC system equipped with Chiral Technologies Chiralpak AD-H column (21×250 mm) Optical rotations were measured on a Jasco P-2000 polarimeter using a 100 mm path-length cell at 589 nm.

### EXAMPLES

### Example 1

### Preparation of Silver Carboxylate 2

(S)-phenylmethoxy acetic acid (0.2 g, 1.2 mmol, 2 equiv.) was added to a stirring suspension of silver oxide (0.14 g, 0.6 mmol, 1 equiv.) in 5 mL deionized water shielded from light. The reaction was vigorously stirred for 3 h, at which time a light gray precipitate had formed. The mixture was filtered and washed with water, methanol, and hexanes. The resultant solid was dried under vacuum overnight while shielded from light to provide 0.264 g (0.971 mmol, 81% yield) of silver carboxylate 2. ¹H NMR (500 MHz, DMSO-d₆) δ 7.41-7.36 (m, 2H), 7.30-7.25 (m, 2H), 7.25-7.19 (m, 1H), 4.64 (s, 1H), 3.28 (s, 3H). ¹³C NMR (125 MHz, DMSO-d₆) δ 173.7, 139.8, 127.7, 127.2, 126.9, 84.9, 56.5. HRMS (MALDI-TOF) calculated for C₉H₉O₃ [M−Ag]⁻: 165.0552. found 165.0553.

### Example 2

### Preparation of Complex 3

Ruthenium iodide 1 (0.150 g, 0.215 mmol) and silver carboxylate 2 (0.117 g, 0.430 mmol, 2 equiv.) were added to a round bottom flask in a glovebox. THF (5 mL) was then added, and the suspension stirred for 1.5 h, at which time the color had changed from dark brown to purple. The mixture was concentrated and redissolved in benzene. The suspension was filtered through Celite and subsequently concentrated to afford a 1:1 mixture of carboxylates, 3 and 3′ (153 mg, 0.209 mmol, 97% yield). Pure carboxylate 3 (70.8 mg, 0.097 mmol, 90% of theoretical yield) was isolated by flash chromatography (2 cm×19 cm, 50% ether/pentane eluent) under an inert atmosphere. ¹H NMR (500 MHz, C₆D₆) δ 14.90 (s, 1H), 7.53-7.48 (m, 2H), 7.39 (dd, J=7.5, 1.7 Hz, 1H), 7.19-7.13 (m, 1H), 7.05-6.99 (m, 2H), 6.97-6.92 (m, 1H), 6.92-6.89 (m, 1H), 6.85-6.79 (m, 2H), 6.54 (d, J=8.4 Hz, 1H), 4.51 (m, 1H), 4.23 (s, 1H), 4.12 (s, 1H), 3.49 (m, 1H), 3.41-3.35 (m, 1H), 3.34 (s, 3H), 3.30-3.24 (m, 1H), 3.19 (m, 1H), 2.45 (s, 3H), 2.43 (s, 3H), 2.42-2.39 (m, 1H), 2.26 (s, 3H), 2.18-2.08 (m, 2H), 2.03 (m, 1H), 2.00-1.93 (m, 1H), 1.83 (m, 1H), 1.64 (br, 1H), 1.59-1.52 (m, 1H), 1.49 (m, 1H), 1.45-1.38 (m, 1H), 1.24 (d, J=6.5 Hz, 3H), 1.17-1.06 (m, 2H), 0.64-0.56 (m, 1H), 0.39 (d, J=6.2 Hz, 3H). ¹³C NMR (125 MHz, C₆D₆) δ 258.6, 214.9, 177.3, 154.4, 143.7, 139.2, 138.0, 137.9, 137.0, 136.3, 129.5, 129.5, 128.5, 128.0, 127.4, 125.5, 123.0, 122.9, 113.3, 84.9, 74.4, 69.2, 62.9, 56.5, 51.5, 43.3, 41.5, 40.6, 38.2, 38.0, 37.2, 33.6, 31.1, 29.9, 21.2, 21.2, 19.5, 18.9, 18.8. HRMS (FAB+) calculated for C₄₁H₄₉O₄RuN₂ [M−H⁻]: 735.2736. found 735.2757. The crystal structure of complex 3 is shown below in FIG. 2, (details are included in Tables 6-7).

TABLE 6
Crystal data and structural analysis details of 3.

| | | |
|---|---|---|
| Type of diffractometer | Bruker APEX-II CCD | |
| Wavelength | 0.71073 Å MoK | |
| Data collection temperature | 100 K | |
| Empirical formula | C₄₁H₅₀N₂O₄Ru | |
| Formula weight | 735.90 | |
| Crystal system | monoclinic | |
| Space group | P 1 21 1 (# 4) | |
| Theta range for | 2.76 to 42.71° | |
| 9040 reflections used | | |
| in lattice determination | | |
| Unit cell dimensions | a = 9.6582(6) Å | α = 90° |
| | b = 14.7827(9) Å | β = 105.749(3)° |
| | c = 12.9177(8) Å | γ = 90° |
| Volume | 1775.08(19) Å³ | |
| Z | 2 | |
| Density (calculated) | 1.377 g/cm³ | |
| F(000) | 772 | |
| Theta range for data | 1.6 to 43.7° | |
| collection | | |
| Completeness to | 99.9% | |
| theta = 25.000° | | |
| Index ranges | −18 ≦ h ≦ 18, −28 ≦ k ≦ 27, | |
| | −25 ≦ 1 ≦ 25 | |
| Reflections collected | 171955 | |
| Independent reflections | 26091 [Rᵢₙₜ = 0.0459] | |
| Reflections > 2σ(I) | 24002 | |
| Average σ(I)/(net I) | 0.0350 | |
| Absorption coefficient | 0.49 mm⁻¹ | |
| Absorption correction | Semi-empirical from | |
| | equivalents | |
| Max. and min. transmission | 1.0000 and 0.9117 | |
| Goodness-of-fit on F² | 1.24 | |
| Final R indices [I > 2σ(I), | R1 = 0.0252, wR2 = 0.0534 | |
| 24002 reflections] | | |
| R indices (all data) | R1 = 0.0303, wR2 = 0.0547 | |
| Primary solution method | dual | |
| Hydrogen placement | geom | |
| Refinement method | Full-matrix least-squares | |
| | on F² | |
| Data/restraints/parameters | 26091/1/439 | |
| Treatment of hydrogen | constr | |
| atoms | | |
| Type of weighting scheme | calc | |
| used | | |
| Weighting scheme used | | |
| Max shift/error | 0.001 | |
| Average shift/error | 0.000 | |
| Absolute structure parameter | −0.009(4) | |
| Extinction coefficient | n/a | |
| Largest diff. peak and hole | 1.15 and −0.44 e · Å⁻³ | |

TABLE 7
Atomic coordinates (×10⁴) and equivalent isotropic displacement
parameters (Å² × 10³) for 3. U(eq) is defined as
one third of the trace of the orthogonalized U^{ij} tensor.

| | x | y | z | U_{eq} |
|---|---|---|---|---|
| Ru(1) | 8019(1) | 7506(1) | 8590(1) | 8(1) |
| O(1) | 7144(1) | 6189(1) | 7675(1) | 12(1) |
| O(2) | 6468(1) | 7617(1) | 9731(1) | 14(1) |
| O(3) | 8396(1) | 6752(1) | 10126(1) | 12(1) |
| O(4) | 5955(1) | 7368(1) | 11626(1) | 19(1) |
| N(1) | 8574(1) | 9390(1) | 8447(1) | 13(1) |
| N(2) | 9586(1) | 9005(1) | 10111(1) | 15(1) |
| C(1) | 8834(1) | 8683(1) | 9138(1) | 11(1) |
| C(2) | 9015(1) | 10264(1) | 8967(1) | 18(1) |
| C(3) | 10043(2) | 9958(1) | 10029(2) | 20(1) |
| C(4) | 7660(1) | 9241(1) | 7359(1) | 11(1) |
| C(5) | 6838(1) | 8362(1) | 7423(1) | 10(1) |
| C(6) | 5957(1) | 8100(1) | 6286(1) | 13(1) |
| C(7) | 6896(1) | 8010(1) | 5506(1) | 16(1) |
| C(8) | 7659(1) | 8911(1) | 5443(1) | 16(1) |
| C(9) | 6537(1) | 9657(1) | 5043(1) | 16(1) |
| C(10) | 5611(1) | 9758(1) | 5832(1) | 15(1) |
| C(11) | 6569(1) | 10012(1) | 6956(1) | 15(1) |
| C(12) | 4835(1) | 8855(1) | 5873(1) | 16(1) |
| C(13) | 8594(1) | 9151(1) | 6567(1) | 15(1) |
| C(14) | 10092(1) | 8526(1) | 11100(1) | 14(1) |
| C(15) | 11297(1) | 7958(1) | 11276(1) | 15(1) |
| C(16) | 11805(1) | 7545(1) | 12283(1) | 18(1) |
| C(17) | 11158(2) | 7690(1) | 13107(1) | 21(1) |
| C(18) | 9965(2) | 8259(1) | 12906(1) | 20(1) |
| C(19) | 9417(1) | 8683(1) | 11915(1) | 17(1) |
| C(20) | 12040(1) | 7778(1) | 10415(1) | 21(1) |
| C(21) | 11727(2) | 7244(1) | 14189(1) | 31(1) |
| C(22) | 8095(2) | 9269(1) | 11741(1) | 25(1) |
| C(23) | 9469(1) | 7226(1) | 7959(1) | 12(1) |
| C(24) | 9291(1) | 6462(1) | 7223(1) | 12(1) |
| C(25) | 10239(1) | 6274(1) | 6600(1) | 17(1) |
| C(26) | 9944(2) | 5597(1) | 5824(1) | 21(1) |
| C(27) | 8694(2) | 5084(1) | 5676(1) | 21(1) |
| C(28) | 7745(1) | 5242(1) | 6296(1) | 16(1) |
| C(29) | 8034(1) | 5930(1) | 7054(1) | 12(1) |
| C(30) | 5784(1) | 5695(1) | 7524(1) | 14(1) |
| C(31) | 6063(1) | 4807(1) | 8131(1) | 22(1) |
| C(32) | 4742(1) | 6293(1) | 7899(1) | 18(1) |
| C(33) | 7310(1) | 7090(1) | 10359(1) | 11(1) |
| C(34) | 7073(1) | 6819(1) | 11439(1) | 13(1) |
| C(35) | 6738(1) | 5820(1) | 11442(1) | 13(1) |
| C(36) | 7738(1) | 5217(1) | 12033(1) | 25(1) |
| C(37) | 7458(2) | 4291(1) | 11982(2) | 34(1) |
| C(38) | 6163(2) | 3965(1) | 11337(1) | 28(1) |
| C(39) | 5138(2) | 4566(1) | 10771(1) | 27(1) |
| C(40) | 5419(1) | 5487(1) | 10819(1) | 22(1) |
| C(41) | 5945(2) | 7337(1) | 12722(1) | 25(1) |

### Example 3

### Preparation of Catalyst 4

To a solution of ruthenium carboxylate 3 (56.5 mg, 0.0769 mmol) in 5 mL THF was added para-toluenesulfonic acid monohydrate (14.6 mg, 0.0769 mmol, 1 equiv.) to instantly afford a green/blue solution. Sodium nitrate (32.7 mg, 0.384 mmol, 5 equiv.) was added and then methanol was added dropwise until the solution turned purple. The purple solution was allowed to stir for 15 min., at which time it was concentrated. The resultant crude mixture was redissolved in THF, filtered through Celite, and concentrated. Elution through a silica gel plug afforded pure nitrate 4 (21 mg, 0.033 mmol, 43% yield), which was spectroscopically identical to the previously reported complex (see Keitz, B. K.; Endo, K.; Patel, P. R.; Herbert, M. B.; Grubbs, R. H. J. Am. Chem. Soc. 2012, 134, 693).

### Substrates for Catalyzed AROCM of Bicyclic Olefins and α-Olefins

Substrates for AROCM were synthesized as previously reported in the literature: 5 (see Wang, L.; RajanBabu, T. V. J. Org. Chem. 2010, 75, 7636) and starting materials to generate 9a (see R. Alder Chem. Ber. 1955, 88, 407-416), 9b (see Takebayashi, S.; John, J. M.; Bergens, S. H. J. Am. Chem. Soc. 2010, 132, 12832-12834), 9d (see Tiede, S.; Berger, A.; Schlesiger, D.; Rost, D.; Lühl, A.; Blechert, S. Angew. Chem., Int. Ed. 2010, 49, 3972-3975), 9e (see Van Veldhuizen, J. J.; Garber, S. B.; Kingsbury, J. S.; Hoveyda, A. H. J. Am. Chem. Soc. 2002, 124, 4954-4955) were synthesized according to the provided references.

### General Procedure for Catalyzed AROCM of Bicyclic Olefins and α-Olefins

In a glovebox, norbornene 7 (33 mg, 0.1 mmol, 1 equiv) and allyl acetate (70 mg, 0.7 mmol, 7 equiv) were dissolved in 0.15 mL THF. To this solution was added 50 μL of a stock solution (0.02 M in THF) of catalyst 4. The reaction vial was capped and stirred for 1 h and then quenched with an excess of ethyl vinyl ether. The reaction mixture was concentrated and Z/E ratios were determined by 500 MHz ¹H NMR (products 7, 8a-e) or GC (products 9a-e). The crude was subjected to flash chromatography or preparative TLC to afford the desired AROCM product (7, 27.9 mg, 64% yield, 95:5 Z/E, 93% ee). Pure products were submitted to analytical SFC to determine ee.

### Example 4

Characterization data for AROCM product Benzyl ether 7, 64% yield, 95% Z. [α]_{D}²⁵+36.6° (c=1.39, CHCl₃); ¹H NMR (500 MHz, CDCl₃) δ 7.36-7.27 (m, 10H), 5.85 (ddd, J=17.1, 10.1, 8.3 Hz, 1H), 5.65 (t, J=10.7 Hz, 1H), 5.48 (m, 1H), 4.99 (ddd, J=17.1, 2.1, 1.1 Hz, 1H), 4.92 (ddd, J=10.2, 2.1, 0.8 Hz, 1H), 4.66 (ddd, J=12.7, 7.4, 1.3 Hz, 1H), 4.55 (ddd, J=12.6, 6.4, 1.4 Hz, 1H), 4.38 (dd, J=11.8, 2.3 Hz, 2H), 4.35 (d, J=11.8 Hz, 2H), 3.53-3.37 (m, 4H), 3.16-3.00 (m, 1H), 2.82-2.68 (m, 1H), 2.49-2.37 (m, 2H), 2.03 (s, 3H), 1.98 (dt, J=12.9, 8.2 Hz, 1H), 1.64-1.53 (m, 1H). ¹³C NMR (125 MHz, CDCl₃) δ 171.1, 140.5, 138.6, 138.6, 137.6, 128.4, 128.0, 127.9, 127.6, 123.2, 114.6, 73.4, 73.3, 68.8, 68.6, 60.6, 45.9, 45.8, 45.6, 38.8, 38.7, 21.2. HRMS (EI+) calculated for C₂₁H₂₇O₃ [M−OBn]: 327.1960. found 327.1966.

Separation conditions: OD-H, 5% IPA, 2.5 mL/min. 93% ee

### Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 23.467 | BV | 9.5111 | 1044.70398 | 30.53812 | 49.8296 |
| 2 | 25.004 | VB | 0.5167 | 1051.84814 | 28.89583 | 50.1704 |

| | | |
|---|---|---|
| Totals: | 2096.55212 | 59.43396 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 23.012 | BV | 0.3973 | 451.99329 | 14.12780 | 3.3052 |
| 2 | 24.210 | VB | 0.6641 | 1.32231e4 | 308.47403 | 96.6948 |

| | | |
|---|---|---|
| Totals: | 1.36751e4 | 322.60183 |

### Example 5

Characterization data for AROCM product Carbamate 8a, 41% yield, 95% Z. [α]_{D}²⁵+25.4° (c=0.50, CHCl₃); ¹H NMR (500 MHz, CDCl₃) δ 7.36-7.27 (m, 10H), 5.84 (ddd, J=17.1, 10.2, 8.2 Hz, 1H), 5.51 (t, J=10.5 Hz, 1H), 5.45-5.33 (m, 1H), 4.98 (ddd, J=17.1, 2.1, 1.1 Hz, 1H), 4.92 (ddd, J=10.2, 2.1, 0.8 Hz, 1H), 4.71 (s, 1H), 4.44-4.32 (m, 4H), 3.83-3.64 (m, 2H), 3.52-3.37 (m, 4H), 3.07 (m, 1H), 2.78-2.66 (m, 1H), 2.51-2.33 (m, 2H), 1.97 (dt, J=12.9, 8.2 Hz, 1H), 1.54-1.48 (m, 1H), 1.43 (s, 9H). ¹³C NMR (125 MHz, CDCl₃) δ 155.9, 140.3, 138.6, 138.5, 135.9, 128.4, 128.4, 128.4, 128.0, 127.9, 127.7, 127.6, 127.6, 125.6, 114.6, 73.3, 73.2, 68.8, 68.6, 46.0, 45.8, 45.8, 38.6, 38.2, 37.6, 28.6. HRMS (EI+) calculated for C₃₁H₄₁O₄N [M+]: 491.3036. found 491.3038.

Separation conditions: OD-H, 10% IPA, 2.5 mL/min. 94% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 16.314 | BB | 0.4076 | 2501.07446 | 93.63906 | 51.1753 |
| 2 | 19.228 | BBA | 0.4768 | 2386.19580 | 76.72945 | 48.8247 |

| | | |
|---|---|---|
| Totals: | 4887.27026 | 170.36851 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 16.842 | BB | 0.3219 | 126.93304 | 4.77848 | 2.7575 |
| 2 | 19.674 | BB | 0.4971 | 4476.19385 | 141.46957 | 97.2425 |

| | | |
|---|---|---|
| Totals: | 4603.12689 | 146.24805 |

### Example 6

Characterization data for AROCM product Ester 8b, 65% yield, 95% Z, ee was determined on derivative S1. [α]_{D}²⁵+31.8° (c=1.53, CHCl₃); ¹H NMR (500 MHz, CDCl₃) δ 7.35-7.26 (m, 10H), 5.86 (ddd, J=17.1, 10.1, 8.4 Hz, 1H), 5.46-5.37 (m, 1H), 5.35-5.26 (m, 1H), 4.97 (ddd, J=17.1, 2.2, 1.1 Hz, 1H), 4.90 (ddd, J=10.1, 2.2, 0.8 Hz, 1H), 4.44-4.32 (m, 4H), 4.12 (q, J=7.1 Hz, 2H), 3.52-3.38 (m, 4H), 3.12-2.99 (m, 1H), 2.79-2.68 (m, 1H), 2.55-2.24 (m, 6H), 1.96 (dt, J=12.8, 8.2 Hz, 1H), 1.58-1.48 (m, 1H), 1.25 (t, J=7.1 Hz, 3H). ¹³C NMR (125 MHz, CDCl₃) δ 173.3, 140.9, 138.7, 138.7, 133.5, 128.4, 128.4, 128.0, 127.9, 127.6, 127.6, 127.5, 114.3, 73.32, 73.30, 68.9, 68.8, 60.4, 45.8, 45.7, 45.6, 38.8, 38.6, 34.7, 23.1, 14.4. HRMS (EI+) calculated for C₃₀H₃₈O₄ [M+]: 462.2770. found 462.2758.

### Example 7

Characterization data for AROCM product Alcohol S1,

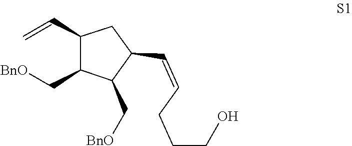 [SMILES: C=C[C@H]1C[C@@H](/C=C\CCCO)[C@@H](COCc2ccccc2)[C@H]1COCc1ccccc1]

Ester 8b was treated with excess DIBAL at 23° C. for 2 h to afford 76% yield of alcohol S1 after workup and silica gel chromatography.

[α]_{D}²⁵+31.3° (c=1.05, CHCl₃); ¹H NMR (500 MHz, CDCl₃) δ 7.37-7.27 (m, 10H), 5.86 (ddd, J=17.9, 10.0, 8.3 Hz, 1H), 5.46-5.29 (m, 2H), 4.98 (dd, J=17.2, 1.9 Hz, 1H), 4.91 (dd, J=10.1, 2.0 Hz, 1H), 4.48-4.31 (m, 4H), 3.60 (t, J=6.4 Hz, 2H), 3.46 (m, 4H), 3.06 (m, 1H), 2.74 (m, 1H), 2.43 (m, 2H), 2.12 (q, J=7.3 Hz, 2H), 1.97 (dt, J=12.7, 8.2 Hz, 1H), 1.68-1.47 (m, 3H), 1.44 (s, 1H). ¹³C NMR (125 MHz, CDCl₃) δ 140.8, 138.7, 138.6, 132.8, 129.1, 128.4, 128.4, 128.3, 128.0, 127.9, 127.6, 114.3, 73.32, 73.31, 73.28, 69.0, 68.8, 62.6, 45.7, 45.6, 38.8, 38.5, 32.7, 23.8. HRMS (FAB+) calculated for C₂₈H₃₇O₃ [M+H]: 421.2743. found 421.2746.

Separation conditions: OD-H, 15% IPA, 2.5 mL/min. 91% ee

Racemate:

Signal 1: DAD1 A, Sig = 210, 8 Ref = 360,100

| Peak | RetTime | Type | Width | Area | Height | Area |
|---|---|---|---|---|---|---|
| # | (min) | | (min) | (mAU*s) | (mAU) | % |
| 1 | 9.363 | BB | 0.2835 | 837.48871 | 44.92727 | 49.7025 |
| 2 | 10.229 | BB | 0.3081 | 847.51575 | 41.18006 | 50.2975 |

| | | |
|---|---|---|
| Totals: | 1685.00446 | 86.10733 |

Enantioenriched

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 9.451 | BV | 0.3229 | 1.03224e4 | 491.82324 | 95.3861 |
| 2 | 10.493 | VB | 0.3077 | 499.30240 | 21.13039 | 4.6139 |

| | | |
|---|---|---|
| Totals: | 1.08217e4 | 512.95363 |

### Example 8

Characterization data for AROCM product Benzyl ether 8c, 51% yield, 95% Z. [α]_{D}²⁵+32.9° (c=1.23, CHCl₃); ¹H NMR (500 MHz, CDCl₃) δ 7.42-7.22 (m, 10H), 7.08 (d, J=8.2 Hz, 2H), 6.82 (d, J=8.7 Hz, 2H), 5.89 (m, 1H), 5.50 (dd, J=6.5, 2.5 Hz, 2H), 5.00 (d, J=17.5 Hz, 1H), 4.92 (d, J=10.1 Hz, 1H), 4.48-4.33 (m, 4H), 3.78 (s, 3H), 3.60-3.44 (m, 4H), 3.39 (dd, J=15.6, 5.8 Hz, 1H), 3.29 (dd, J=15.6, 5.3 Hz, 1H), 3.17 (m, 1H), 2.77 (m, 1H), 2.58-2.37 (m, 2H), 2.02 (dt, J=12.7, 8.1 Hz, 1H), 1.67-1.55 (m, 1H). ¹³C NMR (125 MHz, CDCl₃) δ 157.9, 140.9, 138.7, 138.69, 133.3, 132.7, 129.3, 128.6, 128.4, 128.4, 128.0, 128.0, 127.9, 127.6, 127.57, 114.3, 113.9, 73.35, 73.33, 69.0, 68.9, 55.4, 45.8, 45.76, 45.74, 45.7, 38.9, 38.7, 32.8. HRMS (FAB+) calculated for C₃₃H₃₉O₃ [M+H]: 483.2899. found 483.2878.

Separation conditions: AD-H, 20% IPA, 2.5 mL/min. 81% ee

Racemate

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 5.678 | BV | 0.1849 | 4131.27295 | 328.10538 | 49.8705 |
| 2 | 6.289 | VB | 0.2028 | 4152.73145 | 297.64392 | 50.1295 |

| | | |
|---|---|---|
| Totals: | 8284.00439 | 625.74930 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 5.658 | VV | 0.1862 | 986.96069 | 78.75704 | 9.4299 |
| 2 | 6.263 | VB | 0.2132 | 9479.31836 | 661.58344 | 90.5701 |

| | | |
|---|---|---|
| Totals: | 1.04663e4 | 740.34048 |

### Example 9

Characterization data for AROCM product Boronic ester 8d, 48% yield, 95% Z. [α]_{D}²⁵+12.8° (c=0.85, CHCl₃); ¹H NMR (500 MHz, CDCl₃) δ 7.35-7.26 (m, 10H), 5.88 (ddd, J=17.1, 10.1, 8.5 Hz, 1H), 5.54-5.43 (m, 1H), 5.41-5.32 (m, 1H), 4.97 (ddd, J=17.1, 2.2, 1.1 Hz, 1H), 4.89 (ddd, J=10.1, 2.2, 0.8 Hz, 1H), 4.39 (dd, J=11.8, 3.9 Hz, 2H), 4.34 (dd, J=11.8, 3.8 Hz, 2H), 3.54-3.41 (m, 4H), 3.02 (m, 1H), 2.81-2.65 (m, 1H), 2.51-2.33 (m, 2H), 1.97 (dt, J=12.9, 8.2 Hz, 1H), 1.76-1.59 (m, 2H), 1.57-1.49 (m, 1H), 1.23 (s, 12H). ¹³C NMR (125 MHz, CDCl₃) δ 141.2, 138.8, 132.0, 128.4, 127.9, 127.51, 127.50, 124.3, 114.0, 83.3, 73.3, 73.26, 69.1, 69.0, 45.70, 45.68, 45.3, 38.7, 38.6, 24.92, 24.89. HRMS (EI+) calculated for C₃₂H₄₃O₄B [M+]: 502.3254. found 502.3252.

Separation conditions: OD-H, 5% IPA, 2.5 mL/min. 75% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 21.313 | BB | 0.4859 | 1988.34473 | 61.38202 | 48.5113 |
| 2 | 223.201 | BB | 0.5323 | 2110.38379 | 58.03347 | 51.4887 |

| | | |
|---|---|---|
| Totals: | 4098.72852 | 119.41549 |

Enantoenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 21.095 | BB | 0.4365 | 310.99133 | 9.53655 | 12.3902 |
| 2 | 22.889 | BB | 0.5353 | 2198.98804 | 62.99510 | 87.6098 |

| | | |
|---|---|---|
| Totals: | 2509.97937 | 72.53165 |

### Example 10

Characterization data for AROCM product Benzyl ether 8e, 62% yield, 95% Z. [α]_{D}²⁵+28.7° (c=1.3, CHCl₃); ¹H NMR (500 MHz, CDCl₃) δ 7.35-7.25 (m, 10H), 5.87 (ddd, J=17.1, 10.1, 8.4 Hz, 1H), 5.39-5.28 (m, 2H), 4.97 (ddd, J=17.2, 2.2, 1.1 Hz, 1H), 4.89 (ddd, J=10.2, 2.2, 0.8 Hz, 1H), 4.42-4.34 (m, 4H), 3.55-3.39 (m, 4H), 3.10-2.93 (m, 1H), 2.74 (m, 1H), 2.46 (m, 1H), 2.38 (m, 1H), 2.12-1.84 (m, 2H), 1.52 (m, 1H), 1.35-1.24 (m, 5H), 0.94-0.81 (m, 3H). ¹³C NMR (125 MHz, CDCl₃) δ 141.1, 138.8, 131.9, 130.2, 128.40, 128.38, 128.0, 127.95, 127.94, 127.6, 127.5, 114.1, 73.3, 73.3, 69.1, 68.9, 45.7, 45.6, 38.9, 38.7, 32.2, 27.3, 22.5, 14.2. HRMS (EI+) calculated for C₂₉H₃₈O₂ [M+]: 418.2872. found 418.2856.

Separation conditions: OJ-H, 5% IPA, 2.5 mL/min. 89% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 10.200 | MM | 0.4618 | 2528.33081 | 91.24889 | 51.9762 |
| 2 | 12.974 | MM | 0.5422 | 2336.07202 | 71.80923 | 48.0238 |

| | | |
|---|---|---|
| Totals: | 4864.40283 | 163.05812 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 10.177 | VV | 0.3707 | 1.44529e4 | 588.22974 | 94.6063 |
| 2 | 13.210 | VV | 0.3531 | 823.99506 | 30.31367 | 5.3937 |

| | | |
|---|---|---|
| Totals: | 1.52769e4 | 618.54341 |

### Example 11

Characterization data for AROCM product Triacetate 9a, 45% yield, 97% Z. [α]_{D}²⁵+23.9° (c=0.58, CHCl₃); ¹H NMR (500 MHz, CDCl₃) δ 5.80 (ddd, J=17.0, 10.3, 8.0 Hz, 1H), 5.62-5.52 (m, 2H), 5.04 (ddd, J=11.9, 1.8, 1.1 Hz, 1H), 5.02 (ddd, J=5.1, 1.8, 1.1 Hz, 1H), 4.71-4.65 (m, 1H), 4.60-4.55 (m, 1H), 4.14-3.97 (m, 4H), 3.15 (m, 1H), 2.87-2.76 (m, 1H), 2.51 (m, 2H), 2.12 (dt, J=13.4, 8.3 Hz, 1H), 2.06 (s, 3H), 2.03 (s, 3H), 2.03 (s, 3H), 1.49 (m, 1H). ¹³C NMR (125 MHz, CDCl₃) δ 171.1, 170.97, 170.94, 138.8, 136.0, 124.8, 115.9, 62.8, 62.7, 60.3, 45.2, 44.6, 44.3, 38.2, 37.7, 21.2, 21.1.

HRMS (EI) calculated for C₁₈H₂₆O₆ [M+]: 338.1729. found 338.1737.

Separation conditions: OD-H, 3% IPA, 2.5 mL/min. 82% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 6.037 | BB | 0.1736 | 201.35269 | 18.13161 | 45.1499 |
| 2 | 7.186 | BB | 0.1999 | 244.61229 | 19.04047 | 54.8501 |

| | | |
|---|---|---|
| Totals: | 445.96498 | 37.17208 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 6.110 | BB | 0.1233 | 39.40160 | 4.28349 | 9.1737 |
| 2 | 7.154 | BB | 0.1978 | 390.10281 | 29.59711 | 90.8263 |

| | | |
|---|---|---|
| Totals: | 429.50441 | 33.88060 |

### Example 12

Characterization data for AROCM product Imide 9b. The standard conditions were modified to employ 3 mol % of 4 for 5 h. 63% yield, 94% Z. [α]_{D}²⁵+14.4° (c=0.28, CHCl₃); ¹H NMR (500 MHz, CDCl₃) δ 7.48-7.43 (m, 2H), 7.40-7.35 (m, 1H), 7.26-7.23 (m, 2H), 6.06 (ddd, J=17.4, 10.0, 7.3 Hz, 1H), 5.83-5.76 (m, 1H), 5.76-5.69 (m, 1H), 5.17 (m, 1H), 5.15-5.13 (m, 1H), 4.74-4.70 (m, 1H), 4.70-4.66 (m, 1H), 3.46-3.30 (m, 3H), 3.12-3.01 (m, 1H), 2.07 (s, 3H), 2.04-1.97 (m, 1H), 1.48 (m, 1H). ¹³C NMR (125 MHz, CDCl₃) δ 175.6, 175.5, 141.1, 136.1, 132.7, 131.9, 129.2, 128.7, 126.5, 116.3, 60.1, 49.3, 48.9, 46.6, 40.4, 37.3, 21.2. HRMS (EI) calculated for C₂₀H₂₁O₄N [M+]: 339.1471. found 339.1473.

Separation conditions: AD-H, 10% IPA, 2.5 mL/min. 60% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.263 | BB | 0.4040 | 1182.51379 | 44.22358 | 50.6270 |
| 2 | 12.608 | BB | 0.4544 | 1153.22241 | 38.40098 | 49.3730 |

| | | |
|---|---|---|
| Totals: | 2335.73621 | 82.62455 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.366 | BV | 0.3278 | 2275.38013 | 99.12678 | 79.8303 |
| 2 | 12.792 | BB | 0.3554 | 574.89026 | 22.54995 | 20.1697 |

### Example 13

Characterization data for AROCM product Anhydride 9c, 58% yield, 98% Z. The ee of anhydride 9c produced by chiral catalyst 4 was measured on derivative S2. [α]_{D}²⁵+1.74° (c=0.73, CHCl₃); ¹H NMR (500 MHz, CDCl₃) δ 5.93 (ddd, J=17.0, 10.4, 7.4 Hz, 1H), 5.76 (ddd, J=10.9, 7.1, 1H), 5.67 (ddd, J=11.1, 9.9, 1H), 5.20 (ddd, J=6.3, 1.3 Hz, 1H), 5.17 (ddd, J=12.8, 1.3 Hz, 1H), 4.68 (ddd, J=12.8, 6.9, 1.3 Hz, 1H), 4.63 (ddd, J=12.8, 7.2, 1.2 Hz, 1H), 3.55-3.46 (m, 2H), 3.42-3.33 (m, 1H), 3.11-2.97 (m, 1H), 2.06 (s, 3H), 2.06-2.00 (m, 1H), 1.41 (m, 1H). ¹³C NMR (125 MHz, CDCl₃) δ 171.0, 170.6, 170.5, 134.7, 131.4, 126.7, 117.6, 59.8, 49.69, 49.68, 49.51, 49.50, 47.0, 40.7, 37.6, 21.1. HRMS (EI) calculated for C₁₄H₁₆O₅ [M+]: 264.0998. found 264.0989.

### Example 14

Characterization data for AROCM product Imide S2. Anhydride 9c was treated with p-bromo aniline (xylenes, reflux, 20 h, 60% yield) to afford the imide S2.

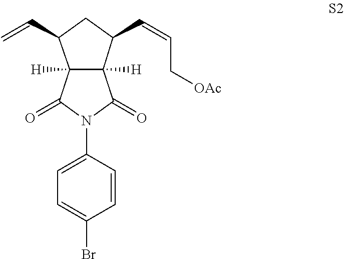 [SMILES: C=C[C@H]1C[C@@H](/C=C\COC(C)=O)[C@H]2C(=O)N(c3ccc(Br)cc3)C(=O)[C@H]21]

[α]_{D}²⁵+12.5° (c=0.28, CHCl₃); ¹H NMR (500 MHz, CDCl₃) δ 7.63-7.53 (m, 2H), 7.19-7.09 (m, 2H), 6.12-5.96 (m, 1H), 5.79-5.68 (m, 2H), 5.17 (m, 1H), 5.14 (ddd, J=5.9, 1.4 Hz, 1H), 4.73-4.69 (m, 1H), 4.69 (m, 1H), 3.47-3.29 (m, 3H), 3.13-3.00 (m, 1H), 2.07 (s, 3H), 2.05-1.96 (m, 1H), 1.50-1.37 (m, 1H). ¹³C NMR (125 MHz, CDCl₃) δ 175.3, 175.1, 171.0, 135.9, 132.5, 132.4, 130.9, 128.0, 125.8, 122.5, 116.4, 60.1, 49.3, 48.9, 46.5, 40.4, 37.2, 21.2. HRMS (FAB+) calculated for C₂₀H₂₁O₄N⁸¹Br [M+H]: 420.0633. found 420.0624.

Separation conditions: AD-H, 10% IPA, 2.5 mL/min, 75% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 20.134 | VV | 0.6044 | 1900.42920 | 38.59682 | 48.6923 |
| 2 | 21.922 | CB | 0.5698 | 2002.50818 | 42.03310 | 51.3077 |

| | | |
|---|---|---|
| Totals: | 3902.93738 | 80.62992 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 20.018 | BB | 0.4571 | 208.39857 | 5.43821 | 12.4835 |
| 2 | 21.777 | BB | 0.4991 | 1460.98792 | 36.02553 | 87.5165 |

| | | |
|---|---|---|
| Totals: | 1669.38649 | 41.46374 |

### Example 15

Characterization data for AROCM product Imide ent-S3.

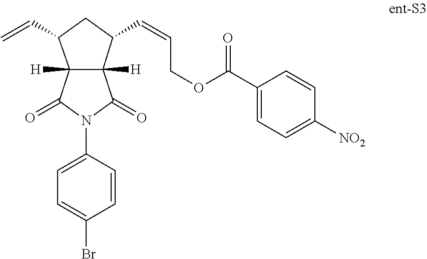 [SMILES: C=C[C@@H]1C[C@H](/C=C\COC(=O)c2ccc([N+](=O)[O-])cc2)[C@@H]2C(=O)N(c3ccc(Br)cc3)C(=O)[C@@H]21]

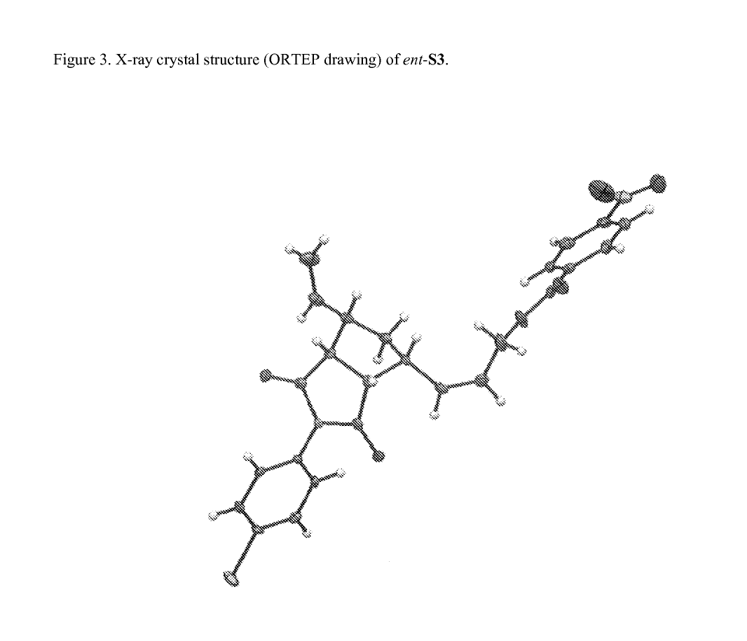

In order to determine the absolute configuration of AROCM products, imides S2 (major product) and ent-S2 (minor product) were separated by preparative chiral HPLC to afford pure samples (>99% e.e.) of each enantiomer. The acetate of imide ent-S2 was removed and the resultant alcohol was acylated with p-nitro benzoyl chloride to give imide ent-S3. [α]_{D}²⁵-34° (c=0.09, CHCl₃); ¹H NMR (500 MHz, CDCl₃) δ 8.33-8.27 (m, 2H), 8.25-8.18 (m, 2H), 7.61-7.55 (m, 2H), 7.19-7.13 (m, 2H), 6.14-5.97 (m, 1H), 5.90-5.82 (m, 2H), 5.19 (m, 1H), 5.16 (m, 1H), 5.02-4.99 (m, 2H), 3.44 (m, 3H), 3.09 (m, 1H), 2.11-2.00 (m, 2H), 1.50 (m, 1H). ¹³C NMR (125 MHz, CDCl₃) δ 175.2, 175.1, 175.0, 135.85, 135.83, 133.5, 132.4, 130.9, 128.9, 128.8, 128.0, 125.1, 123.7, 122.6, 116.5, 61.4, 49.4, 48.9, 46.6, 40.5, 37.3. The crystal structure of ent-S3 is shown below in FIG. 3, (details are included in Tables 8-9).

TABLE 8
Crystal Data and Structure Analysis Details for ent-S3.

| | | |
|---|---|---|
| Type of diffractometer | Bruker APEX-II CCD | |
| Wavelength | 0.71073 Å MoK | |
| Data collection temperature | 100 K | |
| Empirical formula | C₂₅H₂₁BrN₂O₆ | |
| Formula weight | 525.35 | |
| Crystal system | orthorhombic | |
| Space group | P 21 21 21 (# 19) | |
| Theta range for 7125 reflections | 2.58 to 24.81° | |
| used in lattice determination | | |
| Unit cell dimensions | a = 6.8772(3) Å | α = 90° |
| | b = 15.7930(10) Å | β = 90° |
| | c = 20.9413(12) Å | γ = 90° |
| Volume | 2274.5(2) Å³ | |
| Z | 4 | |
| Density (calculated) | 1.534 g/cm3 | |
| F(000) | 1072 | |
| Theta range for data collection | 1.9 to 29.8° | |
| Completeness to theta = 25.000° | 99.9% | |
| Index ranges | −9 ≦ h ≦ 9, −20 ≦ k ≦ 21, | |
| | −27 ≦ 1 ≦ 28 | |
| Reflections collected | 34241 | |
| Independent reflections | 5864 [Rᵢₙₜ = 0.0777] | |
| Reflections > 2σ(I) | 4359 | |
| Average σ(I)/(net I) | 0.0925 | |
| Absorption coefficient | 1.85 mm-1 | |
| Absorption correction | Semi-empirical from | |
| | equivalents | |
| Max. and min. transmission | 1.0000 and 0.8351 | |
| Goodness-of-fit on F2 | 1.04 | |
| Final R indices [I > 2σ(I), 4359 | R1 = 0.0434, | |
| reflections] | wR2 = 0.0558 | |
| R indices (all data) | R1 = 0.0789, | |
| | wR2 = 0.0614 | |
| Primary solution method | dual | |
| Hydrogen placement | geom | |
| Refinement method | Full-matrix least-squares | |
| | on F2 | |
| Data/restraints/parameters | 5864/0/307 | |
| Treatment of hydrogen atoms | constr | |
| Type of weighting scheme used | calc | |
| Weighting scheme used | | |
| Max shift/error | 0.000 | |
| Average shift/error | 0.000 | |
| Absolute structure parameter | 0.003(5) | |
| Extinction coefficient | n/a | |
| Largest diff. peak and hole | 0.67 and −0.47 e · Å-3 | |

TABLE 9
Atomic coordinates (×10⁴) and equivalent isotropic
displacement parameters (Å² × 10³) for ent-S3. U(eq) is
defined as one third of the trace of the orthogonalized U^{ij} tensor.

| | x | y | z | Ueq |
|---|---|---|---|---|
| Br(1) | 5721(1) | 4492(1) | −1710(1) | 27(1) |
| O(1) | 7832(3) | 7442(2) | 500(1) | 17(1) |
| O(2) | 13037(3) | 5688(2) | 266(1) | 19(1) |
| O(3) | 8803(3) | 8294(2) | 3469(1) | 25(1) |
| O(4) | 9757(3) | 9519(2) | 3918(1) | 26(1) |
| O(5) | 8568(4) | 7556(2) | 6851(1) | 38(1) |
| O(6) | 9132(5) | 6354(2) | 6395(1) | 47(1) |
| N(1) | 10188(3) | 6447(2) | 308(1) | 10(1) |
| N(2) | 8876(4) | 7120(2) | 6378(2) | 25(1) |
| C(1) | 9451(5) | 7168(2) | 596(2) | 13(1) |
| C(2) | 10992(5) | 7535(2) | 1028(2) | 12(1) |
| C(3) | 10491(5) | 7511(2) | 1751(2) | 15(1) |
| C(4) | 11203(4) | 6635(2) | 1955(2) | 16(1) |
| C(5) | 13191(4) | 6577(2) | 1631(2) | 15(1) |
| C(6) | 12773(4) | 6936(2) | 953(2) | 12(1) |
| C(7) | 12122(5) | 6280(2) | 478(2) | 14(1) |
| C(8) | 9172(5) | 5975(2) | −170(1) | 10(1) |
| C(9) | 9911(4) | 5922(2) | −784(2) | 12(1) |
| C(10) | 8912(4) | 5470(2) | −1245(2) | 16(1) |
| C(11) | 7170(5) | 5089(2) | −1084(2) | 14(1) |
| C(12) | 6416(5) | 5143(2) | −474(2) | 14(1) |
| C(13) | 7435(4) | 5587(2) | −11(1) | 11(1) |
| C(14) | 8404(5) | 7685(3) | 1925(2) | 20(1) |
| C(15) | 7773(5) | 8174(3) | 2392(2) | 24(1) |
| C(16) | 8962(6) | 8693(2) | 2838(2) | 26(1) |
| C(17) | 9259(6) | 8795(2) | 3967(2) | 18(1) |
| C(18) | 9085(5) | 8327(2) | 4588(2) | 16(1) |
| C(19) | 9040(5) | 8808(2) | 5143(2) | 17(1) |
| C(20) | 8959(5) | 8416(2) | 5733(2) | 18(1) |
| C(21) | 8944(5) | 7548(2) | 5749(2) | 17(1) |
| C(22) | 8985(5) | 7049(2) | 5209(2) | 21(1) |
| C(23) | 9046(5) | 7449(2) | 4620(2) | 18(1) |
| C(24) | 14163(5) | 5731(2) | 1661(2) | 20(1) |
| C(25) | 15742(6) | 5574(3) | 1992(2) | 34(1) |

### Example 16

Characterization data for AROCM product Anhydride 9d, 65% yield, 96% Z. [α]_{D}²⁵+1.96° (c=0.57, CHCl₃); ¹H NMR (500 MHz, CDCl₃) δ 5.89-5.76 (m, 2H), 5.64 (t, J=10.8 Hz, 1H), 5.20 (d, J=10.2 Hz, 1H), 5.16 (d, J=16.9 Hz, 1H), 4.70 (dd, J=12.7, 7.6 Hz, 1H), 4.59 (dd, J=12.7, 6.6 Hz, 1H), 2.96-2.83 (m, 1H), 2.52 (m, 1H), 2.07 (s, 3H), 1.94 (m, 1H), 1.40 (m, 1H), 1.33 (s, 3H), 1.28 (s, 3H). ¹³C NMR (125 MHz, CDCl₃) δ 173.9, 173.7, 171.0, 134.6, 131.4, 127.3, 118.3, 59.8, 57.8, 57.2, 55.3, 48.2, 37.0, 21.1, 18.3, 18.3. HRMS (FAB+) calculated for C₁₆H₂₁O₅ [M+H]: 293.1389. found 293.1394.

Separation conditions: AD-H, 3% IPA, 2.5 mL/min, 95% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 4.099 | BV | 0.2706 | 727.27740 | 33.32114 | 49.5823 |
| 2 | 4.736 | VB | 0.2826 | 739.53021 | 32.60941 | 50.4177 |

| | | |
|---|---|---|
| Totals: | 1466.80762 | 65.93055 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 4.135 | BB | 0.2324 | 20.50533 | 1.26954 | 2.4090 |
| 2 | 4.727 | VB | 0.2981 | 830.67578 | 38.13840 | 97.5910 |

| | | |
|---|---|---|
| Totals: | 851.18111 | 39.40794 |

### Example 17

Characterization data for AROCM product Aryl ether 9e. Isolated as a 7:3 mixture of Z/E olefin isomers, 40% yield. ¹H NMR (500 MHz, CDCl₃) δ 7.49 (m, 2H), 6.96-6.89 (m, 3H), 5.84 (ddd, J=17.1, 10.3, 7.8 Hz, 2H), 5.82-5.74 (m, 1H), 5.68-5.59 (m, 1H), 5.59-5.51 (m, 1H), 5.09 (m, 1H), 5.04 (m, 1H), 4.69 (m, 1H), 4.52-4.44 (m, 2H), 4.29 (m, 1H), 4.25 (m, 1H), 3.09 (m, 1H), 2.78 (m, 2H), 2.05 (m, 1H), 2.04 (s, 3H), 1.99 (s, 3H), 1.73-1.60 (m, 2H), 1.61-1.50 (m, 1H). ¹³C NMR (125 MHz, CDCl₃) δ 171.0, 170.9, 161.2, 139.5, 139.4, 136.7, 136.3, 126.8, 125.0, 116.2, 116.1, 115.7, 115.6, 89.2, 88.7, 64.9, 60.5, 49.9, 49.9, 49.0, 44.7, 30.2, 29.3, 29.2, 29.16, 21.12, 21.0. HRMS (EI+) calculated for C₁₉H₂₁O₃F₃ [M+]: 354.1443. found 354.1429.

### Examples 18-19

Characterization data for AROCM product Aryl ether S4. The acetate of aryl ether 9e was removed and the resultant alcohol was acylated with p-nitro benzoyl chloride to afford S4 and S5, which were separable by pTLC.

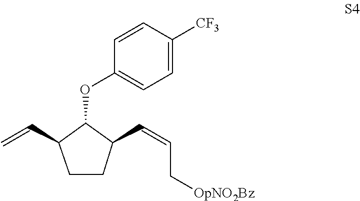

[α]_{D}²⁵-61.96° (c=0.23, CHCl₃); ¹H NMR (500 MHz, CDCl₃) δ 8.30-8.21 (m, 2H), 8.14-8.05 (m, 2H), 7.43 (d, J=8.2 Hz, 2H), 6.91 (d, J=8.8 Hz, 2H), 5.85 (ddd, J=17.1, 10.3, 7.8 Hz, 1H), 5.78-5.66 (m, 2H), 5.10 (ddd, J=17.2, 1.4 Hz, 1H), 5.05 (dd, J=10.3, 1.3 Hz, 1H), 4.98 (dd, J=12.7, 6.4 Hz, 1H), 4.80 (dd, J=12.6, 5.4 Hz, 1H), 4.28 (t, J=5.9 Hz, 1H), 3.19 (m, 1H), 2.86-2.76 (m, 1H), 2.17-2.00 (m, 2H), 1.75-1.64 (m, 1H), 1.64-1.57 (m, 1H). ¹³C NMR (125 MHz, CDCl₃) δ 164.6, 139.2, 137.5, 135.6, 130.8, 126.8, 124.3, 123.6, 116.0, 115.9, 89.2, 61.8, 49.9, 44.8, 30.1, 29.1. HRMS (EI+) calculated for C₂₄H₂₂O₅NF₃ [M+]: 461.1450. found 461.1449.

Separation conditions: OJ-H, 4% IPA, 3.5 mL/min, 95% ee

Racemate:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 5.863 | BV | 0.1692 | 2310.27148 | 202.36076 | 49.8647 |
| 2 | 6.896 | VV | 0.2010 | 2322.81299 | 172.60132 | 50.1353 |

| | | |
|---|---|---|
| Totals: | 4633.08447 | 374.96208 |

Enantioenriched:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 5.889 | VV B | 0.1327 | 118.78459 | 13.23087 | 2.4089 |
| 2 | 6.838 | VV | 0.1879 | 4812.26074 | 379.55093 | 97.5911 |

| | | |
|---|---|---|
| Totals: | 4931.04533 | 392.78181 |

Characterization data for AROCM product Aryl ether S5.

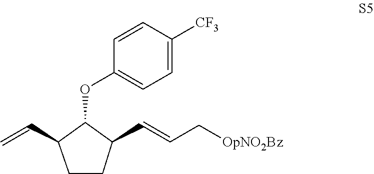

[α]_{D}²⁵+13.44° (c=0.12, CHCl₃); ¹H NMR (500 MHz, CDCl₃) δ 8.31-8.26 (m, 2H), 8.20-8.15 (m, 2H), 7.46 (d, J=8.3 Hz, 1H), 6.93 (d, J=8.2 Hz, 2H), 5.91 (dd, J=15.4, 8.0 Hz, 1H), 5.85 (ddd, J=17.2, 10.3, 7.8 Hz, 1H), 5.75 (ddd, J=15.4, 6.4 Hz, 1H), 5.09 (dd, J=17.2, 1.4 Hz, 1H), 5.08-5.01 (m, 1H), 4.81 (br, 1H), 4.79 (br, 1H), 4.31 (t, J=5.6 Hz, 1H), 2.82 (m, 2H), 2.05 (m, 2H), 1.72-1.61 (m, 2H). ¹³C NMR (125 MHz, CDCl₃) δ 164.5, 139.4, 138.0, 130.8, 126.8, 124.4, 123.7, 116.2, 115.7, 88.6, 66.3, 49.8, 49.1, 29.2, 29.1. HRMS (EI+) calculated for C₂₄H₂₂O₅NF₃ [M+]: 461.1450. found 461.1460.

Separation conditions: OJ-H, 4% IPA, 3.5 mL/min, 95% ee

Racemate:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 6.073 | FM | 0.2053 | 2256.75928 | 183.23097 | 51.8365 |
| 2 | 6.550 | MF | 0.2055 | 2096.85425 | 170.07474 | 48.1635 |

| | | |
|---|---|---|
| Totals: | 4353.61353 | 353.30571 |

Enantioenriched:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 6.141 | VV | 0.1522 | 131.70372 | 13.22259 | 2.6541 |
| 2 | 6.545 | VBA | 0.1761 | 4830.48975 | 414.04193 | 97.3459 |

| | | |
|---|---|---|
| Totals: | 4962.19347 | 427.26452 |

### Substrates for Catalyzed AROCM of Monocyclic Olefins and α-Olefins

Substrates for AROCM were synthesized as previously reported in the literature: substrate 10 (see W. Kirmse, F. Scheidt, H-J. Vater, J. Am. Chem. Soc., 1978, 100, 3945), substrate 13a (see A. H. Hoveyda, P. J. Lombardi, R. V. O'Brien, A. R. Zhugralin, J. Am. Chem. Soc. 2009, 131, 8378), substrate 13b (see (a) T. Mukaiyama, N. Iwasawa, Chem. Lett. 1984, 753-756; (b) D. A. Evans, J. R. Gage, J. L. Leighton, A. S. Kim, J. Org. Chem. 1992, 57, 1961-1963; (c) W. Notz, B. List, J. Am. Chem. Soc. 2000, 122, 7386-7387; (d) M. T. Crimmins, P. J. McDougall, Org. Lett. 2003, 5, 591-594; (e) A. B. Northrup, D. W. C. MacMillan, Science 2004, 305, 1752-1755; (f) A. B. Northrup, I. K. Mangion, F. Hettche, D. W. C. MacMillan, Angew. Chem. 2004, 116, 2204-2206; Angew. Chem., Int. Ed. 2004, 43, 2152-2154; (g) S. E. Denmark, W.-J. Chung, Angew. Chem. 2008, 120, 1916-1918; Angew. Chem., Int. Ed. 2008, 47, 1890-1892), substrate 13c (see R. Gandolfi, M. Ratti, L. Toma, C. De Micheli, Heterocycles 1979, 12, 897), substrate 13d (see A. H. Hoveyda, R. Khan, M. Kashif, P. J. Lombardi, R. V. O'Brien, S. Torker, A. R. Zhugralin, J. Am. Chem. Soc. 2012, 134, 12438) were synthesized according to the provided references. Catalyst 4 was synthesized as previously reported (see J. Hartung, R. H. Grubbs, J. Am. Chem. Soc. 2013, 135, 10183).

### Representative Procedure for Catalyzed AROCM of Monocyclic Olefins and α-Olefins

In a glovebox, cyclobutene 10 (26.6 mg, 0.1 mmol, 1 equiv) and allyl benzoate (14b, 113 mg, 0.7 mmol, 7 equiv) were dissolved in 0.15 mL THF. To this solution was added 50 μL of a stock solution (0.02 M in THF) of catalyst 4. The reaction vial was capped and stirred for 1.5 h and then quenched with an excess of ethyl vinyl ether. The reaction mixture was concentrated and Z/E ratios were determined by 500 MHz ¹H NMR (products 15a-c, 15e-k) or GC (product 12). The crude was subjected to flash chromatography or preparative TLC to afford the desired AROCM product (15f, 25.9 mg, 61% isolated yield, 88:12 Z/E, 97% ee (Z), 88% ee (E)). Pure products (or E/Z mixtures in the case of 15i, and E-15j) were submitted to analytical SFC to determine enantiomer excess.

### Example 20

Characterization data for AROCM product Acetate 12, 79% yield (GC), 85% Z. Z-12. [α]_{D}²⁵-9.34° (c=0.52, CHCl₃); ¹H NMR (500 MHz, CDCl₃) δ 7.37-7.24 (m, 10H), 5.88-5.77 (2×m, 1H), 5.71-5.64 (m, 1H), 5.34 (m, 1H), 5.29 (m, 1H), 4.64 (AB d, J=10.5 Hz, 1H), 4.63 (AB d, J=10.5 Hz, 1H), 4.61 (m, 1H), 4.51-4.46 (m, 1H), 4.45 (AB d, J=10.5 Hz, 1H), 4.43 (AB d, J=10.5 Hz, 1H), 4.21 (ddd, J=9.1, 5.0, 1.0 Hz, 1H), 3.87 (dd, J=7.5, 5.0 Hz, 1H), 2.04 (s, 3H). ¹³C NMR (125 MHz, CDCl₃) δ 170.8, 138.6, 138.4, 135.5, 131.9, 128.5, 128.4, 128.4, 127.8, 127.7, 127.7, 127.5, 119.2, 82.2, 76.6, 70.7, 70.6, 60.8, 21.1. HRMS (FAB+) calculated for C₂₃H₂₇O₄ [M+H]: 367.1909. found 367.1904.

Separation conditions for Z-12: OJ-H, 5% IPA, 2.5 mL/min. 95% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 7.607 | VV | 0.4141 | 5907.10059 | 216.66869 | 49.4689 |
| 2 | 10.124 | VB | 0.5585 | 6033.94629 | 160.53769 | 50.5311 |

| | | |
|---|---|---|
| Totals: | 1.19410e4 | 377.20638 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 7.955 | BV | 0.2626 | 264.61841 | 14.94419 | 2.6277 |
| 2 | 10.319 | BV | 0.3029 | 9805.57031 | 456.01086 | 97.3723 |

| | | |
|---|---|---|
| Totals: | 1.00702e4 | 470.95505 |

E-12. [α]_{D}²⁵-11.8° (c=0.24, CHCl₃); ¹H NMR (500 MHz, CDCl₃) δ 7.36-7.24 (m, 10H), 5.88-5.74 (3×m, 1H), 5.33 (m, 1H), 5.29 (m, 1H), 4.65 (AB d, J=9.3 Hz, 1H), 4.63 (AB d, 9.3 Hz, 1H), 4.61 (d, J=6.0 Hz, 2H), 4.45 (AB d, J=10.6 Hz, 1H), 4.43 (AB d, J=10.7 Hz, 1H), 3.89 (dd, J=6.4, 5.1 Hz, 1H), 3.85 (dd, J=7.2, 5.1 Hz, 1H), 2.08 (s, 3H). ¹³C NMR (125 MHz, CDCl₃) δ 170.9, 138.41, 138.33, 135.5, 131.7, 128.46, 128.45, 128.40, 127.8, 127.75, 127.6, 127.55, 119.1, 82.4, 81.3, 70.9, 70.6, 64.4, 21.1. HRMS (FAB+) calculated for C₂₃H₂₇O₄ [M+H]: 367.1909. found 367.1922.

Separation conditions for E-12: OJ-H, 7% IPA, 2.5 mL/min. 85% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 7.778 | VV | 0.2344 | 2366.55688 | 148.09634 | 50.1123 |
| 2 | 8.429 | VB | 0.2563 | 2355.94971 | 137.19626 | 49.8877 |

| | | |
|---|---|---|
| Totals: | 4722.50659 | 285.29260 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 7.842 | BV | 0.2188 | 764.89539 | 48.82001 | 7.2443 |
| 2 | 8.336 | VB | 0.2556 | 9793.63672 | 540.08466 | 92.7557 |

| | | |
|---|---|---|
| Totals: | 1.05585e4 | 588.90466 |

### Example 21

Characterization data for AROCM product Silyl ether 15a, 66% isolated yield, 88% Z (see S. Saito, H. Itoh, Y. Ono, K. Nishioka, T. Moriwake, Tetrahedron: Asymmetry 1993, 4, 5). Z-15a: [α]_{D}²⁵+4.72° (c=1.06, CHCl₃); ¹H NMR (500 MHz, CDCl₃) δ 5.84 (ddd, J=17.3, 10.4, 6.4 Hz, 1H), 5.80-5.75 (m, 1H), 5.49 (dddd, J=11.2, 8.9, 1.7, 1.1 Hz, 1H), 5.23 (ddd, J=17.3, 1.8, 1.2 Hz, 1H), 5.16 (ddd, J=10.4, 1.8, 1.0 Hz, 1H), 4.34 (ddd, J=8.9, 7.0, 1.1 Hz, 1H), 4.15 (m, 2H), 3.90 (ddt, J=7.3, 6.4, 1.1 Hz, 1H), 2.31 (br, 1H), 0.88 (s, 9H), 0.86 (s, 9H), 0.05 (s, 3H), 0.03 (s, 3H), 0.02 (s, 3H), 0.01 (s, 3H). ¹³C NMR (125 MHz, CDCl₃) δ 139.3, 134.4, 130.3, 116.5, 77.5, 72.8, 59.3, 26.1, 25.9, 18.5, 18.3, −4.2, −4.2, −4.3, −4.5. HRMS (EI+) calculated for C₁₉H₄₁O₃Si₂ [M+H]: 375.2594. found 375.2583.

Z-15a was derivatized by benzoylation and subsequent desilylation to afford a product spectroscopically identical to Z-15b prior to chiral SFC analysis, which indicated 99% ee (see directly below for racemic trace).

Enantioenriched:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.799 | BB | 0.1373 | 1.01426e4 | 1167.74316 | 99.7523 |
| 2 | 4.960 | BV | 0.1919 | 25.18901 | 1.83767 | 0.2477 |

| | | |
|---|---|---|
| Totals: | 1.01678e4 | 1169.58084 |

### Example 22

Characterization data for AROCM product Diol 15b, 67% isolated yield, 75% Z. Z-15b: [α]_{D}²⁵-30.7° (c=0.60, CHCl₃); ¹H NMR (500 MHz, CDCl₃) δ 8.06-8.01 (m, 2H), 7.60-7.54 (m, 1H), 7.47-7.41 (m, 2H), 5.89 (ddd, 17.3, 10.5, 6.2 Hz, 1H), 5.93-5.76 (2×m, 1H), 5.38 (ddd, J=17.3, 1.5, 1.4 Hz, 1H), 5.28 (ddd, J=10.6, 1.5, 1.4 Hz, 1H), 5.08 (ddd, J=12.9, 7.7, 0.8 Hz, 1H), 4.83 (ddd, J=12.6, 5.5, 1.0 Hz, 1H), 4.63 (dd, J=8.0, 4.3 Hz, 1H), 4.25 (ddt, J=6.8, 4.3, 1.3 Hz, 1H), 2.85 (br, 1H), 2.34 (br, 1H). ¹³C NMR (125 MHz, CDCl₃) δ 166.9, 136.0, 133.3, 132.5, 130.0, 129.8, 128.6, 127.7, 118.0, 75.5, 70.4, 61.3. HRMS (EI+) calculated for C₁₄H₁₇O₄ [M+H]: 249.1127. found 249.1117.

Separation conditions for Z-15b: OD-H, 20% IPA, 2.5 mL/min. 91% ee

Racemate:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.777 | BB | 0.1325 | 2620.65259 | 304.35648 | 50.0092 |
| 2 | 4.670 | BB | 0.2558 | 2619.68433 | 144.28246 | 49.9908 |

| | | |
|---|---|---|
| Totals: | 5240.33691 | 448.63893 |

Enantioenriched:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.819 | BB | 0.1414 | 4247.60645 | 453.13589 | 95.4697 |
| 2 | 4.999 | BB | 0.2382 | 201.56192 | 13.19609 | 4.5303 |

| | | |
|---|---|---|
| Totals: | 4449.16837 | 466.33198 |

Characterization data for AROCM product E-15b. [α]_{D}²⁵-1.57° (c=0.06, CHCl₃); ¹H NMR (500 MHz, CDCl₃) δ 8.08-8.01 (m, 2H), 7.60-7.54 (m, 1H), 7.48-7.41 (m, 2H), 6.02 (dtd, J=15.7, 5.7, 1.3 Hz, 1H), 5.96-5.77 (m, 2H), 5.37 (ddd, J=17.3, 1.5, 1.4 Hz, 1H), 5.29 (ddd, J=10.6, 1.5, 1.4 Hz, 1H), 5.07 (m, 1H), 4.87 (m, 1H), 4.68 (m, 1H), 4.25 (m, 1H), 2.89 (br, 1H), 2.00 (br, 1H). ¹³C NMR (125 MHz, CDCl₃) δ 166.8, 135.9, 133.3, 132.5, 130.1, 129.8, 128.6, 127.9, 118.0, 75.6, 70.3, 61.2.

Separation conditions for E-15b: OJ-H, 20% IPA, 2.5 mL/min. 67% ee

Racemate:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.136 | BV | 0.1100 | 971.22040 | 138.18152 | 48.7580 |
| 2 | 3.435 | VV | 0.1207 | 1020.70038 | 134.19174 | 51.2420 |

| | | |
|---|---|---|
| Totals: | 1991.92078 | 272.37326 |

Enantioenriched:

Signal 2: DAD1 B, Sig = 235, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.168 | BV | 0.1130 | 686.64221 | 94.23962 | 16.5860 |
| 2 | 3.473 | VV | 0.1246 | 3453.24072 | 434.97318 | 83.4140 |

| | | |
|---|---|---|
| Totals: | 4139.88293 | 529.21280 |

### Example 23

Characterization data for AROCM product Benzoate 15c, 69% isolated yield, 75% Z. Z-15c: [α]_{D}²⁵+4.06° (c=0.95, CHCl₃); ¹H NMR (500 MHz, CDCl₃) δ 8.09-8.04 (m, 2H), 8.02-7.97 (m, 2H), 7.61-7.54 (2×m, 1H), 7.49-7.39 (2×m, 2H), 6.09-5.96 (3×m, 1H), 5.83-5.78 (m, 1H), 5.67 (dd, J=11.0, 9.7 Hz, 1H), 5.52 (d, J=17.3 Hz, 1H), 5.41 (d, J=10.5 Hz, 1H), 4.56 (ddd, J=13.4, 7.8, 1.4 Hz, 1H), 4.20 (ddd, J=13.4, 5.7, 1.2 Hz, 1H). ¹³C NMR (125 MHz, CDCl₃) δ 166.1, 165.6, 135.4, 133.5, 133.4, 131.8, 130.0, 129.9, 129.85, 129.80, 128.6, 128.6, 125.3, 120.4, 75.6, 71.4, 58.8. HRMS (FAB+) calculated for C₂₁H₂₁O₅ [M+H]: 353.1389. found 353.1381.

Separation conditions for Z-15c: OJ-H, 5% IPA, 2.5 mL/min. 96% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 9.799 | BV | 0.2620 | 3595.55029 | 207.71271 | 50.2636 |
| 2 | 11.027 | BB | 0.2878 | 3557.83179 | 188.87053 | 49.7364 |

| | | |
|---|---|---|
| Totals: | 7153.38208 | 396.58324 |

Enatioenriched

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 9.836 | BB | 0.2567 | 6250.97852 | 370.80807 | 97.9546 |
| 2 | 10.889 | BB | 0.2427 | 130.52478 | 6.96354 | 2.0454 |

| | | |
|---|---|---|
| Totals: | 6381.50330 | 377.77162 |

E-15c. [α]_{D}²⁵-1.14° (c=0.56, CHCl₃); ¹H NMR (500 MHz, CDCl₃) δ 8.10-7.97 (2×m, 2H), 7.60-7.52 (2×m, 1H), 7.48-7.39 (2×m, 2H), 6.10 (ddd, 15.5, 4.9, 4.8 Hz, 1H), 6.02 (ddd, 17.3, 10.6, 6.4 Hz, 1H), 5.92 (dddd, 15.4, 6.9, 1.7, 1.6 Hz, 1H), 5.84 (m, 1H), 5.80 (m, 1H), 5.49 (d, J=17.2 Hz, 1H), 5.39 (d, J=10.5 Hz, 1H), 4.24-4.18 (m, 2H). ¹³C NMR (125 MHz, CDCl₃) δ 165.6, 165.5, 135.2, 133.3, 131.8, 130.1, 129.9, 128.6, 128.6, 124.4, 120.1, 75.7, 74.9, 62.8. HRMS (FAB+) calculated for C₂₁H₁₉O₄ [M−OH]: 335.1283. found 335.1271.

Separation conditions for E-15c: OJ-H, 5% IPA, 2.5 mL/min. 82% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 12.117 | BB | 0.2964 | 2167.36914 | 110.68974 | 49.1751 |
| 2 | 13.450 | BB | 0.3262 | 2240.08228 | 102.82623 | 50.8249 |

| | | |
|---|---|---|
| Totals: | 4407.45142 | 213.51597 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.841 | VV | 0.3037 | 2009.88196 | 101.20198 | 90.8688 |
| 2 | 13.043 | BB | 0.3044 | 201.96896 | 9.96831 | 9.1312 |

| | | |
|---|---|---|
| Totals: | 2211.85092 | 111.17029 |

### Example 24

Characterization data for AROCM product Alcohol 15e, 62% isolated yield, 89% Z. Z-15e: [α]_{D}²⁵-2.95° (c=0.76, CHCl₃); ¹H NMR (500 MHz, CDCl₃) δ 7.37-7.24 (m, 10H), 6.02 (ddd, J=11.1, 6.9, 6.8 Hz, 1H), 5.83 (ddd, J=17.6, 10.4, 7.5 Hz, 1H), 5.56 (dd, J=11.5, 8.9 Hz, 1H), 5.39 (m, 1H), 5.37-5.32 (m, 1H), 4.64 (AB d, J=10.5 Hz, 1H), 4.62 (AB d, J=11.0 Hz, 1H), 4.42 (AB d, J=12.1 Hz, 1H), 4.38 (AB d, J=11.7 Hz, 1H), 4.21 (dd, J=8.6, 7.4, 1.0 Hz, 1H), 4.07-3.93 (2×m, 1H), 3.78 (dd, J=7.2, 7.0 Hz, 1H), 2.13 (br, 1H). ¹³C NMR (125 MHz, CDCl₃) δ 138.2, 137.7, 135.8, 133.7, 131.6, 128.5, 128.4, 128.2, 127.9, 127.8, 127.7, 119.5, 81.5, 76.3, 70.8, 70.7, 58.5. HRMS (FAB+) calculated for C₂₁H₂₅O₃ [M+H]: 325.1804. found 325.1803.

Separation conditions for Z-15e: OJ-H, 10% IPA, 2.5 mL/min. 93% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.281 | BV | 0.2773 | 3639.78735 | 202.93092 | 49.9668 |
| 2 | 12.022 | VB | 0.3028 | 3644.62769 | 187.47997 | 50.0332 |

| | | |
|---|---|---|
| Totals: | 7284.41504 | 390.41089 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.220 | BV | 0.2596 | 210.15450 | 10.58754 | 3.5605 |
| 2 | 11.938 | VV | 0.3010 | 5692.16504 | 295.11295 | 96.4395 |

| | | |
|---|---|---|
| Totals: | 5902.31953 | 305.70049 |

E-15e. [α]_{D}²⁵-2.93° (c=0.30, CHCl₃); ¹H NMR (500 MHz, CDCl₃) δ 7.36-7.23 (m, 10H), 5.93-5.79 (2×m, 1H), 5.71 (ddd, J=15.7, 7.5, 7.3 Hz, 1H), 5.33 (m, 1H), 5.29 (m, 1H), 4.65 (AB d, J=12.2 Hz, 1H), 4.62 (AB d, J=12.2 Hz, 1H), 4.47 (AB d, J=12.2 Hz, 1H), 4.43 (AB d, J=12.1 Hz, 1H), 4.18 (m, 2H), 3.90 (dd, J=7.9, 5.6 Hz, 1H), 3.86 (ddd, J=7.4, 4.8, 0.9 Hz, 1H). ¹³C NMR (125 MHz, CDCl₃) δ 138.7, 138.6, 135.6, 133.7, 128.8, 128.4, 127.9, 127.8, 127.6, 127.5, 119.0, 82.5, 81.6, 70.8, 70.7, 63.2. HRMS (FAB+) calculated for C₂₁H₂₅O₃ [M+H]: 325.1804. found 325.1812.

Separation conditions for E-15e: OJ-H, 10% IPA, 2.5 mL/min. 86% ee

Racemate:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 6.977 | BV | 0.1991 | 1499.56213 | 109.99121 | 50.4390 |
| 2 | 7.720 | VV | 0.2202 | 1473.45618 | 97.57201 | 49.5610 |
| Totals: | | | | 2973.01831 | 207.56322 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 7.071 | VV | 0.2045 | 342.74240 | 24.91814 | 6.9119 |
| 2 | 7.788 | VB | 0.2345 | 4615.99463 | 288.76001 | 93.0881 |
| Totals: | | | | 4958.73703 | 313.67815 | |

### Example 25

Characterization data for AROCM product Benzoate 15f, 61% isolated yield, 88% Z. Z-15f: [α]_{D}²⁵-50.9° (c=0.74, CHCl₃); ¹H NMR (500 MHz, CDCl₃) δ 8.08-8.02 (m, 2H), 7.60-7.54 (m, 1H), 7.47-7.41 (m, 2H), 7.37-7.22 (m, 10H), 5.97 (dddd, J=11.3, 7.8, 5.8, 1.1 Hz, 2H), 5.85 (ddd, J=17.1, 10.5, 7.5 Hz, 1H), 5.73 (ddd, J=10.7, 9.2, 1.5 Hz, 1H), 5.35-5.33 (m, 1H), 5.31 (m, 1H), 4.87 (ddd, J=13.2, 7.8, 1.4 Hz, 1H), 4.73 (ddd, J=13.2, 5.8, 1.6 Hz, 2H), 4.68 (AB d, J=12.2 Hz 1H), 4.64 (AB d, J=12.1 Hz, 1H), 4.49 (AB d, J=12.1 Hz, 1H), 4.44 (AB d, J=12.2 Hz, 1H), 4.30 (ddd, J=9.1, 5.0, 1.1 Hz, 2H), 3.90 (dd, J=7.5, 5.0 Hz, 1H). ¹³C NMR (125 MHz, CDCl₃) δ 166.4, 138.6, 138.4, 135.5, 133.1, 132.1, 130.2, 129.7, 128.55, 128.50, 128.45, 128.40, 127.8, 127.75, 127.70, 127.5, 119.2, 82.3, 76.7, 70.7, 70.7, 61.2. HRMS (FAB+) calculated for C₂₈H₂₉O₄ [M+H]: 429.2066. found 429.2056.

Separation conditions for Z-15f: OJ-H, 20% IPA, 2.5 mL/min. 97% ee

Racemate:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 4.059 | VV | 0.1335 | 8624.85742 | 991.43774 | 49.2384 |
| 2 | 4.842 | VB | 0.1622 | 8891.65625 | 849.16925 | 50.7616 |
| Totals: | | | | 1.75165e4 | 1840.60699 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 4.088 | BV | 0.2156 | 537.15137 | 38.29383 | 1.7991 |
| 2 | 4.857 | VB | 0.2960 | 2.93192e4 | 1527.33167 | 98.2009 |
| Totals: | | | | 2.98564e4 | 1565.62550 | |

E-15f. ¹H NMR (500 MHz, CDCl₃) δ 8.08-8.04 (m, 2H), 7.61-7.54 (m, 1H), 7.45 (m, 2H), 7.36-7.21 (m, 10H), 5.98-5.79 (3×m, 1H), 5.34 (m, 1H), 5.29 (m, 1H), 4.87 (2×m, 1H), 4.64 (AB d, J=12.0 Hz, 2H), 4.47 (AB d, J=12.1 Hz, 1H), 4.43 (AB d, J=12.1 Hz, 1H), 3.92 (dd, J=6.8, 5.3 Hz, 1H), 3.87 (dd, J=6.8, 5.5 Hz, 1H). ¹³C NMR (125 MHz, CDCl₃) δ 166.4, 138.50, 138.42, 135.6, 133.1, 131.8, 130.1, 129.82, 129.80, 128.55, 128.52, 128.44, 128.36, 127.8, 127.60, 127.56, 119.1, 82.4, 81.3, 70.9, 70.6, 64.8.

Separation conditions for E-15f: OD-H, 20% IPA, 2.5 mL/min. 88% ee

Racemate:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 5.665 | BV | 0.1888 | 5934.12598 | 478.10345 | 49.9106 |
| 2 | 6.250 | VB | 0.2143 | 5955.37939 | 428.19113 | 50.0894 |
| Totals: | | | | 1.18895e4 | 906.29459 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 4.925 | BB | 0.2038 | 401.86090 | 31.71189 | 4.3113 |
| 2 | 5.599 | BV | 0.2213 | 536.10181 | 38.78575 | 5.7514 |
| 3 | 6.183 | VB | 0.2409 | 8383.21680 | 552.90784 | 89.9373 |
| Totals: | | | | 9321.17950 | 623.40548 | |

### Example 26

Characterization data for AROCM product Silyl ether 15g, 68% yield, 87% Z. Initial product mixture derivatized by treatment with TBAF (3 equiv) to aid in purification; isolated product is spectroscopically identical to alcohol 15e.

Optical rotations and enantiopurity of derivatized products:

Derivative of Z-15g: [α]_{D}²⁵-2.2° (c=0.61, CHCl₃)

89% ee

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 11.261 | VV | 0.3050 | 316.99796 | 14.38933 | 5.7900 |
| 2 | 11.972 | VV | 0.3092 | 5157.96875 | 262.54877 | 94.2100 |
| Totals: | | | | 5474.96671 | 276.93809 | |

Derivative of E-15g:[α]_{D}²⁵-3.4°(c=0.31, CHCI₃)

77% ee

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 7.070 | VV | 0.2254 | 329.41394 | 21.65948 | 11.2280 |
| 2 | 7.819 | VB | 0.2454 | 2604.45679 | 157.07635 | 88.7720 |
| Totals: | | | | 2933.87073 | 178.73583 | |

### Example 27

Characterization data for AROCM product Benzyl ether 15h, 64% isolated yield, 86% Z. Z-15h: [α]_{D}²⁵-29.7° (c=0.66, CHCl₃); ¹H NMR (500 MHz, CDCl₃) δ 7.36-7.23 (m, 10H), 5.91 (dddd, J=11.4, 7.3, 5.4, 1.1 Hz, 1H), 5.83 (ddd, J=17.2, 10.4, 7.6 Hz, 1H), 5.61 (dddd, J=11.0, 9.2, 1.7, 1.6 Hz, 1H), 5.34-5.30 (m, 1H), 5.28 (m, 1H), 4.64 (AB d, J=12.2 Hz, 1H), 4.61 (AB d, J=12.1 Hz, 1H), 4.43 (AB d, J=12.2 Hz, 1H), 4.43-4.41 (2×AB d, 1H), 4.40 (AB d, J=12.1 Hz, 1H), 4.16 (ddd, J=9.2, 4.9, 1.1 Hz, 1H), 4.04 (ddd, J=12.6, 7.3, 1.6 Hz, 1H), 3.93 (ddd, J=12.6, 5.4, 1.8 Hz, 1H), 3.82 (dddd, J=7.6, 5.0, 1.2, 0.9 Hz, 1H). ¹³C NMR (125 MHz, CDCl₃) δ 138.6, 138.5, 138.3, 135.5, 131.6, 130.3, 128.52, 128.39, 128.36, 127.84, 127.81, 127.77, 127.76, 127.56, 127.53, 119.1, 82.5, 76.4, 72.5, 70.6, 70.4, 66.4. HRMS (FAB+) calculated for C₂₈H₃₁O₃ [M+H]: 415.2273. found 415.2260.

Separation conditions for Z-15h: OD-H, 15% IPA, 2.5 mL/min. 91% ee

Racemate:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 7.714 | BB | 0.2523 | 3691.89014 | 233.73363 | 49.8466 |
| 2 | 8.793 | VB | 0.2827 | 3714.60791 | 205.71669 | 50.1534 |
| Totals: | | | | 7406.49805 | 439.45032 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 7.739 | BV | 0.2524 | 6725.33252 | 416.38754 | 95.2967 |
| 2 | 8.821 | VB | 0.2569 | 331.92462 | 18.54005 | 4.7033 |
| Totals: | | | | 7057.25714 | 434.92760 | |

### Example 28

Characterization data for AROCM product 15i. Isolated as an inseparable 9:1 Z/E mixture, 76% yield. Z-15i: ¹H NMR (500 MHz, CDCl₃) δ 7.38-7.25 (m, 10H), 7.06-7.00 (m, 2H), 6.79-6.75 (m, 2H), 5.95-5.82 (2×m, 1H), 5.54 (ddd, J=11.0, 9.4, 1.7, 1.5 Hz, 1H), 5.37 (m, 1H), 5.29 (m, 1H), 4.67 (2×AB d, J=12.2 Hz, 2H), 4.49 (AB d, J=12.2 Hz, 1H), 4.47 (AB d, J=12.1 Hz, 1H), 4.36 (ddd, J=9.3, 4.8, 1.1 Hz, 1H), 3.89 (dd, J=7.7, 4.9 Hz, 1H), 3.78 (s, 3H), 3.34-3.20 (m, 2H). ¹³C NMR (125 MHz, CDCl₃) δ 158.0, 138.77, 138.76, 135.7, 133.9, 132.4, 129.63, 129.45, 128.4, 128.0, 127.84, 127.78, 127.53, 127.49, 119.0, 114.0, 82.7, 76.3, 70.6, 70.3, 55.4, 33.4. HRMS (FAB+) calculated for C₂₈H₃₁O₃ [M+H]: 415.2273. found 415.2287.

Separation conditions for Z/E product mixture: AD-H, 10% IPA, 2.5 mL/min. Z: 93% ee; E: 79% ee.

Racemate:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 9.466 | VB | 0.2176 | 4234.35059 | 305.74072 | 44.0459 |
| 2 | 10.421 | BV | 0.2378 | 4251.52734 | 279.00278 | 44.2245 |
| 3 | 10.869 | VV | 0.2464 | 545.18933 | 34.12083 | 5.6711 |
| 4 | 12.217 | VV | 0.2952 | 582.43701 | 31.58193 | 6.0585 |
| Totals: | | | | 9613.50427 | 650.44626 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 8.802 | BV | 0.2556 | 2.11839e4 | 1345.92456 | 85.3335 |
| 2 | 9.623 | VV | 0.2273 | 735.39277 | 48.94960 | 2.9623 |
| 3 | 9.977 | VB | 0.2593 | 298.80161 | 16.18907 | 1.2036 |
| 4 | 11.232 | BV | 0.2637 | 2606.73169 | 152.29791 | 10.5005 |
| Totals: | | | | 2.48248e4 | 1563.36114 | |

### Example 29

Characterization data for AROCM Ketone 15j, 65% isolated yield, 90% Z. Z-15j: [α]_{D}²⁵-7.98° (c=1.35, CHCl₃); ¹H NMR (500 MHz, CDCl₃) δ 7.39-7.22 (m, 10H), 5.86 (ddd, J=17.2, 10.4, 7.6 Hz, 1H), 5.65 (dtd, J=11.1, 7.5, 1.0 Hz, 1H), 5.46 (ddt, J=10.9, 9.3, 1.6 Hz, 1H), 5.35 (m, 1H), 5.27 (m, 1H), 4.66 (AB d, J=12.1 Hz, 1H), 4.61 (AB d, J=12.2 Hz, 1H), 4.45 (AB d, J=12.1 Hz, 1H), 4.43 (AB d, J=12.2 Hz, 1H), 4.23 (ddd, J=9.3, 5.0, 1.0 Hz, 1H), 3.84 (dd, J=7.6, 5.0, 1H), 2.38 (m, 2H), 2.24 (m, 2H), 2.04 (s, 3H). ¹³C NMR (125 MHz, CDCl₃) δ 208.0, 138.753, 138.746, 135.7, 133.2, 128.6, 128.36, 128.34, 127.81, 127.75, 127.51, 127.49, 118.9, 82.6, 76.3, 70.6, 70.3, 43.3, 30.0, 22.3. HRMS (FAB+) calculated for C₂₄H₂₉O₃ [M+H]: 365.2117. found 365.2113.

Separation conditions for Z-15j: OJ-H, 5% IPA, 2.5 mL/min. 92% ee

Racemate:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 9.787 | BV | 0.3728 | 4472.42529 | 180.68672 | 49.6322 |
| 2 | 10.883 | VBA | 0.4203 | 4538.71436 | 163.26811 | 50.3678 |
| Totals: | | | | 9011.13965 | 343.95483 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 9.565 | BB | 0.2466 | 294.21240 | 18.00591 | 3.8669 |
| 2 | 10.607 | BB | 0.2664 | 7314.22949 | 405.54443 | 96.1331 |
| Totals: | | | | 7608.44189 | 423.55034 | |

E/Z-15j mixture:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU*s] | [mAU] | % |
| 1 | 9.874 | BB | 0.2847 | 2521.13306 | 133.25986 | 16.6689 |
| 2 | 10.986 | BV | 0.3148 | 2570.48706 | 123.50429 | 16.9952 |
| 3 | 11.655 | VB | 0.3314 | 5003.31738 | 226.75299 | 33.0803 |
| 4 | 12.990 | BB | 0.3684 | 5029.84863 | 206.30301 | 33.2557 |
| Totals: | | | | 1.51248e4 | 689.82014 | |

Enantioenriched: E84% ee.

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 9.616 | BV | 0.2410 | 174.28995 | 10.11655 | 1.9790 |
| 2 | 10.689 | VV | 0.2750 | 3897.29248 | 211.45294 | 44.2520 |
| 3 | 11.391 | VB | 0.2866 | 379.96729 | 19.56160 | 4.3144 |
| 4 | 12.698 | BV | 0.3234 | 4355.48779 | 202.13602 | 49.4546 |

| | | |
|---|---|---|
| Totals: | 8807.03751 | 443.26711 |

### Example 30

Characterization data for AROCM Boronic ester 15k, 50% isolated yield of Z product. [α]_{D}²⁵ 7.98° (c=0.64, CHCl₃); ¹H NMR (500 MHz, CDCl₃) δ 7.37-7.28 (m, 10H), 5.94-5.78 (2×m, 1H), 5.43 (dddd, J=11.0, 9.3, 1.7, 1.5 Hz, 1H), 5.28 (m, 1H), 5.25 (m, 1H), 4.67 (AB d, J=12.2 Hz, 1H), 4.64 (AB d, J=12.3 Hz, 1H), 4.47 (AB d, J=12.4 Hz, 1H), 4.44 (AB d, J=12.2 Hz, 1H), 4.30 (ddd, J=9.4, 4.0, 1.1 Hz, 1H), 3.88 (dd, J=7.7, 4.0 Hz, 1H), 1.69 (m, 2H), 1.23 (s, 6H), 1.22 (s, 6H). ¹³C NMR (125 MHz, CDCl₃) δ 139.1, 139.0, 135.7, 130.0, 128.31, 128.30, 127.7, 127.6, 127.34, 127.33, 126.9, 118.8, 83.5, 82.8, 76.2, 70.5, 70.1, 24.94, 24.93. HRMS (FAB+) calculated for C₂₀H₂₈O₃B [M-OBn]: 327.2132. found 327.2138.

Separation conditions for Z-15k: OJ-H, 5% IPA, 2.5 mL/min. 91% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 4.784 | BV | 0.2705 | 4108.72510 | 219.40474 | 55.4931 |
| 2 | 7.295 | VB | 0.3813 | 3295.29712 | 132.87819 | 44.5069 |

| | | |
|---|---|---|
| Totals: | 7404.02222 | 352.28293 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 4.898 | BV | 0.1625 | 331.67175 | 30.59655 | 4.2149 |
| 2 | 5.190 | VV | 0.2376 | 402.79446 | 23.30932 | 5.1187 |
| 3 | 7.472 | VV | 0.2806 | 7134.57031 | 384.21341 | 90.664 |

| | | |
|---|---|---|
| Totals: | 7869.03653 | 438.11928 |

### Example 31

Characterization data for AROCM Alcohol 17. Alcohol 17 was synthesized following the general AROCM procedure in 85% isolated yield, 91% Z, and 1:1 dr. Z-17: ¹H NMR (500 MHz, CDCl₃) δ 7.36-7.24 (m, 10H), 5.89-5.78 (2×m, 1H), 5.54-5.43 (dddd, J=11.1, 9.8, 1.3, 1.0 Hz, 1H), 5.38 (m, 1H), 5.32 (m, 1H), 4.66 (AB d, J=12.3 Hz, 2H), 4.59 (AB d, J=12.2 Hz, 2H), 4.41 (AB d, J=12.4 Hz, 2H), 4.38 (AB d, J=12.1 Hz, 2H), 4.20 (ddd, J=9.8, 6.9, 0.9 Hz, 2H), 3.78 (dd, J=7.7, 6.9 Hz, 1H), 3.74 (m, 1H), 2.81 (br, 1H), 2.18-2.10 (m, 2H), 1.16 (d, J=6.2 Hz, 3H). ¹³C NMR (125 MHz, CDCl₃) δ 138.5, 137.9, 135.9, 131.8, 131.1, 128.40, 128.37, 128.2, 127.82, 127.75, 127.6, 119.7, 81.2, 75.6, 70.23, 70.18, 66.9, 38.1, 23.2. HRMS (FAB+) calculated for C₂₃H₂₉O₃ [M+H]: 353.2117. found 353.2108.

### Example 32

Ketone 18: Dess-Martin periodinane (302 mg, 0.713 mmol, 2 equiv) was added in one portion to a cold (0° C.) solution of alcohols Z-17 (126 mg, 0.356 mmol) in CH₂Cl₂ (5 mL). The reaction mixture was allowed to warm to room temperature and stirred for 1 h. Aqueous 1:1 NaHCO₃/Na₂S₂O₃ solution was added and the biphasic mixture stirred vigorously for 1 h. The layers were separated, and the aqueous layer extracted with CH₂Cl₂. The combined organic layers were dried over MgSO₄, filtered and concentrated. The crude residue was purified by flash chromatography to afford 110.4 mg, 88% yield of ketone 18. [α]_{D}²⁵-14.4° (c=0.83, CHCl₃); ¹H NMR (500 MHz, CDCl₃) δ 7.36-7.24 (m, 10H), 5.93 (dddd, J=11.1, 10.8, 7.2, 1.1 Hz, 1H), 5.85 (ddd, J=17.2, 10.4, 7.6 Hz, 1H), 5.63 (dddd, J=11.0, 9.1, 1.7, 1.4 Hz, 1H), 5.36-5.33 (m, 1H), 5.33-5.27 (m, 1H), 4.63 (2×ABd, J=12.0 Hz, 2H), 4.43 (AB d, J=10.8 Hz, 1H), 4.39 (AB d, J=Hz, 1H), 4.09 (ddd, J=9.1, 5.2, 1.1 Hz, 1H), 3.84 (dd, J=7.6, 5.3 Hz, 1H), 3.08 (dd, J=7.2, 1.7 Hz, 2H), 2.03 (s, 3H). ¹³C NMR (125 MHz, CDCl₃) δ 206.1, 138.6, 138.4, 135.6, 130.7, 128.40, 128.37, 127.87, 127.86, 127.62, 127.58, 126.4, 119.1, 82.4, 76.3, 70.7, 70.3, 42.7, 29.8. HRMS (FAB+) calculated for C₂₃H₂₇O₃ [M+H]: 351.1960. found 351.1954.

Separation conditions for 18: AD-H, 5% IPA, 2.5 mL/min. 95% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 7.509 | VV | 0.1535 | 2803.53540 | 288.06458 | 49.4751 |
| 2 | 7.938 | VV | 0.1670 | 2863.01855 | 262.90552 | 50.5249 |

| | | |
|---|---|---|
| Totals: | 5666.55396 | 550.97009 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 7.548 | BV | 0.1477 | 159.29933 | 16.64186 | 2.4029 |
| 2 | 7.911 | VV | 0.1731 | 6470.11279 | 584.80743 | 97.5971 |

| | | |
|---|---|---|
| Totals: | 6629.41212 | 601.44930 |

### Example 33

(+)-endo-brevicomin (19). Ketone 18 (35 mg, 0.10 mmol) was dissolved in 5:1 MeOH/1 N HCl (aq.) and the reaction flask purged with Argon. Palladium on carbon (10%, 35 mg) was added, and the flask was purged by a balloon filled with H₂. The reaction mixture was stirred under 1 atm of H₂ for 2 h. The reaction flask was then purged with Argon and Celite was added. The suspension was filtered through Celite and the organic layer was extracted with pentane. The combined pentane layers were washed with water, brine, and dried over MgSO₄. The pentane layers were filtered and carefully concentrated to afford the crude reaction mixture (9.9 mg, 67% yield), containing 90% purity (+)-endo-brevicomin Analytical samples were afforded by flash chromatography. [α]_{D}²⁵+49.6° (c=0.11, CHCl₃), lit. (see G. Pedrocchi-Fantoni, S. Servi, J. Chem. Soc., Perkin. Trans. 1 1991, 1764. [α]_{D}²⁰+49° (c=1.0, ether, 96.5% ee, 90% purity), lit. (see S. Singh, P. J. Guiry, J. Org. Chem. 2009, 74, 5758). [α]_{D}²⁰ 77.9° 9 (c=1.2, ether, 99.3% ee); ¹H NMR (500 MHz, CDCl₃) δ 4.21 (dt, J=4.6, 2.3 Hz, 1H), 3.99 (tdd, J=7.2, 4.1, 1.0 Hz, 1H), 1.99-1.72 (m, 4H), 1.68-1.51 (m, 4H), 1.43 (s, 3H), 0.99 (t, J=7.5 Hz, 3H). ¹³C NMR (125 MHz, CDCl₃) δ 107.0, 81.6, 76.6, 34.4, 25.0, 23.6, 21.9, 17.6, 10.9. HRMS (FAB+) calculated for C₉H₁₇O₂ [M+H]: 157.1229. found 157.1206.

Separation conditions (GC, GTA column): 80° C., isocratic. 96% ee

Racemate:

| Peak | RetTime | | Width | Area | Height | Area |
|---|---|---|---|---|---|---|
| # | [min] | Type | [min] | [pA*s] | [pA] | % |
| 1 | 8.063 | BB | 0.1233 | 30.86736 | 3.62755 | 50.14859 |
| 2 | 8.646 | BB | 0.1302 | 30.68445 | 3.27842 | 49.85141 |

Enantioenriched:

| Peak | RetTime | | Width | Area | Height | Area |
|---|---|---|---|---|---|---|
| # | [min] | Type | [min] | [pA*s] | [pA] | % |
| 1 | 8.098 | BB | 0.1045 | 2.60984 | 3.18731e−1 | 1.90416 |
| 2 | 8.656 | BB | 0.1424 | 120.87816 | 12.66055 | 88.19333 |
| 3 | 9.918 | BB | 0.116 | 13.57243 | 1.55830 | 9.90251 |

### Example 34

Diol 20. To a biphasic mixture of 1:1 tBuOH/water containing diene Z-15g (38.5 mg, 0.089 mmol) was sequentially added potassium carbonate (37 mg, 0.27 mmol), potassium ferricyanide (89 mg, 0.27 mmol, 3 equiv), and potassium osmate dihydrate (1.7 mg, 4.6 μmol, 5 mol %) at 0° C. The reaction was stirred vigorously at 23° C. for 24 h. Upon completion, solid Na₂SO₃ was added stirred continued at 23° C. for 2 h. EtOAc was added and the layers separated. The aqueous layer was extracted with EtOAc and the combined organic layers washed with water, brine, and dried over MgSO₄. After filtration and concentration, the crude residue was subject to flash chromatography to afford 27.5 mg, 66% yield of diol 20.

Major diastereomer: [α]_{D}²⁵-62.1° (c=1.35, CHCl₃); ¹H NMR (500 MHz, CDCl₃) δ 8.05-8.01 (m, 2H), 7.60-7.55 (m, 1H), 7.44 (dd, J=8.5, 7.2 Hz, 2H), 7.37-7.22 (m, 26H), 6.05-5.97 (m, 1H), 5.86-5.78 (m, 1H), 4.89-4.83 (m, 2H), 4.77 (d, J=11.1 Hz, 1H), 4.67 (d, J=11.8 Hz, 1H), 4.65-4.62 (m, 1H), 4.60 (dd, J=9.6, 4.6 Hz, 1H), 4.45 (d, J=11.7 Hz, 1H), 3.72 (dt, J=13.1, 5.0 Hz, 4H). ¹³C NMR (125 MHz, CDCl₃) δ 166.6, 138.1, 137.8, 133.3, 131.3, 129.87, 128.78, 128.65, 128.62, 128.58, 128.3, 128.02, 128.01, 128.0, 80.9, 76.1, 74.6, 72.1, 70.8, 66.3, 63.7, 61.2. HRMS (FAB+) calculated for C₂₈H₃₁O₆ [M+H]: 463.2121. found 463.2125.

### Example 35

Methyl glycoside 21. Diol 20 (34.6 mg, 0.075 mmol) was dissolved in 1:1 CH₂Cl₂/MeOH and cooled to −78° C. Ozone was bubbled through the solution until a blue color persisted for 10 min. At this point, oxygen was bubbled through the solution until the reaction appeared colorless. Excess dimethyl sulfide (0.1 mL) was added and the reaction was allowed to come to room temperature and stir for 16 h. The reaction mixture was concentrated and the crude residue used in the following step. The crude aldehyde was then dissolved in MeOH (5 mL) and cooled to 0° C. HCl in MeOH (0.4 M, 0.5 mL) was added and the reaction was warmed to room temperature. The reaction was stirred for 14 h, at which time Amberlyst IRA-400 (OH⁻) was added. The mixture was filtered and concentrated; preparative TLC afforded 10.6 mg (0.031 mmol, 47% yield over two steps) of methyl glycoside 21. [α]_{D}²⁵=−36.4° (c=0.27, CHCl₃), lit (see P. A. Wender, F. C. Bi, N. Buschmann, F. Gosselin, C. Kan, J-M. Kee, H. Ohmura, Org. Lett. 2006, 8, 5373). ent-21 [α]_{D}²⁵=+31.7 (c=1.94, CHCl₃); ¹H NMR (500 MHz, CDCl₃) δ 7.40-7.27 (m, 10H), 4.89 (s, 1H), 4.66 (AB d, J=12.0 Hz, 1H), 4.63 (AB d, J=12.0 Hz, 1H), 4.58 (AB d, J=11.7 Hz, 1H), 4.49 (AB d, J=11.7 Hz, 1H), 4.28 (m, 1H), 4.13 (dd, J=7.1, 4.7 Hz, 1H), 3.87 (d, J=4.7 Hz, 1H), 3.83-3.77 (m, 1H), 3.58 (m, 1H), 3.37 (s, 3H), 1.95 (br, 1H). ¹³C NMR (125 MHz, CDCl₃) δ 137.81, 137.79, 128.6, 128.1 (4C), 128.04 (3C), 128.00 (3C), 107.0, 82.4, 80.3, 77.4, 72.8, 72.6, 62.8, 55.7. HRMS (FAB+) calculated for C₂₀H₂₃O₅ [M+H−H₂]: 343.1545. found 343.1553.

### General Procedure for Preparation of Silver Carboxylates

Following a known procedure (see Dorta, R.; Shimon, L.; Milstein, D. J. Organomet. Chem. 2004, 689, 751-758) L-N-acetyl alanine (200 mg, 1.53 mmol, 2 equiv.) was added to a stirring suspension of silver oxide (177 mg, 0.762 mmol, 1 equiv.) in 4 mL acetonitrile shielded from light. The reaction was vigorously stirred for 24 h, at which time a light gray precipitate had formed. The mixture was filtered and washed with acetonitrile and ether. The resultant solid was dried under vacuum overnight while shielded from light to provide 212 mg (0.89 mmol, 58% yield) of silver carboxylate.

### General Procedure for Preparation of Catalysts 22a-h

To a solution of enantiopure ruthenium iodide 1 (1.92 mg, 0.0028 mmol) in 0.5 mL THF was added silver carboxylate from above (1.3 mg, 0.055 mmol, 2 equiv.). The mixture was stirred for 30 min and then concentrated. The resultant solid was redissolved in benzene and filtered through a short pad of Celite. The resultant purple solution was concentrated, assayed by ¹H NMR and then used directly in the AROCM reaction. ¹NMR spectra of complexes 22a-c matched previously reported spectra of the corresponding racemic complexes (see Keitz, B. K.; Endo, K.; Patel, P. R.; Herbert, M. B.; Grubbs, R. H. J. Am. Chem. Soc., 2012, 134, 693-699). Diagnostic benzylidene signals (C₆D₆) of novel compounds are listed below:

22d: 14.99 ppm
22e: 15.00 ppm
22f: 15.10 ppm
22h: 15.11 ppm

### Preparation of Substrates for AROCM

Substrates for AROCM were synthesized as previously reported in the literature: 23 (see Coe, J. W.; Wirtz, M. C.; Bashore, C. G.; Candler, J. Org. Lett. 2004, 6, 1589-1592) and 25 (See La, D. S.; Sattely, E. S.; Ford, J. G.; Schrock, R. R.; Hoveyda, A. H. J. Am. Chem. Soc. 2001, 123, 7767-7778) were synthesized according to the provided references.

### General Procedure for AROCM

In a glovebox, alkene 25 (40 mg, 0.2 mmol, 1 equiv) and allyl acetate (6) (140 mg, 1.4 mmol, 7 equiv) were dissolved in 0.4 mL THF. To this solution was added catalyst 4 (1.27 mg, 0.002 mmol). The reaction vial was capped and stirred for 1 h and then quenched with an excess of ethyl vinyl ether. The reaction mixture was concentrated and conversion was determined by 500 MHz ¹H NMR. The crude was subjected to flash chromatography or preparative TLC to afford the desired AROCM product (26, 33 mg, 56% yield, 15:85 Z/E ratio, 94% ee (Z), 93% ee (E)). Pure products were submitted to analytical SFC to determine ee.

### Example 36

Characterization data for AROCM product 24, 55% yield, 76:14 Z/E ratio.

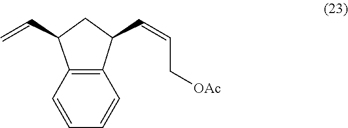 [SMILES: C=C[C@H]1C[C@@H](/C=C\COC(C)=O)c2ccccc21]

Z-24: ¹H NMR (500 MHz, CDCl₃) δ 7.25-7.20 (m, 2H), 7.19-7.14 (m, 1H), 7.11-7.07 (m, 1H), 5.89-5.81 (m, 1H), 5.80-5.75 (m, 1H), 5.67 (ddd, J=10.7, 9.6, 1.1 Hz, 1H), 5.25 (ddd, J=17.0, 1.9, 1.0 Hz, 1H), 5.18 (dd, J=10.0, 1.8 Hz, 1H), 4.78 (dt, J=6.9, 1.0 Hz, 2H), 4.15-4.03 (m, 1H), 3.76 (dt, J=10.3, 7.7 Hz, 1H), 2.54 (dt, J=12.3, 7.0 Hz, 1H), 2.11 (d, J=0.8 Hz, 2H), 1.64 (dt, J=12.2, 10.5 Hz, 1H). ¹³C NMR (125 MHz, CDCl₃) δ 145.72, 145.25, 140.55, 137.57, 127.04, 124.77, 124.30, 124.12, 116.02, 60.59, 49.13, 42.79, 41.59, 21.16. HRMS (FAB+) calculated for C₁₆H₁₇O₂ [M+H−H₂]: 241.1229. found 241.1221.

Separation conditions: AD-H, 3% IPA, 2.5 mL/min >98% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.503 | BV | 0.1472 | 6139.82520 | 668.11780 | 49.9774 |
| 2 | 3.826 | VB | 0.1547 | 6145.36768 | 625.22028 | 50.0226 |

| | | |
|---|---|---|
| Totals: | 1.22852e4 | 1293.33807 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 3.907 | BB | 0.2003 | 1.85037e4 | 1456.06311 | 100.0000 |

| | | |
|---|---|---|
| Totals: | 1.85037e4 | 1456.06311 |

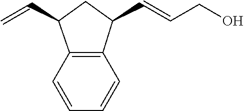

E-24 was deacetylated to the compound shown above in order to aid purification.

¹H NMR (# MHz, CDCl₃) δ 7.25-7.10 (m, 3H), 5.91-5.79 (m, 2H), 5.77-5.69 (m, 1H), 5.22 (ddd, J=17.1, 1.8, 0.9 Hz, 1H), 5.15 (dd, J=10.0, 1.9 Hz, 1H), 4.20 (t, J=5.7 Hz, 2H), 3.73 (dq, J=16.8, 8.3 Hz, 2H), 2.52 (dt, J=12.4, 7.1 Hz, 1H), 1.66 (dt, J=12.4, 10.3 Hz, 1H), 1.32 (t, J=5.7 Hz, 1H).

Separation conditions: AD-H, 3% IPA, 2.5 mL/min >98% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 12.963 | BV | 0.3805 | 1630.66382 | 52.19287 | 49.3447 |
| 2 | 13.901 | VV | 0.4567 | 1673.97461 | 49.73441 | 50.6553 |

| | | |
|---|---|---|
| Totals: | 3304.63843 | 101.92728 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 14.509 | VB | 0.5751 | 4652.46533 | 130.76849 | 100.0000 |

| | | |
|---|---|---|
| Totals: | 4652.46533 | 130.76849 |

### Example 37

Characterization data for AROCM product 26, 56% yield, 15:85 Z/E ratio.

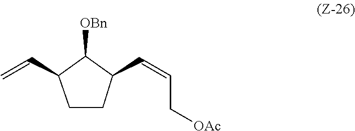 [SMILES: C=C[C@@H]1CC[C@H](/C=C\COC(C)=O)[C@@H]1OCc1ccccc1]

Z-26 [α]_{D}²⁵=−23.9° (c=0.21, CHCl₃); ¹H NMR (500 MHz, CDCl₃) δ 7.35-7.24 (m, 5H), 5.99 (ddd, J=17.1, 10.2, 8.2 Hz, 1H), 5.90-5.83 (m, 1H), 5.55 (dtd, J=11.1, 7.0, 1.0 Hz, 1H), 5.08 (ddd, J=17.2, 2.1, 1.0 Hz, 1H), 5.02 (ddd, J=10.2, 2.0, 0.8 Hz, 1H), 4.62 (dt, J=7.1, 1.1 Hz, 2H), 4.55 (d, J=11.7 Hz, 1H), 4.50 (d, J=11.7 Hz, 1H), 3.76 (t, J=4.1 Hz, 1H), 2.91 (qd, J=9.1, 4.3 Hz, 1H), 2.62 (qd, J=8.6, 3.9 Hz, 1H), 2.06 (s, 2H), 1.82 (dq, J=9.4, 6.9 Hz, 3H), 1.75-1.67 (m, 1H). ¹³C NMR (125 MHz, CDCl₃) δ 139.25, 139.09, 136.26, 128.34, 127.74, 127.52, 123.45, 115.04, 86.93, 73.76, 60.77, 50.32, 43.45, 30.53, 30.11, 28.99, 21.14. HRMS (FAB+) calculated for C₁₉H₂₄NaO₃ [M+Na]: 323.1623. found 323.1627.

Separation conditions: OJ-H, 1% IPA, 2.5 mL/min. 94% ee

Racemate:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.035 | BV | 0.2512 | 2302.19849 | 137.56712 | 50.2543 |
| 2 | 11.905 | VV | 0.2763 | 2278.89893 | 127.66735 | 49.7457 |

| | | |
|---|---|---|
| Totals: | 4581.09741 | 265.23447 |

Enantioenriched:

Signal 1: DAD1 A, Sig = 210, 8 Ref = 360,100

| Peak | RetTime | | Width | Area | Height | Area |
|---|---|---|---|---|---|---|
| # | [min] | Type | [min] | [mAU*s] | [mAU] | % |
| 1 | 11.301 | BV | 0.2214 | 239.09720 | 13.58221 | 2.6496 |
| 2 | 12.062 | VB | 0.3154 | 8784.66992 | 414.32910 | 97.3504 |

| | | |
|---|---|---|
| Totals: | 9023.76712 | 427.91131 |

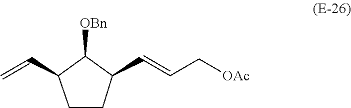 [SMILES: C=C[C@@H]1CC[C@H](/C=C/COC(C)=O)[C@@H]1OCc1ccccc1]

E-26 [α]_{D}²⁵=−1.1° (c=0.67, CHCl₃); ¹H NMR (500 MHz, CDCl₃) δ 7.40-7.23 (m, 5H), 6.07-5.97 (m, 1H), 5.95-5.88 (m, 1H), 5.61 (dt, J=15.8, 6.4 Hz, 1H), 5.09 (d, J=17.3 Hz, 1H), 5.03 (dd, J=10.4, 1.9 Hz, 1H), 4.57 (d, J=11.9 Hz, 1H), 4.54-4.51 (m, 2H), 4.49 (dd, J=11.8, 1.5 Hz, 1H), 3.79 (t, J=4.3 Hz, 1H), 2.62 (dt, J=9.7, 4.6 Hz, 2H), 2.05 (d, J=1.5 Hz, 3H), 1.87-1.75 (m, 4H). ¹³C NMR (125 MHz, CDCl₃) δ 139.37, 139.10, 136.73, 128.31, 127.82, 127.53, 124.18, 114.96, 86.98, 73.70, 65.35, 50.14, 48.54, 28.91, 21.11. HRMS (FAB+) calculated for C₁₉H₂₄NaO₃ [M+Na]: 323.1623. found 323.1628.

Separation conditions: AD-H, 2% IPA, 2.5 mL/min. 93% ee

Racemate

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU * s] | [mAU] | % |
| 1 | 5.781 | VV | 0.2561 | 3036.30420 | 188.30795 | 50.6709 |
| 2 | 6.350 | VV | 0.2732 | 2955.90186 | 174.83788 | 49.3291 |
| Totals: | | | | 5992.20605 | 363.14583 | |

Enantioenriched:

Signal 1: DAD1 A, Sig = 210, 8 Ref = 360, 100

| Peak | RetTime | | Width | Area | Height | Area |
|---|---|---|---|---|---|---|
| # | [min] | Type | [min] | [mAU * s] | [mAU] | % |
| 1 | 5.659 | VV | 0.2846 | 596.53467 | 32.73715 | 3.5544 |
| 2 | 6.433 | VV | 0.3100 | 1.61865e4 | 850.61707 | 96.4456 |
| Totals: | | | | 1.67830e4 | 883.35422 | |

The claimed invention is:

1. An enantioenriched C—H activated catalyst compound having the structure of formula (II): 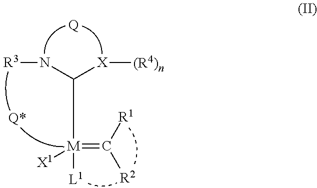
wherein,
M is a Group 8 transition metal;
L¹ is a neutral electron donor ligand;
Q* is a two electron anionic donor bridging moiety linking R³ and M;
Q is a linker selected from hydrocarbylene, substituted hydrocarbylene, heteroatom-containing hydrocarbylene, and substituted heteroatom-containing hydrocarbylene linkers;
X is an atom selected from C, N, O, S, and P;
R¹ and R² are independently selected from hydrogen, hydrocarbyl, substituted hydrocarbyl, heteroatom-containing hydrocarbyl, and substituted heteroatom-containing, hydrocarbyl, and functional groups;
R³ and R⁴ are independently selected from hydrogen, hydrocarbyl, substituted hydrocarbyl, heteroatom-containing hydrocarbyl, and substituted heteroatom-containing, hydrocarbyl, and functional groups;
n is zero, 1, or 2, such that n is zero when X is O or S, n is 1 when X is N or P, and n is 2 when X is C; and
X¹ is a bidentate anionic ligand.

2. An enantioenriched C—H activated catalyst compound, selected from

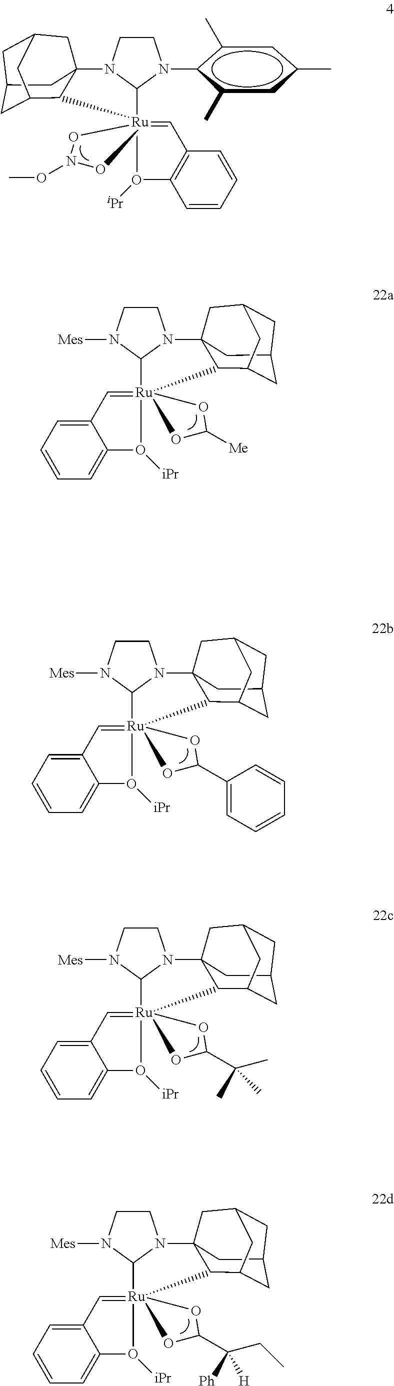

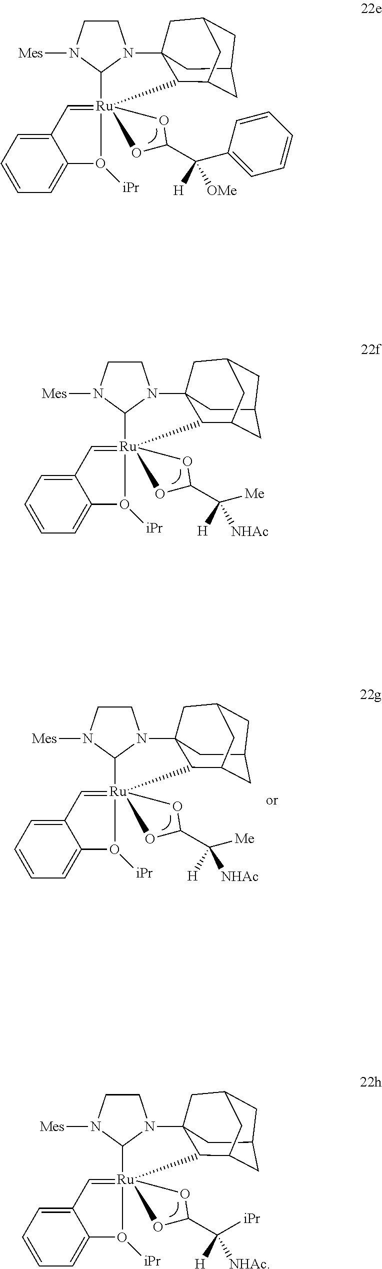

3. A method, comprising: contacting an α-olefin with a strained olefin, in the presence of an enantioenriched C—H activated catalyst under conditions and for a time period effective to allow an asymmetric ring opening cross metathesis reaction to occur.

4. The method of claim 3, wherein the strained olefin is represented by the structure of formula (XIII): 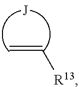
wherein R¹³ is selected from the group consisting of hydrogen, hydrocarbyl, substituted hydrocarbyl, heteroatom-containing hydrocarbyl, and substituted heteroatom-containing hydrocarbyl, wherein the substituents may be functional groups (“Fn”); and J is a saturated or unsaturated hydrocarbylene, substituted hydrocarbylene, heteroatom-containing hydrocarbylene, or substituted heteroatom-containing hydrocarbylene linkage, wherein when J is substituted hydrocarbylene or substituted heteroatom-containing hydrocarbylene, the substituents may include one or more —(Z)ₙ-Fn groups, wherein n is zero or 1.

5. The method of claim 4, wherein the strained olefin is a mono-unsaturated cyclic olefin reactant.

6. The method of claim 4, wherein the strained olefin is a bicyclic olefinic reactant or a polycyclic olefinic reactant.

7. The method of claim 3, wherein the α-olefin is represented by the structure of formula (XVIII): 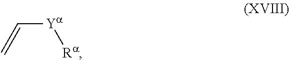
wherein,
Y^{α} is selected from the group comprising nil, CH₂, O, or S; and
R^{α} is selected from the group consisting of hydrogen, hydrocarbyl, substituted hydrocarbyl, heteroatom-containing hydrocarbyl and substituted heteroatom-containing hydrocarbyl wherein the substituents may be functional groups (“Fn”).

8. The method of claim 3, wherein the enantioenriched C—H activated catalyst has the structure of formula (II), 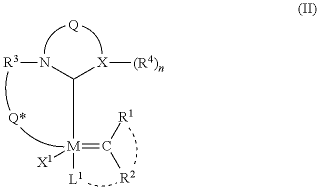
wherein,
M is a Group 8 transition metal;
L¹ is a neutral electron donor ligand;
Q* is a two electron anionic donor bridging moiety linking R³ and M;
Q is a linker selected from hydrocarbylene, substituted hydrocarbylene, heteroatom-containing hydrocarbylene, and substituted heteroatom-containing hydrocarbylene linkers;
X is an atom selected from C, N, O, S, and P;
R¹ and R² are independently selected from hydrogen, hydrocarbyl, substituted hydrocarbyl, heteroatom-containing hydrocarbyl, and substituted heteroatom-containing, hydrocarbyl, and functional groups;
R³ and R⁴ are independently selected from hydrogen, hydrocarbyl, substituted hydrocarbyl, heteroatom-containing hydrocarbyl, and substituted heteroatom-containing, hydrocarbyl, and functional groups;
n is zero, 1, or 2, such that n is zero when X is O or S, n is 1 when X is N or P, and n is 2 when X is C; and
X¹ is a bidentate anionic ligand.

9. An asymmetric ring opening cross metathesis product prepared by the method of claim 3.

10. An asymmetric ring opening cross metathesis product of claim 9 having a Z:E ratio greater than 1:1 in favor of the Z-isomer.

11. An asymmetric ring opening cross metathesis product of claim 10 having an enantiomeric excess of greater than 50%.

12. A method, comprising: reacting an α-olefin with a strained olefin, in the presence of an enantioenriched C—H activated catalyst, to form an asymmetric ring opening cross metathesis product with a Z:E ratio greater than 1:1 in favor of the Z-isomer.

13. The method of claim 12, wherein the asymmetric ring opening cross metathesis product is produced in an enantiomeric excess of greater than 50%.
